(19) United States
(12) Patent Application Publication
(10) Pub. No.: US 2020/0118164 A1
(43) Pub. Date: Apr. 16, 2020

(54) INTEGRATED MOBILE DEVICE MANAGEMENT SYSTEM

(71) Applicants: Edmond DeFrank, Northridge, CA (US); Allen Mark Jones, Imperial Beach, CA (US); Nicolas DeFrank, Porter Ranch, CA (US); Antonio DeFrank, Porter Ranch, CA (US)

(72) Inventors: Edmond DeFrank, Northridge, CA (US); Allen Mark Jones, Imperial Beach, CA (US); Nicolas DeFrank, Porter Ranch, CA (US); Antonio DeFrank, Porter Ranch, CA (US)

(21) Appl. No.: 16/532,414

(22) Filed: Aug. 5, 2019

Related U.S. Application Data
(63) Continuation-in-part of application No. 15/210,859, filed on Jul. 14, 2016, now abandoned.
Application No. 15/210,859 is a continuation-in-part of application No. 62/192,971, filed on Jul. 15, 2015.

(51) Int. Cl. G06Q 30/02; G16H 10/60; G16H 50/30; G06F 16/906; G06N 5/02

(52) U.S. Cl. CPC G06Q 30/0255; G06Q 30/0275; G06Q 30/0261; G06Q 30/0269; G16H 10/40; G16H 50/30; G06F 16/906; G06N 5/02; G16H 10/60

(57) ABSTRACT

The embodiments disclose an integrated mobile device management method including using an integrated mobile device management service provider digital programmable server and database server for coordinating processing with device issuer locked devices with functionalities specifically targeted users to limit users access to specific functions, coordinating locked devices functionalities for recording and analyzing user information, user device usage and sorting user profiles into layered categories, analyzing data and controlling function processes using at least one customized processor with an embedded algorithm within the integrated mobile device management service provider digital programmable server, downloading locked device functions to at least one locked electronic device for device issuer distribution to targeted users, using the integrated mobile device management service provider digital programmable server for operating an advertiser ad placement auction website, selecting targeted advertisements based on user device usage analysis results, and displaying targeted advertisements on at least one locked electronic devices.

### CROSS-REFERENCE TO RELATED APPLICATIONS

This continuation-in-part application is based on U.S. Provisional Patent Application Ser. No. 62/192,971 filed Jul. 15, 2015, entitled “Integrated Mobile Device Management System”, by Edmond DeFrank et al.

This continuation-in-part application is based on pending U.S. Non-Provisional application Ser. No. 15/210,859 filed Jul. 24, 2016, entitled “INTEGRATED MOBILE DEVICE MANAGEMENT SYSTEM”, by Edmond DeFrank, et al.

### BACKGROUND

The prevalence of mobile devices and varying user types compounds the difficulty advertisers in reaching a desired market group. Additionally employers who provide for example cell phones suffer from employee abuse of the availability by increasing costs through personal use rather than those intended uses for the business or service of the employer.

### BRIEF DESCRIPTION OF THE DRAWINGS

FIG. 1 shows a block diagram of an overview of integrated mobile device management system of one embodiment.

FIG. 2 shows a block diagram of an overview flow chart of integrated mobile device management system of one embodiment.

FIG. 3A shows a block diagram of an overview flow chart of input targeted user information of one embodiment.

FIG. 3B shows a block diagram of an overview flow chart of examples of layered user categories of one embodiment.

FIG. 4 shows a block diagram of an overview flow chart of locked device functionalities specifically targeted to user categories of one embodiment.

FIG. 5 shows a block diagram of an overview flow chart of limiting user access to specific functions of one embodiment.

FIG. 6 shows for illustrative purposes only an example of integrated advertisements specifically targeted to student users of one embodiment.

FIG. 7 shows for illustrative purposes only an example of integrated advertisements specifically targeted to TV station personnel users of one embodiment.

FIG. 8 shows for illustrative purposes only an example of integrated advertisements of one embodiment.

FIG. 9 shows a block diagram of a user device usage recording and analyzing functionality of one embodiment.

FIG. 10 shows a block diagram of micro-targeted market groups of users of one embodiment.

FIG. 11 shows a block diagram of an advertiser ad placement auction website of one embodiment.

FIG. 12 shows a block diagram of tracking targeted users physical locations of one embodiment.

FIG. 13 shows a block diagram of social media websites of one embodiment.

FIG. 14 shows for illustrative purposes only an example of doctor office appointment functionality of one embodiment.

FIG. 15 shows for illustrative purposes only an example of background selfie images and audio lie detection function of one embodiment.

FIG. 16 shows a block diagram of an overview flow chart of infectious disease screening function of one embodiment.

FIG. 17 shows a block diagram of an overview flow chart of electronic mental health assessment function of one embodiment.

FIG. 18 shows a block diagram of an overview flow chart of tier 2-patient questions of one embodiment.

FIG. 19 shows for illustrative purposes only an example of tier 3-patient questions of one embodiment.

FIG. 20 shows a block diagram of an overview flow chart of tier 4-patient questions of one embodiment.

FIG. 21 shows for illustrative purposes only an example of substance use and mental health in U.S. adults of one embodiment.

FIG. 22 shows for illustrative purposes only an example of locked device functionalities with hands-free devices of one embodiment.

FIG. 23 shows a block diagram of an overview flow chart of biometric devices user identification function of one embodiment.

FIG. 24 shows a block diagram of an overview flow chart of designated locked schedule and locations function of one embodiment.

FIG. 25 shows a block diagram of overview of an integrated mobile device management system home care automated auditing system functionality of one embodiment.

FIG. 26 shows for illustrative purposes only an example of a home care automated auditing system of one embodiment.

FIG. 27 shows for illustrative purposes only an example of a universal automated patient intake system of one embodiment.

FIG. 28 shows for illustrative purposes only an example of a health insurance coverage predetermination database of one embodiment.

FIG. 29 shows a block diagram of a medical condition reference materials database of one embodiment.

FIG. 30 shows for illustrative purposes only an example of a durable medical equipment reference database of one embodiment.

FIG. 31 shows for illustrative purposes only an example of a patient self-assessment testing database of one embodiment.

FIG. 32 shows for illustrative purposes only an example of a home care visiting portable electronic apparatus application of one embodiment.

FIG. 33 shows for illustrative purposes only an example of home care visit nursing auditing of one embodiment.

FIG. 34 shows for illustrative purposes only an example of home care visit DNA testing auditing of one embodiment.

FIG. 35 shows for illustrative purposes only an example of home care visit DME auditing of one embodiment.

FIG. 36 shows a block diagram of an overview of wearable health care device features of one embodiment.

FIG. 37A shows for illustrative purposes only an example of a wearable wrist device of one embodiment.

FIG. 37B shows for illustrative purposes only an example of a wearable ring device of one embodiment.

FIG. 37C shows for illustrative purposes only an example of a wearable google glasses application of one embodiment.

FIG. 37D shows for illustrative purposes only an example of a wearable glasses device of one embodiment.

FIG. 37E shows for illustrative purposes only an example of a wearable wrist band device of one embodiment.

FIG. 37F shows for illustrative purposes only an example of a wearable pendant device of one embodiment.

FIG. 38A shows for illustrative purposes only an example of a wearable glasses device features of one embodiment.

FIG. 38B shows for illustrative purposes only an example of a wearable ring device features of one embodiment.

FIG. 39A shows for illustrative purposes only an example of a wearable wrist device features of one embodiment.

FIG. 39B shows for illustrative purposes only an example of a wearable wrist device used in an examination of one embodiment.

FIG. 39C shows for illustrative purposes only an example of a wearable wrist device displaying examination results of one embodiment.

FIG. 39D shows for illustrative purposes only an example of a wearable wrist device used for writing a prescription of one embodiment.

FIG. 40 shows for illustrative purposes only an example of a tele-medicine mobile application of one embodiment.

FIG. 41 shows for illustrative purposes only an example of tele-medicine mobile application for use in remote and rural areas of one embodiment.

FIG. 42 shows for illustrative purposes only an example of a wearable glasses device used in surgery of one embodiment.

FIG. 43 shows for illustrative purposes only an example of an automated tablet and wearable device connectivity of one embodiment.

FIG. 44 shows for illustrative purposes only an example of an overview of a preventative health MACRA compliance digital system of one embodiment.

FIG. 45 shows for illustrative purposes only an example of preventive quality measures of one embodiment.

FIG. 46 shows for illustrative purposes only an example of call center and automated over the phone of one embodiment.

FIG. 47 shows for illustrative purposes only an example of automated patient portal of one embodiment.

FIG. 48 shows for illustrative purposes only an example of patient personalized prevention plan digital application of one embodiment.

FIG. 49 shows for illustrative purposes only an example of preventive care for adults patient health education of one embodiment.

FIG. 50 shows for illustrative purposes only an example of preventive care for adults patient health education (cont'd) of one embodiment.

FIG. 51 shows for illustrative purposes only an example of preventive care for women patient health education of one embodiment.

FIG. 52 shows for illustrative purposes only an example of preventive care for children patient health education of one embodiment.

FIG. 53 shows for illustrative purposes only an example of preventive care for children patient health education (cont'd) of one embodiment.

FIG. 54 shows for illustrative purposes only an example of preventive care for children patient health education (cont'd) of one embodiment.

FIG. 55 shows for illustrative purposes only an example of preventive care for pregnant women patient health education of one embodiment.

FIG. 56 shows for illustrative purposes only an example of patient health self-assessments of one embodiment.

FIG. 57 shows for illustrative purposes only an example of activities of daily living (ADL) self-assessment of one embodiment.

FIG. 58 shows for illustrative purposes only an example of chronic pain assessment questionnaire part 1 of one embodiment.

FIG. 59 shows for illustrative purposes only an example of part 1: assessment of persistent baseline pain of one embodiment.

FIG. 60 shows for illustrative purposes only an example of part 2: assessment of breakthrough pain of one embodiment.

FIG. 61 shows a block diagram of an overview of annual wellness visit of one embodiment.

FIG. 62 shows a block diagram of an overview of quality measures group 1 of one embodiment.

FIG. 63 shows a block diagram of an overview of quality measures group 2 of one embodiment.

FIG. 64 shows a block diagram of an overview of quality measures group 3 of one embodiment.

FIG. 65 shows for illustrative purposes only an example of quality measures group 4 of one embodiment.

FIG. 66 shows for illustrative purposes only an example of quality measures group 5 of one embodiment.

FIG. 67 shows for illustrative purposes only an example of quality measures group 6 of one embodiment.

FIG. 68 shows for illustrative purposes only an example of quality measures group 7 of one embodiment.

FIG. 69 shows for illustrative purposes only an example of quality measures group 8 of one embodiment.

FIG. 70 shows for illustrative purposes only an example of quality measures group 9 of one embodiment.

FIG. 71 shows for illustrative purposes only an example of quality measures group 10 of one embodiment.

FIG. 72 shows for illustrative purposes only an example of quality measures group 11 of one embodiment.

FIG. 73 shows for illustrative purposes only an example of quality measures group 12 of one embodiment.

FIG. 74 shows for illustrative purposes only an example of quality measures group 13 of one embodiment.

FIG. 75 shows for illustrative purposes only an example of benefits to health care providers of one embodiment.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized and structural changes may be made without departing from the scope of the present invention.

### GENERAL OVERVIEW

It should be noted that the descriptions that follow, for example, in terms of an integrated mobile device management system is described for illustrative purposes and the underlying system can apply to any number and multiple types of mobile devices. In one embodiment of the present invention, the integrated mobile device management system can be configured using digital processing and memory devices to operate at least one customized processor with an embedded algorithm to analyze data and control progressive examinations and testing to determine targeted conditions. The integrated mobile device management system can be configured to include functionalities specifically targeted to device issuer users and a home care automated auditing system functionality and can be configured to include integrated advertisement specifically targeted to user layered categories using the present invention.

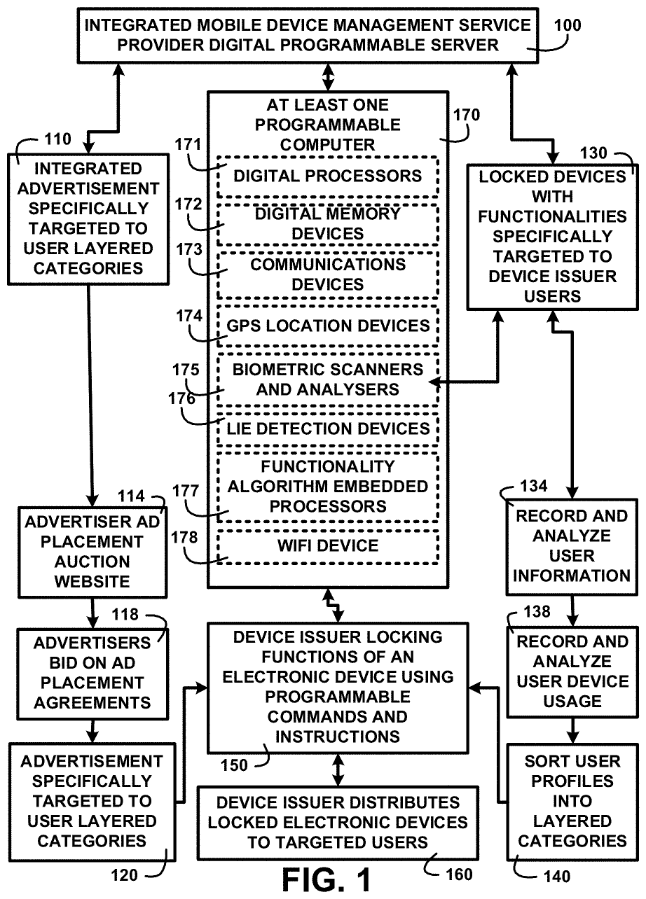

FIG. 1 shows a block diagram of an overview of integrated mobile device management system of one embodiment. FIG. 1 shows an integrated mobile device management service provider digital programmable server 100. The integrated mobile device management service provider digital programmable server 100 is used to coordinate processing with locked devices with functionalities specifically targeted to device issuer users 130. Coordinated processing with locked devices with functionalities specifically targeted to device issuer users 130 includes processes to record and analyze user information 134, to record and analyze user device usage 138 and to sort user profiles into layered categories 140. Locked devices include locked electronic devices including for example a computer, laptop, a portable electronic apparatus including a smart phone, electronic tablet, iPad, iPod, hands-free wearable health care devices including wearable wrist device, wearable ring device, Google glasses, wearable glasses device, wearable wrist band device, wearable pendant device and other digitally programmable devices. Uses of device issuer locked electronic devices are controlled using device issuer locking functions of an electronic device using programmable commands and instructions 150. A device issuer distributes locked electronic devices to targeted users 160.

The integrated mobile device management service provider digital programmable server 100 and locked devices are configured to include at least one coordinated processing function including a function to display at least one integrated advertisement specifically targeted to classify user layered categories 110. The integrated mobile device management service provider digital programmable server 100 includes an advertiser ad placement auction website 114. The advertiser ad placement auction website 114 is used to promote advertisement specifically targeted to user layered categories 120 wherein advertisers bid on ad placement agreements 118.

The integrated mobile device management service provider digital programmable server 100 includes at least one programmable computer 170. The at least one programmable computer 170 is configured to include digital processors 171, digital memory devices 172, communications devices 173, GPS location devices 174, biometric scanners and analyzers 175, lie detection devices 176, functionality algorithm embedded processors 177 and at least one WIFI device 178 to process data and perform calculations to make analytical determinations of expected and unexpected analysis results. At least one functionality algorithm embedded processor is used to process and calculate locked device functionalities.

Locked devices with functionalities specifically targeted to device issuer users 130 include device issuer locking functions of an electronic device using programmable commands and instructions 150. Device issuer locking functions are configured for device issuer tasks and operations which are downloaded into locked electronic devices prior to the time when a device issuer distributes locked electronic devices to targeted users 160 of one embodiment.

### DETAILED DESCRIPTION

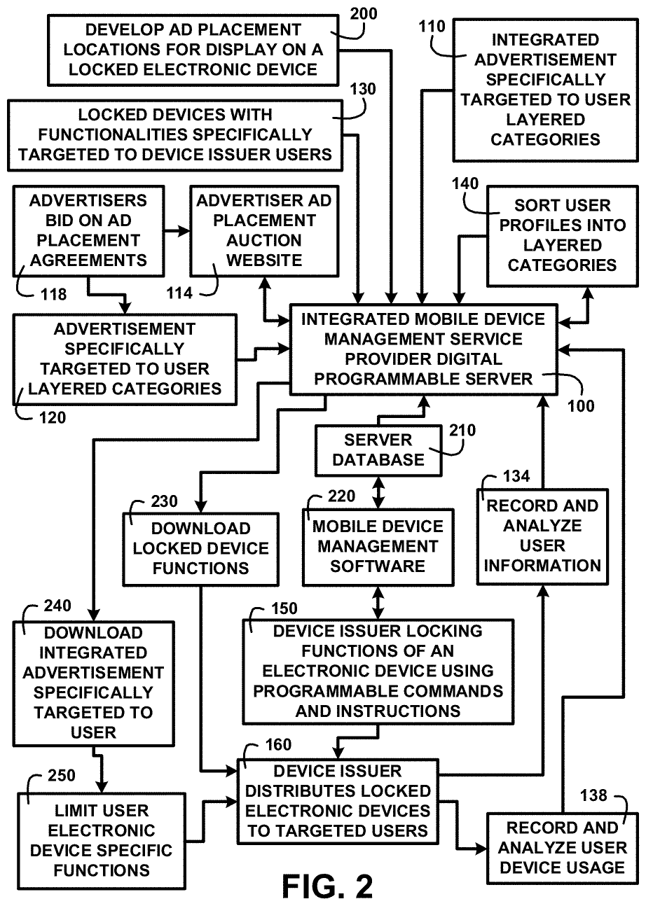

FIG. 2 shows a block diagram of an overview flow chart of integrated mobile device management system of one embodiment. FIG. 2 shows the integrated mobile device management service provider digital programmable server 100 configured to develop ad placement locations for display on a locked electronic device 200 and to develop locked device functionalities specifically targeted to device issuer users 130. Ad placement locations are used where advertisers bid on ad placement agreements 118 on the advertiser ad placement auction website 114. The advertisement specifically targeted to user layered categories 120 includes integrated advertisement specifically targeted to user layered categories 110 developed wherein a server database 210 stored data is used to sort user profiles into layered categories 140.

The server database 210 includes mobile device management software 220 including device issuer locking functions of an electronic device using programmable commands and instructions 150. The server database 210 is used to download locked device functions 230 and to download integrated advertisement specifically targeted to user 240 into the locked electronic devices. The device issuer distributes locked electronic devices to targeted users 160 wherein the downloaded locked device functions limit user electronic device specific functions 250, record and analyze user information 134 and record and analyze user device usage 138 of one embodiment.

Input Targeted User Information:

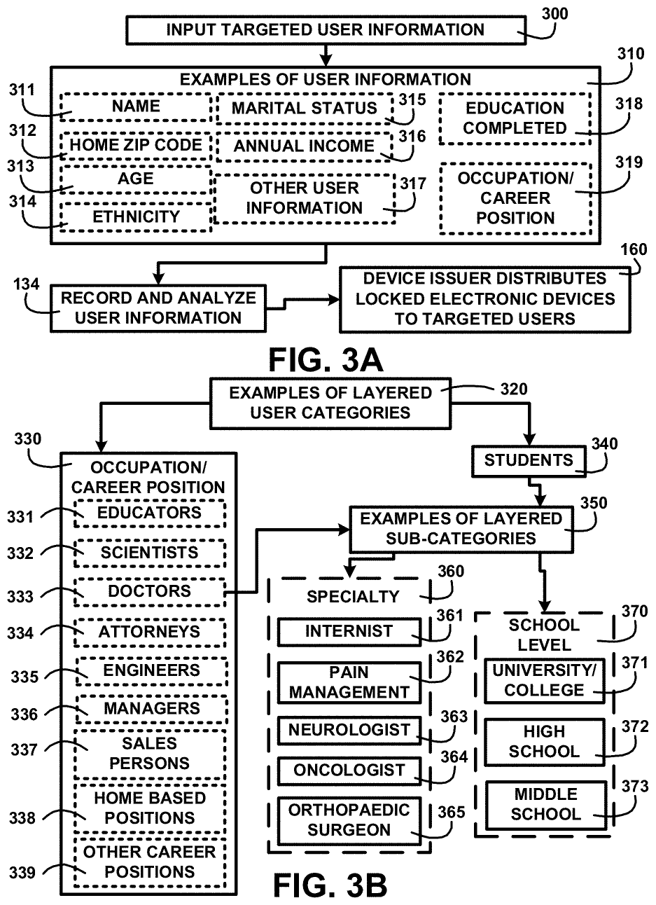

FIG. 3A shows a block diagram of an overview flow chart of input targeted user information of one embodiment. FIG. 3A shows the device issuer can input targeted user information 300 into a locked device prior to distribution to a targeted user. Examples of user information 310 include name 311, home zip code 312, age 313, ethnicity 314, marital status 315, annual income 316, other user information 317, education completed 318, and occupation/career position 319. The server database 210 is used to record and analyze user information 134 inputted into the locked device. The device issuer distributes locked electronic devices to targeted users 160 of one embodiment.

Examples of Layered User Categories:

FIG. 3B shows a block diagram of an overview flow chart of examples of layered user categories of one embodiment. FIG. 3B shows examples of layered user categories 320 including examples of layered sub-categories 350. Layered user categories include for example an occupation/career position 319 including educators 331, scientists 332, doctors 333, attorneys 334, engineers 335, managers 336, sales persons 337, home based positions 338 and other career positions 339. Layered sub-categories of the occupation/career position 319 layered user category includes for example doctors 333, specialty 360, internist 361, pain management 362, neurologist 363, oncologist 364, and orthopedic surgeon 365. Another example of layered user categories is students 340. Layered sub-categories of the students 340 layered user category include for example school level 370 including for example university/college 371, high school 372, and middle school 373 of one embodiment.

Locked Device Functionalities Specifically Targeted to User Categories:

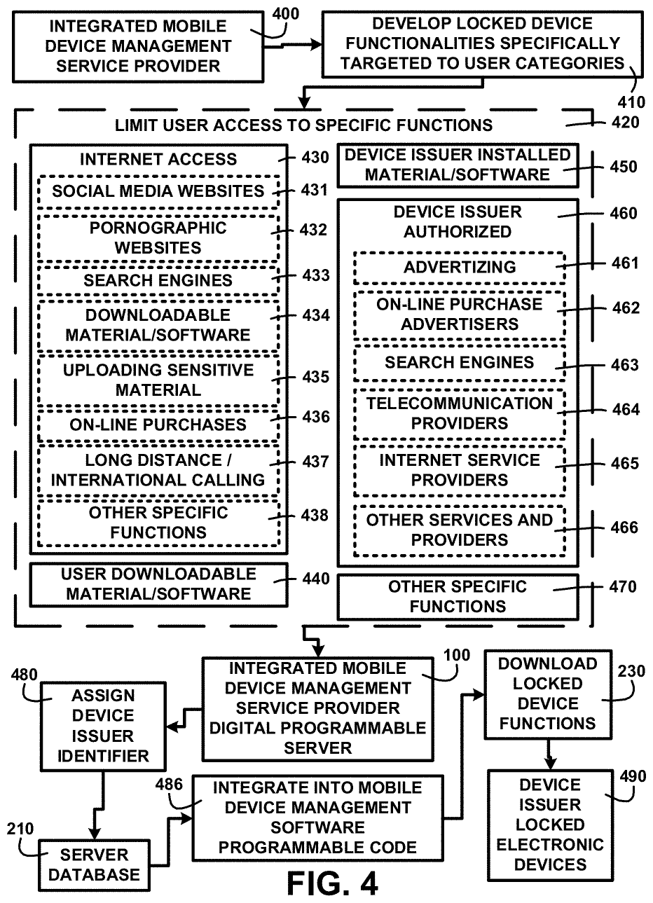

FIG. 4 shows a block diagram of an overview flow chart of locked device functionalities specifically targeted to user categories of one embodiment. FIG. 4 shows an integrated mobile device management service provider 400 used to develop locked device functionalities specifically targeted to user categories 410. The integrated mobile device management service provider 400 is used to limit user access to specific functions 420 including Internet access 430 including social media websites 431, pornographic websites 432, search engines 433, downloadable material/software 434, uploading sensitive material 435, on-line purchases 436, long distance/international calling 437, and other specific functions 438.

The integrated mobile device management service provider 400 is used to limit user access to specific functions 420 including user downloadable material/software 440, device issuer installed material/software 450, and device issuer authorized 460 advertising 461, on-line purchase advertisers 462, search engines 433, telecommunication providers 464, Internet service providers 465, other services and providers 466, and other specific functions 438.

The integrated mobile device management service provider digital programmable server 100 is used to assign device issuer identifier 480 which is recorded in the server database 210 and is processed to integrate into mobile device management software programmable code 486 into the download locked device functions 230 on device issuer locked electronic devices 490. The device issuer locked electronic devices 490 include computers, laptop computers, tablets, smart phones, iPods, electronic tablets, iPads, and other digital electronic devices.

For example a device issuer locked electronic device can be configured to include a locked laptop computer for use by an employee in a home based position. The locked laptop computer used in the employee home can be configured to include at least one employee work hour monitoring functionality, wherein during periods of time the employee is performing work duties on the locked laptop computer hours of work are being determine using an algorithm to record and calculate increments of time that include a login, key strokes, queries to a company server, uploaded completed work product, intra-company communications and communications for example to customers including telephonic, fax and email, and other actions and forms of data that are directly associated with the home based position. The device issuer for example an employer of the home based employee using the determination of work hours can evaluate and analyze for example actual time spent working and productivity.

The locked laptop computer in this example can be configured to include biometric device features and functions to for example determine a user identification and truthfulness in answering questions. The device issuer locked electronic devices 490 are configured to include at least one functionality algorithm embedded processor of one embodiment.

Limiting User Access to Specific Functions:

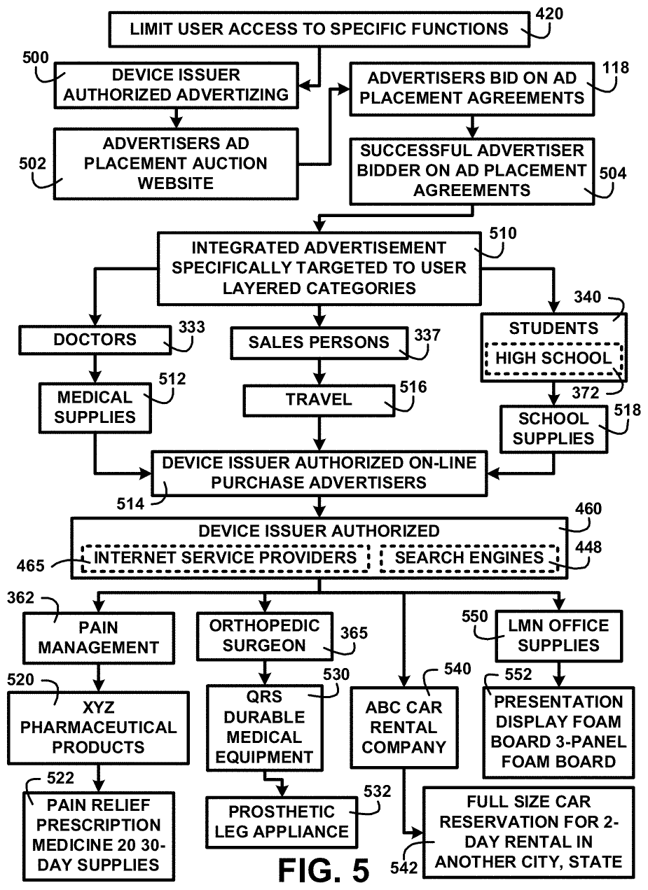

FIG. 5 shows a block diagram of an overview flow chart of limiting user access to specific functions of one embodiment. FIG. 5 shows the process to limit user access to specific functions 420 includes device issuer authorized advertising 500. The device issuer authorized advertising 500 is loaded from a advertiser ad placement auction website 502 based on advertiser bid on ad placement agreements 118 for a successful advertiser bidder on ad placement agreements 504. The device issuer authorized advertising 500 includes integrated advertisement specifically targeted to user layered categories 510 including for example doctors 333 seeking medical supplies 512 using device issuer authorized on-line purchase advertisers 514. The device issuer authorized advertising 500 includes integrated advertisement specifically targeted to user layered categories 510 including for example sales persons 337 planning travel 516 using device issuer authorized on-line purchase advertisers 514. The device issuer authorized advertising 500 includes integrated advertisement specifically targeted to user layered categories 510 including for example students 340 in high school 372 desiring school supplies 518 using device issuer authorized on-line purchase advertisers 514.

Another process to limit user access to specific functions 420 includes device issuer authorized 460 Internet service providers 465 and search engines 433. For example a search for pain management 362 displays XYZ pharmaceutical products 520 including pain relief prescription medicine 20 30-day supplies 522. Another example a search by an orthopedic surgeon 365 for QRS durable medical equipment 530 displays a prosthetic leg appliance 532 from device issuer authorized on-line purchase advertisers 514. For example a travel 516 search may display ABC car rental company 540 one of device issuer authorized on-line purchase advertisers 514 for a full size car reservation for 2-day rental in another city, state 542. Other examples can include a search for office supplies and the result is displayed from LMN office supplies 550 for presentation display foam board 3-panel foam board 552 one of device issuer authorized on-line purchase advertisers 514 of one embodiment.

Integrated Advertisements Specifically Targeted to Student Users:

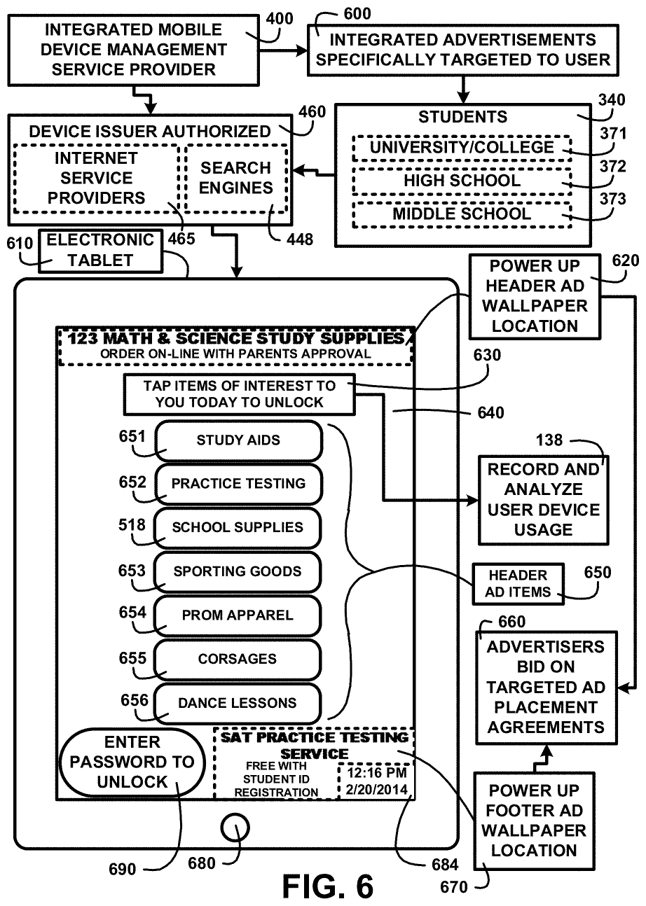

FIG. 6 shows for illustrative purposes only an example of integrated advertisements specifically targeted to student users of one embodiment. FIG. 6 shows the integrated mobile device management service provider 400 providing the device issuer authorized 460 Internet service providers 465 and search engines 433. The integrated mobile device management service provider 400 directs integrated advertisements specifically targeted to user 600. The integrated advertisements specifically targeted to user 600 include for example students 340 attending university/college 371, high school 372 or middle school 373. The students 340 may be using an electronic tablet 610 provided by the device issuer.

The electronic tablet 610 provided by the device issuer can for example display a power up header ad wallpaper location 620 showing 123 math & science study supplies order on-line with parent's approval. The user may use the device 630 to tap items of interest to you today to unlock. Items of interest may include for example items on a power up screen wallpaper display 640 from the header ad items 650 including for example study aids 651, practice testing 652, school supplies 518, sporting goods 653, prom apparel 654, corsages 655, and dance lessons 656. The electronic tablet 610 will record and analyze user device usage 138.

Advertisers bid on targeted ad placement agreements 660 for ad placement including a power up footer ad wallpaper location 670 for displaying targeted advertisements including for example SAT practice testing service free with student ID registration. The electronic tablet 610 includes a tablet on/off button 680, date and time display 684 for example 12:15:00 pm 2/20/2014 showing a current date and time display 685 and a touch screen area with an unlock instruction for example enter password to unlock 690 of one embodiment.

Integrated Advertisements Specifically Targeted to TV Station Personnel Users:

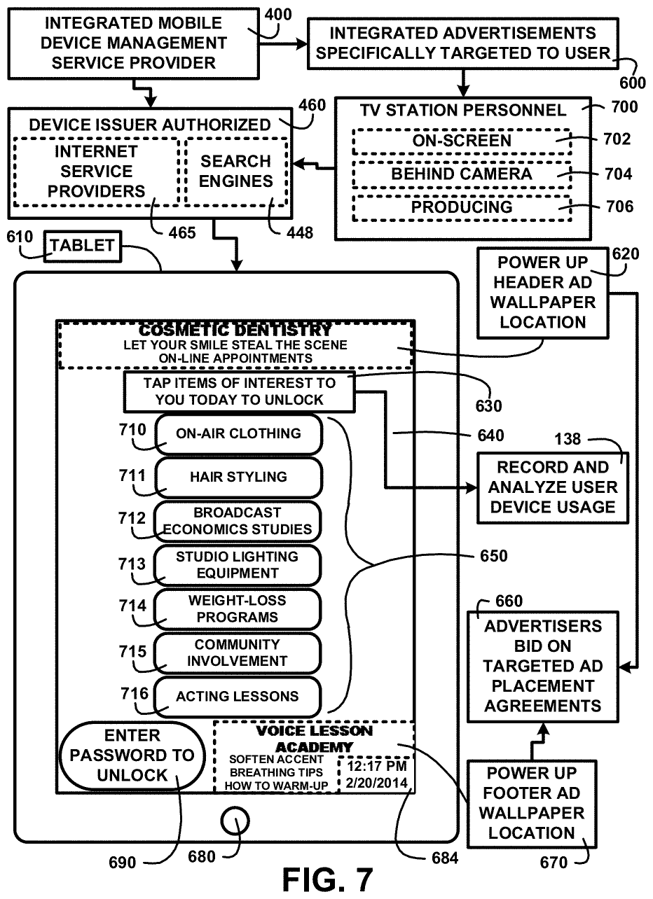

FIG. 7 shows for illustrative purposes only an example of integrated advertisements specifically targeted to TV station personnel users of one embodiment. FIG. 7 shows the integrated mobile device management service provider 400 providing the device issuer authorized 460 Internet service providers 465 and search engines 433. The integrated mobile device management service provider 400 directs integrated advertisements specifically targeted to user 600. The user targeted may be for example TV station personnel 700 including for example on-screen 702 persons, behind camera 704 and producing 706 personnel. The power up header ad wallpaper location 620 may include for example Cosmetic Dentistry; Let your smile steal the scene, on-line appointments.

The power up screen wallpaper display 640 allows the user to use the device 630 to tap items of interest to you today to unlock where the items of interest are downloaded according to advertisers bid on targeted ad placement agreements 660. The items of interest in this example may include for example on-air clothing 710, hair styling 711, broadcast economics studies 712, studio lighting equipment 713, weight-loss programs 714, community involvement 715, and acting lessons 716. The electronic tablet 610 is configured to record and analyze user device usage 138. Also displayed is the power up footer ad wallpaper location 670 presenting for example a voice lesson academy offering services to soften accent, breathing tips, how to warm-up.

The electronic tablet 610 includes a tablet on/off button 680, date and time display 684 for example 12:15:00 pm 2/20/2014 showing a current date and time display 685 and a touch screen area with an unlock instruction for example enter password to unlock 690 of one embodiment.

Integrated Advertisements:

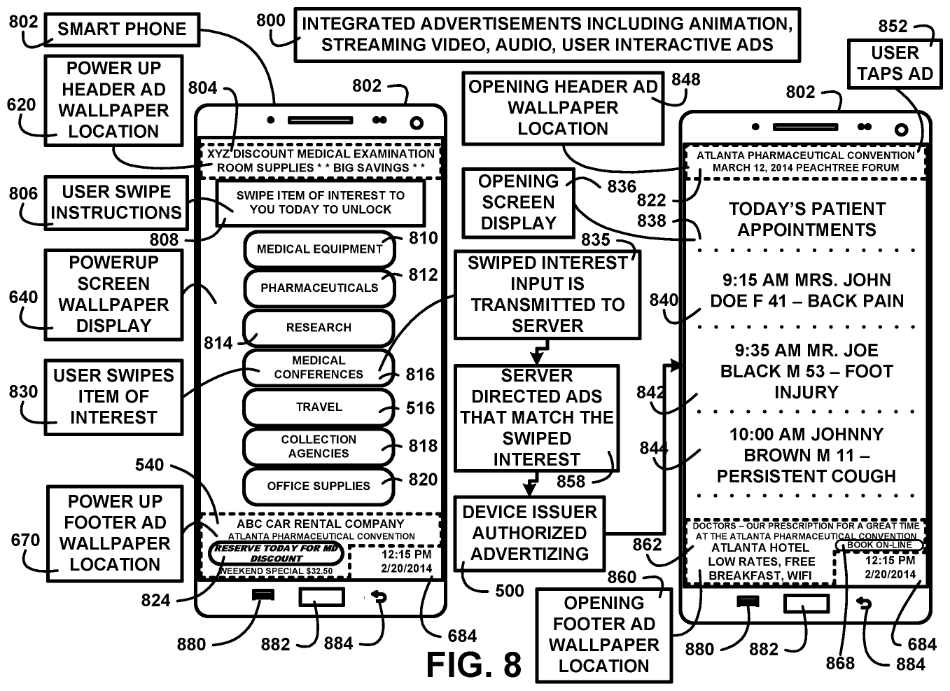

FIG. 8 shows for illustrative purposes only an example of integrated advertisements of one embodiment. FIG. 8 shows integrated advertisements including animation, streaming video, audio, user interactive ads 800. Integrated advertisements are shown on a smart phone 802 embodiment of device issuer locked electronic devices 490. The smart phone 802 includes a menu touch point 880, power-on touch point 882, a back button touch point 884 and date and time display 684 for example 12:15:00 pm 2/20/2014. The power up header ad wallpaper location 620 displays for example xyz discount medical examination room supplies * * big savings * * 804 where * * big savings * * can be configured for example to blink on and off, change colors and the star elements appear to be sparkling. Also displayed are user swipe instructions 806 for example swipe item of interest to you today to unlock 808.

The power up screen wallpaper display 640 includes medical equipment 810, pharmaceuticals 812, research 814, medical conferences 816, travel 516, collection agencies 818, and office supplies 820. An example of user interactive ads includes for example a process where prior to swiping an item of interest the user can tap an item of interest which will then display a streaming video of images of items of interest for example medical equipment 810. The user in sees an image of an item of interest the user may be reminded that they desire that specific item.

Another example of user interactive ads includes for example a process where prior to swiping an item of interest the user can tap an item of interest which will then audibly announce for example medical conferences 816 which correspond to the user category for example medical conferences 816 on orthopedics. When the user taps the medical conferences the power up footer ad wallpaper location 670 is displayed showing when the orthopedic medical conference in Atlanta is audibly announced. The power up footer ad wallpaper location 670 shows ABC car rental company 540, Atlanta Pharmaceutical Convention 822, reserve today for MD discount 824, and weekend special $32.50 826.

When a user swipes item of interest 830 the swiped interest input is transmitted to server 835. The user smart phone 802 is unlocked and displays Atlanta Pharmaceutical Convention Mar. 12, 2014 Peachtree Forum 822 reflecting the item of interest swiped by the user in an opening header ad wallpaper location 848. An opening screen display 836 is used to display today's patient appointments 838 including 9:15 am Mrs. John Doe f 41-back pain 840, 9:35 am Mr. Joe Black m 53-foot injury 842, and 10:00 am Johnny Brown m 11-persistent cough 844. This display has been pre-programmed by the device issuer to remind the user of the day's schedule.

Server directed ads that match the swiped interest 858 and include device issuer authorized advertising 500 is displayed in an opening footer ad wallpaper location 860 including for example doctors—our prescription for a great time at the Atlanta pharmaceutical convention 862 Atlanta hotel low rates, free breakfast, Wi-Fi 864 and a book on-line 868 selection touch screen area. When the user makes the decision to learn more about the medical convention the user taps ad 852 of one embodiment.

A User Device Usage Recording and Analyzing Functionality:

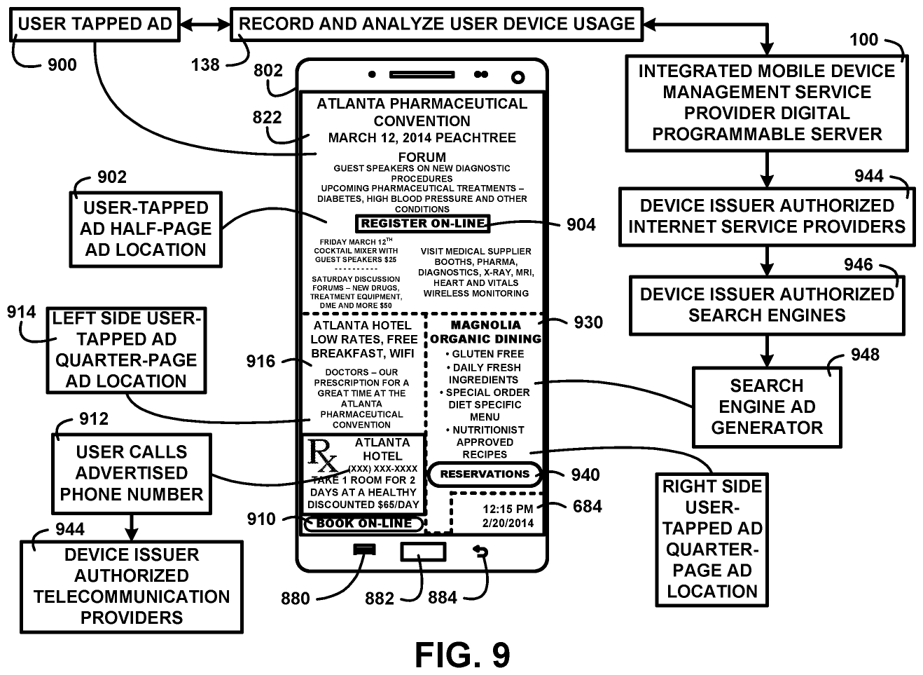

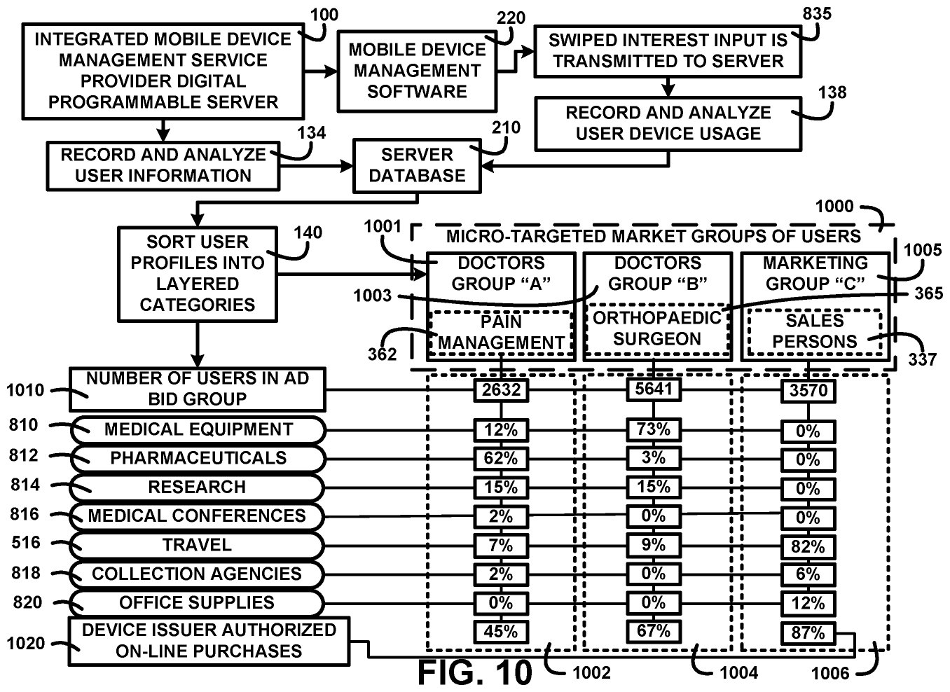

FIG. 9 shows a block diagram of a user device usage recording and analyzing functionality of one embodiment. FIG. 9 shows the user smart phone 802 displaying a user tapped ad 900 for example the Atlanta Pharmaceutical Convention Mar. 12, 2014 Peachtree Forum 822. The user smart phone 802 includes the menu touch point 880, power-on touch point 882, back button touch point 884 and date and time display 684 for example 12:15:00 pm 2/20/2014 for a current date and time display 685. A user-tapped ad half-page ad location 902 is used to display details of the exampled medical conference including guest speakers on new diagnostic procedures, and upcoming pharmaceutical treatments—diabetes, high blood pressure and other conditions. The half-page ad also displays for example Friday March 12th cocktail mixer with guest speakers $25, Saturday discussion forums—new drugs, treatment equipment, DME and more $50, and visit medical supplier booths, pharma, diagnostics, X-ray, MRI, heart and vitals wireless monitoring. The exampled ad further includes a register on-line 904 touch screen area.

A left side user-tapped ad quarter-page ad location 914 displays for example Atlanta Hotel low rates, free breakfast, WI-FI 916, doctors—our prescription for a great time at the Atlanta Pharmaceutical Convention, Rx Atlanta Hotel, instructions for user calls advertised phone number 912 for example (xxx) xxx-xxxx, take 1 room for 2 days at a healthy discounted $65/day, and a book on-line touch screen button 910.

A right side user-tapped ad quarter-page ad location 928 displays for example Magnolia Organic Dining 930, gluten free, daily fresh ingredients, special order diet specific menu, nutritionist approved recipes, and a reservations touch screen button 940. The user tapped ad 900 automatically activates a process to record and analyze user device usage 138 on the integrated mobile device management service provider digital programmable server 100. The integrated mobile device management service provider digital programmable server 100 directs to device issuer authorized Internet service providers 944 and device issuer authorized search engines 946 device issuer authorized integrated advertisements from a search engine ad generator 948 of one embodiment.

Micro-Targeted Market Groups of Users:

FIG. 10 shows a block diagram of micro-targeted market groups of users of one embodiment. FIG. 10 shows the integrated mobile device management service provider digital programmable server 100 is used to record and analyze user information 134 on the server database 210. The mobile device management software 220 is used in a process where swiped interest input is transmitted to server 835. The swiped interest input reflects a user device usage that is processed to record and analyze user device usage 138 and stored in the server database 210.

The integrated mobile device management service provider digital programmable server 100 is used to sort user profiles into layered categories 140. FIG. 10 shows an example of micro-targeted market groups of users 1000 including for example doctors group “A” 1001, doctors group “B” 1003, and marketing group “C” 1005. Layered sub-categories 350 of FIG. 3 include for example pain management 362, orthopedic surgeon 365, and sales persons 337 associated respectively with the micro-targeted market groups of users 1000 examples.

The analysis processed using the integrated mobile device management service provider digital programmable server 100 provides for example a doctors group “A” usage percentage 1002, a doctors group “B” usage percentage 1004, and a marketing group “C” usage percentage 1006 for a number of users in ad bid group 1010. The usage percentages for each of the layered sub-categories 350 of FIG. 3 are shown for various items of interest including for example medical equipment 810, pharmaceuticals 812, research 814, medical conferences 816, travel 516, collection agencies 818, office supplies 820, including device issuer authorized on-line purchases 1020 of one embodiment.

An Advertiser Ad Placement Auction Website:

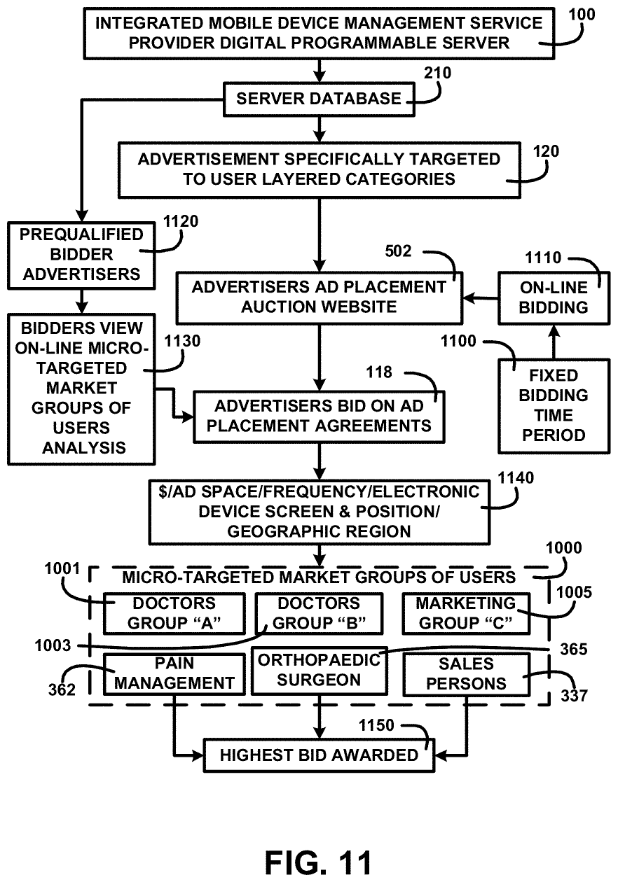

FIG. 11 shows a block diagram of an advertiser ad placement auction website of one embodiment. FIG. 11 shows the integrated mobile device management service provider digital programmable server 100 using server database 210 user device usage to develop advertisement specifically targeted to user layered categories 120. The advertisement specifically targeted to user layered categories are used on the advertiser ad placement auction website 502 to display the usage percentages for each of the user layered categories and layered sub-categories 350 of FIG. 3. An auction is held for a fixed bidding time period 1100 for on-line bidding 1110 by prequalified bidder advertisers 1120. Bidders view on-line micro-targeted market groups of user analysis 1130. Advertisers bid on ad placement agreements 118 for $/ad space/frequency/electronic device screen & position/geographic region 1140 to display ads to micro-targeted market groups of users 1000.

The micro-targeted market groups of users 1000 includes for example doctors group “A” 1001 pain management 362, doctors group “B” 1003 orthopedic surgeon 365, and marketing group “C” 1005 sales persons 337. A highest bid awarded 1150 is made in an agreement for each item of interest, for each micro-targeted market groups of users 1000 and $/ad space/frequency/electronic device screen & position/geographic region 1140 factors of one embodiment.

Tracking Targeted User's Physical Locations:

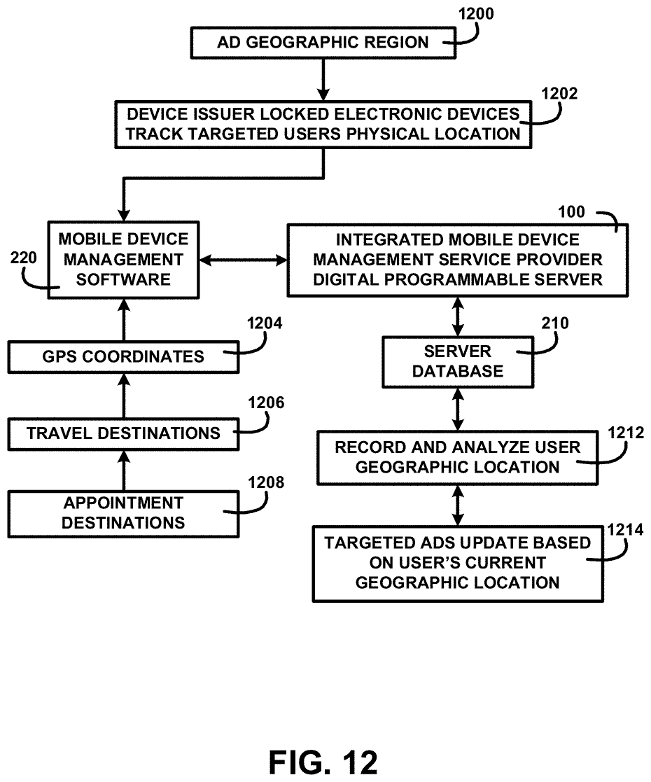

FIG. 12 shows a block diagram of tracking targeted users physical locations of one embodiment. FIG. 12 shows ad geographic region 1200 determinations using device issuer locked electronic devices track targeted users physical location 1202. The device issuer locked electronic devices track targeted users physical location 1202 is recorded and transmitted to the integrated mobile device management service provider digital programmable server 100 using the mobile device management software 220 GPS coordinates 1204.

The mobile device management software 220 using the GPS coordinates 1204 captures travel destinations 1206 and appointment destinations 1208. The captured travel destinations 1206 and appointment destinations 1208 GPS coordinates 1204 are transmitted to the server database 210 and recorded. The captured travel destinations 1206 and appointment destinations 1208 GPS coordinates 1204 recorded in the server database 210 are used by the integrated mobile device management service provider digital programmable server 100 to record and analyze user geographic location 1212 for each destination.

The analysis of the captured GPS coordinates 1204 is used to determine targeted ads update based on user's current geographic location 1214. While the user is in a current geographic location the targeted ads are selected from advertisers within a predetermined radius of the user current geographic location. The predetermination of the radius is made using the user selection of items of interest, for example if the user selected a hotel room and rental car at the current location geographic location it would indicate the user is traveling. The distance of the radius is reduced as it may be problematic for the user to drive a greater distance in a potentially unfamiliar region away from home.

In an alternative if the server database 210 data shows the user making frequent trips to the same travel destination the historical user geographic locations can be used to determine how extensively the user moved within the travel destination region and for example to farthest distance traveled from for example a user hotel can be used to determine the radius of targeted ads update based on user's current geographic location 1214 of one embodiment.

Social Media Websites:

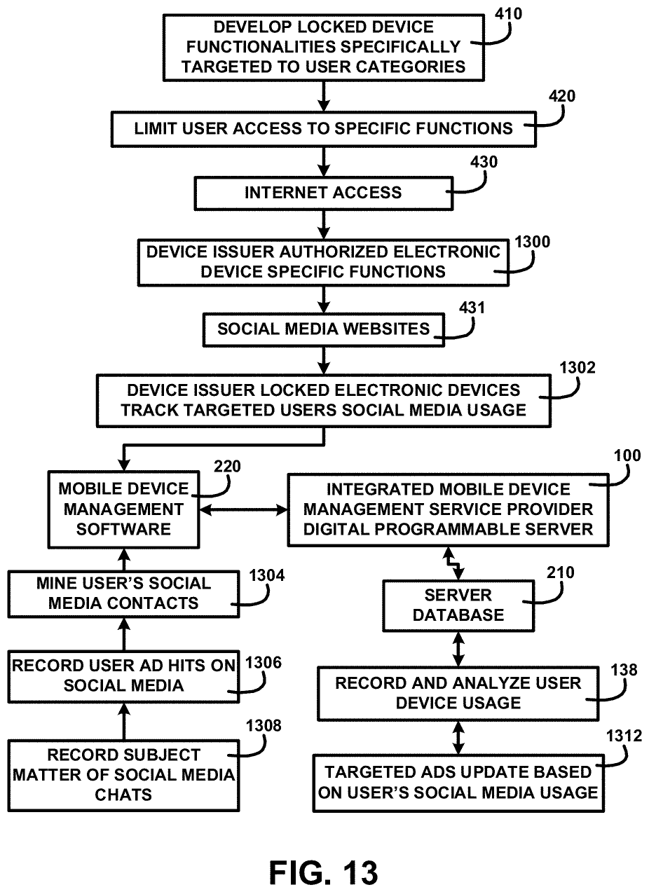

FIG. 13 shows a block diagram of social media websites of one embodiment. FIG. 13 shows a process to develop locked device functionalities specifically targeted to user categories 410 to limit user access to specific functions 420. Limited user access to specific functions includes for example Internet access 430; device issuer authorized electronic device specific functions 1300, and social media websites 431. Device issuer locked electronic devices track targeted users social media usage 1302 using the mobile device management software 220.

The mobile device management software 220 can be used to for example mine user's social media contacts 1304, record user ad hits on social media 1306, and record subject matter of social media chats 1308. The integrated mobile device management service provider digital programmable server 100 and server database 210 can be used to record and analyze user device usage 138 and direct targeted ads update based on user's social media usage 1312 of one embodiment.

Doctor Office Appointment Functionality:

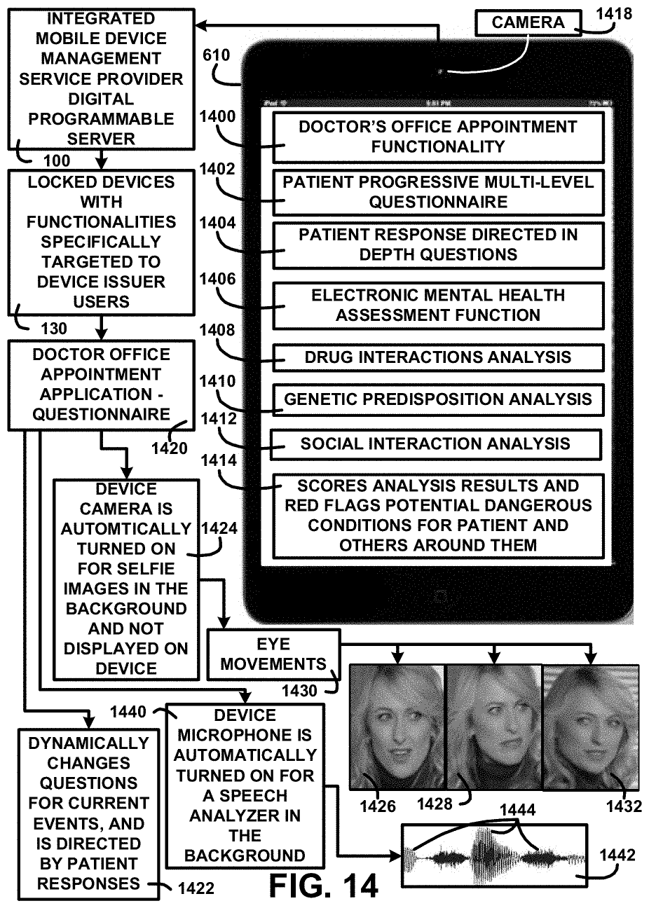

FIG. 14 shows for illustrative purposes only an example of doctor office appointment functionality of one embodiment. FIG. 14 shows the electronic tablet 610 with a doctor's office appointment functionality 1400 downloaded from the integrated mobile device management service provider digital programmable server 100. The doctor office appointment functionality 1400 includes for example a patient progressive multi-level questionnaire 1402, patient response directed in depth questions 1404, electronic mental health assessment function 1406, drug interactions analysis 1408, genetic predisposition analysis 1410, social interaction analysis 1412, and scores analysis results and red flags potential dangerous conditions for patient and others around them 1414.

The electronic tablet 610 can be configured for a coordinated care digital electronic platform in a doctor's office and a health provider's office. The electronic tablet 610 can be configured to develop locked device functionalities specifically targeted to user categories 410 of FIG. 4 to limit user access to specific functions 420 of FIG. 4. For example the tablet coordinated care digital electronic platform is configured “locked” in a kiosk mode so only applications from the integrated mobile device management service provider digital programmable server are accessible. These features ensure patient confidentially under HIPAA laws and compliance with Stark laws and federal and state Anti-Kickback laws. The tablet coordinated care digital electronic platform may qualify as a Class II Mobile Medical App Platform.

The tablet coordinated care digital electronic platform is a personalized healthcare platform for physicians. The tablet coordinated care digital electronic platform includes a suite of applications that are designed to take advantage of new Affordable Care Act (ACA) provisions, aid patient care, and maximizes physician revenue capture for medically necessary ACA tests. The tablet coordinated care digital electronic platform includes tools for automating tasks and aggregating, manipulating and integrating data into daily workflows. The tablet coordinated care digital electronic platform allows information to move seamlessly between the tablet, the exam room, and the health records system.

The tablet coordinated care digital electronic platform can provide electronic integration and electronic connectivity with for example a doctor's office management system. The integrated mobile device management service provider can integrate with a doctor's office management system using for example HL7 LIMS and LIS Integration. The integrated mobile device management service provider can integrate the tablet coordinated care digital electronic platform by electronically connecting the tablet coordinated care digital electronic platform to the integrated mobile device management service provider's LIMS and/or LIS, as necessary, to perform all of said electronic services offered by the integrated mobile device management service provider.

The tablet coordinated care digital electronic platform includes a suite of applications including for example Interactive Mental Health Assessments/Wellness Questionnaires, Neurocognitive testing, Annual Wellness Visits, Chronic Care Management, Allergy Testing and Immunotherapy Program, DNA Testing including Cancer DNA testing and DNA FDA Cleared Cystic Fibrosis testing and Toxicology testing. The tablet coordinated care digital electronic platform will include a physician “app store” that will allow physicians to access third party apps. A third party app store will allow a physician to pick and choose apps suited for that physician.

The integrated mobile device management service provider digital programmable server 100 has been used to develop locked device functionalities specifically targeted to device issuer users 130 including a doctor office appointment application-questionnaire 1420 including for example the patient progressive multi-level questionnaire 1402. The patient progressive multi-level questionnaire 1402 dynamically changes questions for current events, and is directed by patient responses 1422. The electronic tablet 610 device includes a camera 1418 wherein a device camera is automatically turned on for selfie images in the background and not displayed on device 1424. The selfie images captured by the camera 1418 can record patient eye movements 1430 including changing eye movements 1432, normal expressions 1426 and concerned expressions 1428. A device microphone is automatically turned on for a speech analyzer in the background 1440. The speech analyzer is used to graph of speech patterns 1442. The speech analyzer used the graph of speech patterns 1442 to determine and analyze rising, slowing, changes in pitch, loudness, and quality 1444 of the speech patterns of one embodiment.

Background Selfie Images and Audio Lie Detection Function:

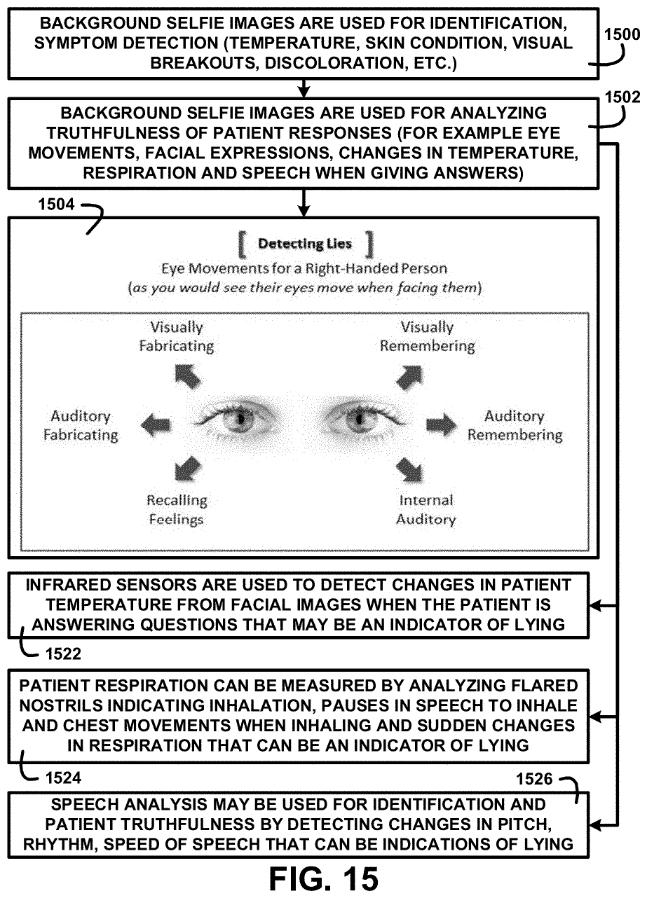

FIG. 15 shows for illustrative purposes only an example of background selfie images and audio lie detection function of one embodiment. FIG. 15 shows at least one function used to detect whether a patient is being truthful when answering for example the patient progressive multi-level questionnaire 1402. Background selfie images are used for identification, symptom detection (temperature, skin condition, visual breakouts, discoloration, etc.) 1500.

Background selfie images are used for analyzing truthfulness of patient responses (for example eye movements, facial expressions, changes in temperature, respiration and speech when giving answers) 1502. For example industry standards for detecting lies 1504 include using eye movements 1430 of FIG. 14 including changing eye movements 1432 of FIG. 14, normal expressions 1426 of FIG. 14 and concerned expressions 1428 of FIG. 14, body temperature changes, respiration and other physiological responses. The physiological responses captured using the electronic tablet 610 with a doctor's office appointment functionality 1400 downloaded can be analyzed using for example the selfie images, for example eye movements for a right-handed person (as you would see their eyes move when facing them), visually fabricating, auditory fabricating, recalling feelings, visually remembering, auditory remembering, and internal auditory.

Infrared sensors are used to detect changes in patient temperature from facial images when the patient is answering questions that may be an indicator of lying 1522. Patient respiration can be measured by analyzing flared nostrils indicating inhalation, pauses in speech to inhale and chest movements when inhaling and sudden changes in respiration that can be an indicator of lying 1524 captured while the patient is answering a questionnaire. A speech analysis may be used for identification and patient truthfulness by detecting changes in pitch, rhythm, speed of speech that can be indications of lying 1526. Sounds including speech may be generally characterized by pitch, loudness, and quality. Sound “quality” or “timbre” describes those characteristics of sound which allow the ear to distinguish sounds which have the same pitch and loudness.

The changes in sound values analyzed from the data used to depict the graph of speech patterns 1442 of FIG. 14 may be undiscernible to a listener but can be detectable to the device microphone and registered by the speech analyzer. The speech analyzer using at least one customized processor with an embedded algorithm to analyze sound can detect the changes in the speech pattern and send analytical results to for example the doctor's office staff data to provide an assessment of the patient truthfulness in answering the questionnaire. The results may show for example that a patient is experiencing pain even though they answered no to a question asking that question of one embodiment.

Infectious Disease Screening:

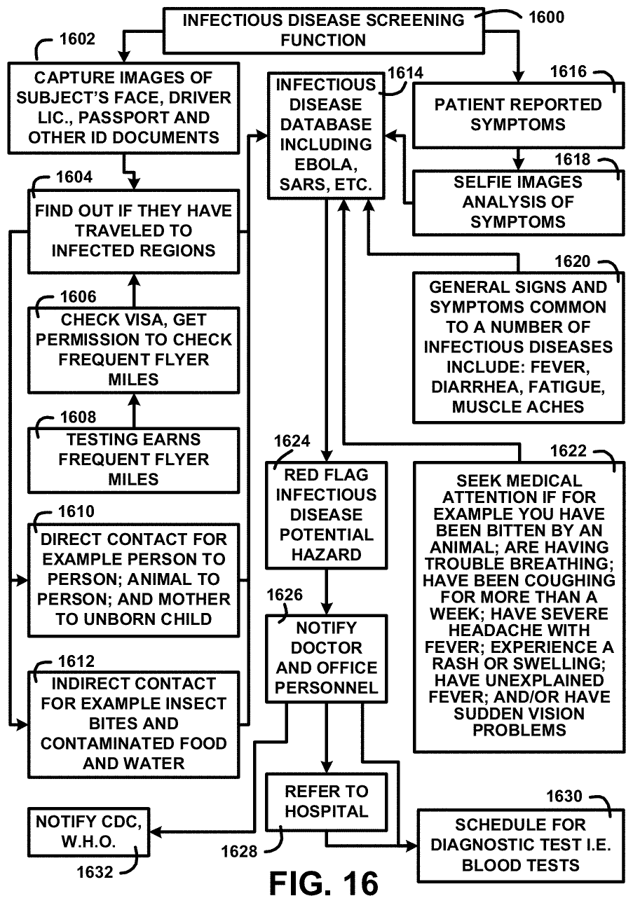

FIG. 16 shows a block diagram of an overview flow chart of infectious disease screening function of one embodiment. FIG. 16 shows a locked device function used for infectious disease screening function 1600. The locked device infectious disease screening function 1600 can for example capture images of subject's face, driver lic., passport and other id documents 1602.

The passport may for example be used to find out if they have traveled to infected regions 1604. Checking travel may include a process to check visa, get permission to check frequent flyer miles 1606. To encourage authorization to check frequent flyer miles accounts for potential exposure to an infectious disease a promotion can be offered through ad placement advertisers wherein testing earns frequent flyer miles 1608.

A questionnaire can be used to whether the person had direct contact for example person to person; animal to person; and mother to unborn child 1610 and/or indirect contact for example insect bites and contaminated food and water 1612. The integrated mobile device management service provider digital programmable server 100 can include an infectious disease database including Ebola, SARS, Zika Virus, etc. 1614. The locked device can be used to query patient reported symptoms 1616 from the infectious disease database including Ebola, SARS, Zika Virus, etc. 1614 to determine suspected exposures. A locked device camera can be used to capture images wherein a selfie images analysis of symptoms 1618 can be processed and queried from the infectious disease database including Ebola, SARS, Zika Virus, etc. 1614 to determine suspected exposures.

General signs and symptoms common to a number of infectious diseases include: fever, diarrhea, fatigue, muscle aches 1620. If symptoms and travel to infected regions indicate potential exposure to an infectious disease the person can be advised to seek medical attention if for example you have been bitten by an animal; are having trouble breathing; have been coughing for more than a week; have severe headache with fever; experience a rash or swelling; have unexplained fever; and/or have sudden vision problems 1622. Additional actions may include to red flag infectious disease potential hazard 1624, notify doctor and office personnel 1626, refer to hospital 1628, schedule for diagnostic test i.e. blood tests 1630, and notify CDC, W.H.O. 1632 of one embodiment.

The tablet coordinated care digital electronic platform can be used by a doctor when determining what lab test is appropriate for a particular patient. For example a doctor may feel based on past experience that a particular lab test might be useful in diagnosing a patient. However, under the Affordable Care Act a medical necessity for the lab test must be documented. The documentation of a medical necessity will include for example DNA test results showing the patient has a high risk of a particular cancer. The doctor can use the tablet coordinated care digital electronic platform during the patient examination to input a preliminary diagnosis to determine whether the preliminary diagnosis has documented support using an automatic review of the patient's medical records to determine whether the preliminary diagnosis is supported. If the preliminary diagnosis is supported the tablet coordinated care digital electronic platform will display a list of lab tests authorized by Medicare and the patient's health insurance carrier for that preliminary diagnosis. The doctor makes a selection of an authorized lab test and the tablet coordinated care digital electronic platform gathers the supporting documentation, prepares the letters of medical necessity, prepares the lab test requisition forms, schedules the lab test and displays the information to the doctor, who can pass the schedule lab test information along to the patient including a printed version the patient can pick up on their way out of the doctor's office.

This prevents the doctor from unintentionally ordering a lab test that is not authorized and that has been deemed inappropriate for the patient's condition. The doctor can rest assured that the appropriate lab test is being utilized thereby preventing the doctor from over use of an unwarranted test and under use of an appropriate test being performed. The tablet coordinated care digital electronic platform using a proprietary electronic lab requisition system and lab portal can be used to obtain fast and properly completed requisition forms, which greatly reduces incorrect and incomplete requisition forms.

A hospital clinical diagnostic laboratory test can be established using a reference lab through an “under an arrangement” made by the hospital or rural primary care hospital with reference laboratory. The “under an arrangement” is defined as a contractual agreement between the hospital and the reference laboratory under which the hospital will pay the reference laboratory for the tests performed and the hospital will be responsible for billing and collection activities. For outpatient hospital lab billing, the platform can be used to facilitate an electronic based outpatient outreach program for an outpatient hospital lab billing or Hospital Outpatient Department (HOPD). One such HOPD outreach program would be a behavioral mental health program between the HOPD and a remote doctor's office. A bona fide patient-hospital relationship is created when the remote outpatient visits the doctor's (offsite from the hospital), has a lab test done for the HOPD and takes the HOPD's remote computerized psychological test (CPT code 96103) and/or neuropsychological test (CPT code 96120). A bona fide outpatient/hospital relationship is also created when the remote outpatient takes the test at home or at a location other than the hospital as directed and/or prescribed by the HOPD and then has the lab test done at the doctor's office or via a mobile phlebotomist. The Federal Register/Vol. 79, No. 219/Thursday, Nov. 13, 2014 provides that “ . . . psychological testing services . . . (CPT codes 96103 and 96120) are not Medicare telehealth services as defined under the Act and thus can be furnished when beneficiary is not in the same place as the practitioner.” This means the HOPD can bill the lab work along with remote CPT codes 96103 and/or 96120, thus, creating a bona fide outpatient billable event for both the lab work and CPT code 96103 and/or CPT code 96120. This relationship between the HOPD and the outpatient is an integral part of the electronic operated outreach program.

Electronic Mental Health Assessment:

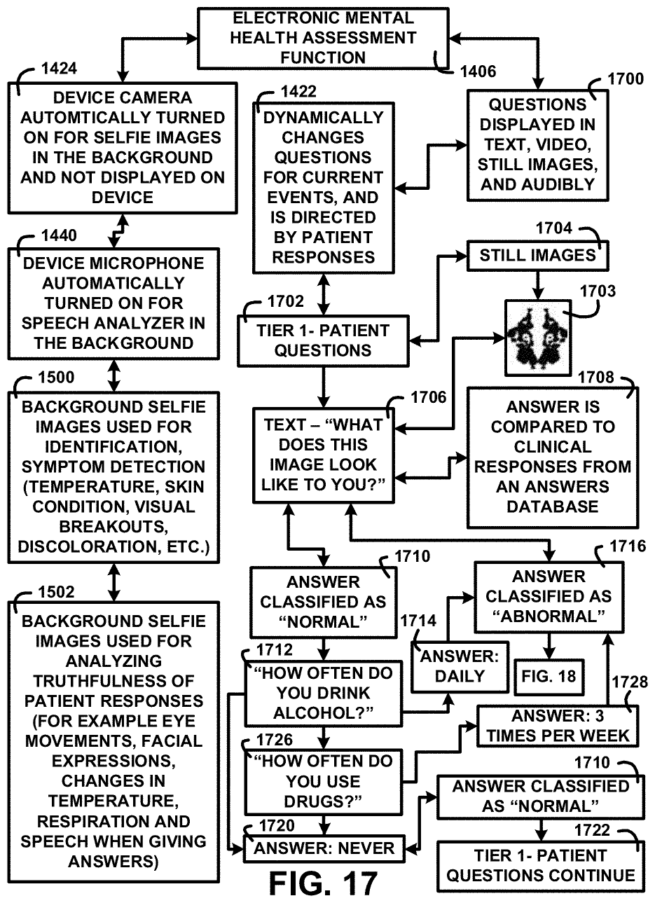

FIG. 17 shows a block diagram of an overview flow chart of electronic mental health assessment function of one embodiment. FIG. 17 shows a locked device electronic mental health assessment function 1406. A locked device camera is automatically turned on for selfie images in the background and not displayed on device 1424. A locked device microphone is automatically turned on for a speech analyzer in the background 1440. Background selfie images are used for identification, symptom detection (temperature, skin condition, visual breakouts, discoloration, etc.) 1500. Background selfie images are used for analyzing truthfulness of patient responses (for example eye movements, facial expressions, changes in temperature, respiration and speech when giving answers) 1502.

The electronic mental health assessment function 1406 can include a mental health patient progressive multi-level questionnaire 1402 of FIG. 14. The questionnaire can be configured to show questions displayed in text, video, still images, and audibly 1700. The electronic mental health assessment function 1406 dynamically changes questions for current events, and is directed by patient responses 1422. The mental health patient progressive multi-level questionnaire 1402 of FIG. 14 can include tiers of patient questions that progress through the tiers based on an analysis of patient responses.

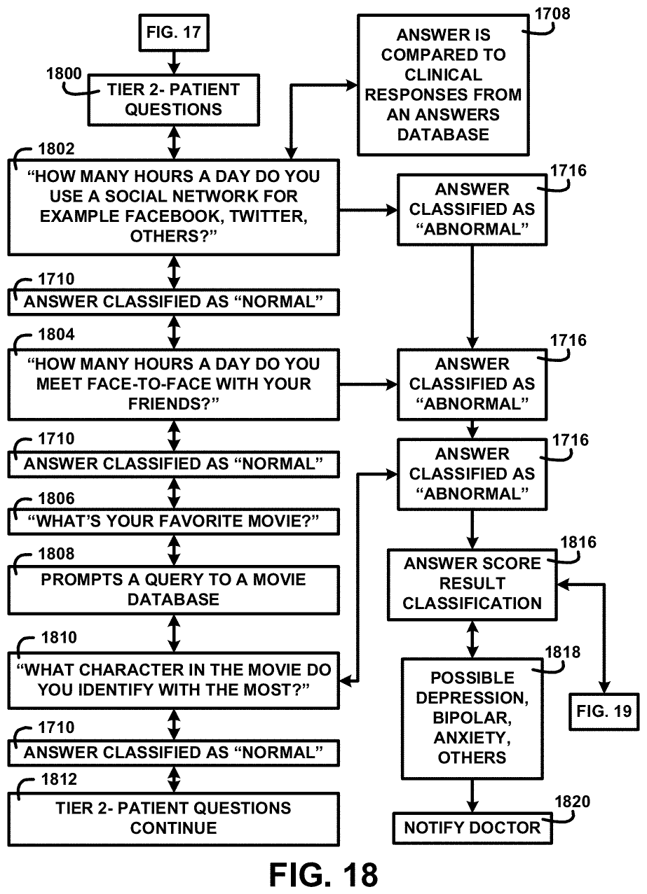

Tier 1-patient questions 1702 can include at least one still images 1704, at least one Rorschach test image 1703, and for example text—“what does this image look like to you?” 1706 questions. A patient answer compared to clinical responses from answers database 1708 is analyzed and a determination made with an answer classified as “normal” 1710. The questionnaire continues with “how often do you drink alcohol?” 1712 with a response answer: daily 1714 with answer classified as “abnormal” 1716 wherein the questionnaire is directed to a continuation shown in FIG. 18. A patient answer: never 1720 results in answer classified as “normal” 1710 wherein tier 1-patient questions continue 1722. The next tier 1 questions may be for example “how often do you use drugs?” 1726 wherein an answer: 3 times per week 1728 results in answer classified as “abnormal” 1716 wherein the questionnaire is directed to a continuation shown in FIG. 18. A patient answer: never 1720 results in answer classified as “normal” 1710 and tier 1-patient questions continue 1722 and so on of one embodiment.

Tier 2-Patient Questions:

FIG. 18 shows a block diagram of an overview flow chart of tier 2-patient questions of one embodiment. FIG. 18 shows a continuation from FIG. 17 showing tier 2-patient questions 1800. Answers to tier 2-patient questions 1800 are scored and recorded. Tier 2-patient questions 1800 include for example “how many hours a day do you use a social network for example Facebook, Twitter, others?” 1802. A patient answer is compared to clinical responses from an answers database 1708. An answer classified as “abnormal” 1716 is scored and recorded. An answer classified as “normal” 1710 continues the progress of the questionnaire. A next question “how many hours a day do you meet face-to-face with your friends?” 1804 may create an answer classified as “normal” 1710 or an answer classified as “abnormal” 1716.

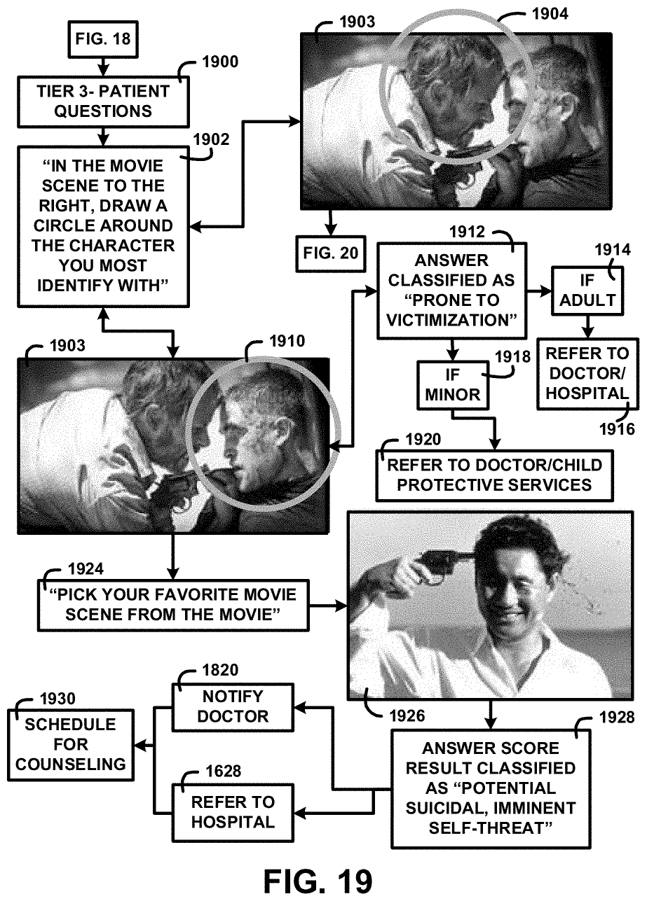

The questionnaire continues with “what's your favorite movie?” 1806, which prompts a query to a movie database 1808. Questions continue wherein the questions have dynamically changed to relate to the movie selected by the patient and movie information from the movie database. The next question by be for example “what character in the movie do you identify with the most?” 1810, patient responses may result in an answer classified as “normal” 1710 or an answer classified as “abnormal” 1716 and tier 2-patient questions continue 1812. As the questionnaire is progressing an answer score result classification 1816 is developed wherein the answer score result classification 1816 may indicate possible depression, bipolar, anxiety, others 1818 and an alert is sent to notify doctor 1820. The answer score result classification 1816 may redirect the questionnaire as shown in FIG. 19 of one embodiment.

Tier 3-Patient Questions:

FIG. 19 shows for illustrative purposes only an example of tier 3-patient questions of one embodiment. FIG. 19 shows a continuation from FIG. 18 to tier 3-patient questions 1900. The questionnaire continues with tier 3-patient questions 1900 referring to the patient movie selected with for example the following question that includes a text question, a still images and a patient interactive selection feature.

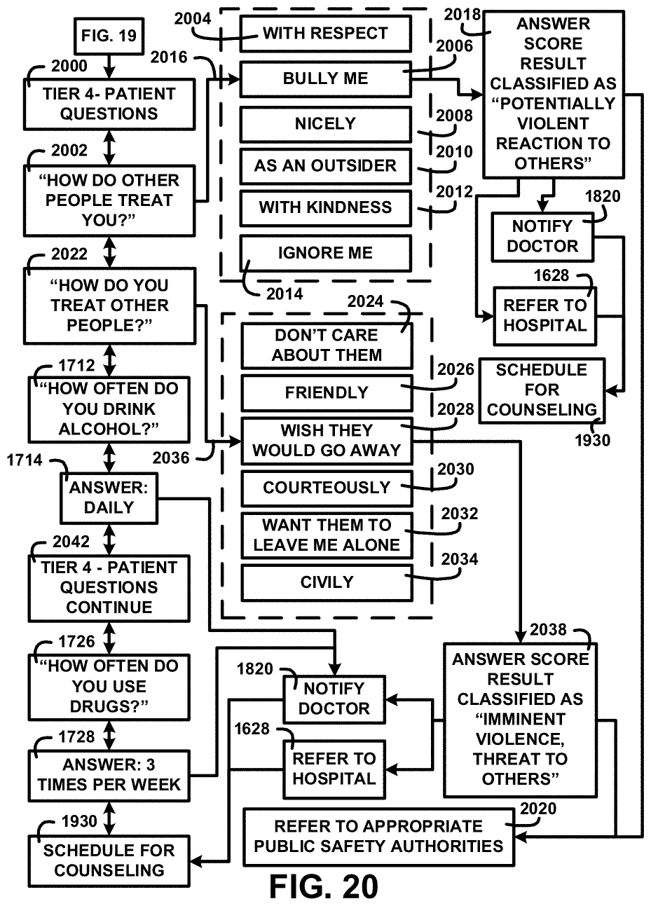

The question may be “in the movie scene to the right, draw a circle around the character you most identify with” 1902. The patient response may be where the patient circles the attacker 1904. This patient response redirects the questionnaire to continue as shown in FIG. 20. A patient response wherein the patient circles the victim 1910, may result in an answer classified as “prone to victimization” 1912. Follow-up actions if adult 1914 may include refer to doctor/hospital 1916, if minor 1918 may include refer to doctor/child protective services 1920.

The next question may be for example “pick your favorite movie scene from the movie” 1924. If the patient picks the suicide scene 1926 this answer score result classified as “potential suicidal, imminent self-threat” 1928 and prompt follow-up actions including for example notify doctor 1820, refer to hospital 1628 and/or schedule for counseling 1930 of one embodiment.

Tier 4-Patient Questions:

FIG. 20 shows a block diagram of an overview flow chart of tier 4-patient questions of one embodiment. FIG. 20 shows continuing from FIG. 19 are tier 4-patient questions 2000. Tier 4-patient questions 2000 can include for example “how do other people treat you?” 2002. The questions can include for example the following predetermined responses from which a patient will select their answer for example with respect 2004, bully me 2006, nicely 2008, as an outsider 2010, with kindness 2012, and ignore me 2014. A patient picks bully me 2016. This answer score result classified as “potentially violent reaction to others” 2018 prompting follow-up actions including for example notify doctor 1820, refer to hospital 1628, schedule for counseling 1930 and/or refer to appropriate public safety authorities 2020.

The next question “how do you treat other people?” 2022 may include for example the following predetermined responses including don't care about them 2024, friendly 2026, wish they would go away 2028, courteously 2030, want them to leave me alone 2032, and civilly 2034. A patient picks wish they would go away 2036, an answer score result classified as “imminent violence, threat to others” 2038. Follow-up actions include for example notify doctor 1820, refer to hospital 1628, schedule for counseling 1930, and/or refer to appropriate public safety authorities 2020.

Tier 4-patient questions continue 2042. The next question “how often do you drink alcohol?” 1712 receives an answer: daily 1714 with follow-up actions for example notify doctor 1820 and/or schedule for counseling 1930. Tier 4-patient questions continue 2042 with the next question “how often do you use drugs?” 1726 with a patient answer: 3 times per week 1728. The patient answer prompts follow-up actions for example notify doctor 1820 and/or schedule for counseling 1930 of one embodiment.

Substance Use and Mental Health in U.S. Adults:

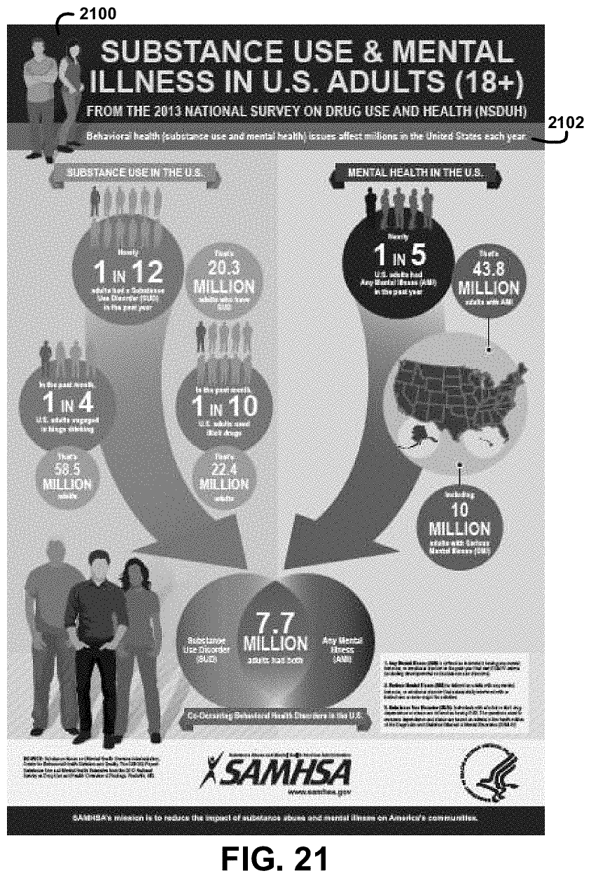

FIG. 21 shows for illustrative purposes only an example of substance use and mental health in U.S. adults of one embodiment. FIG. 21 shows a SUBSTANCE USE AND MENTAL HEALTH IN U.S. ADULTS (18+) poster ‘Substance Abuse and Mental Health Services Administration (SAMHSA)” 2100 which shows “behavioral health (substance use and mental health) issues affect millions in the united states each year” 2102. The poster and statistically data included shows a corollary between substance use and mental health conditions of one embodiment.

Locked Device Functionalities with Hands-Free Devices:

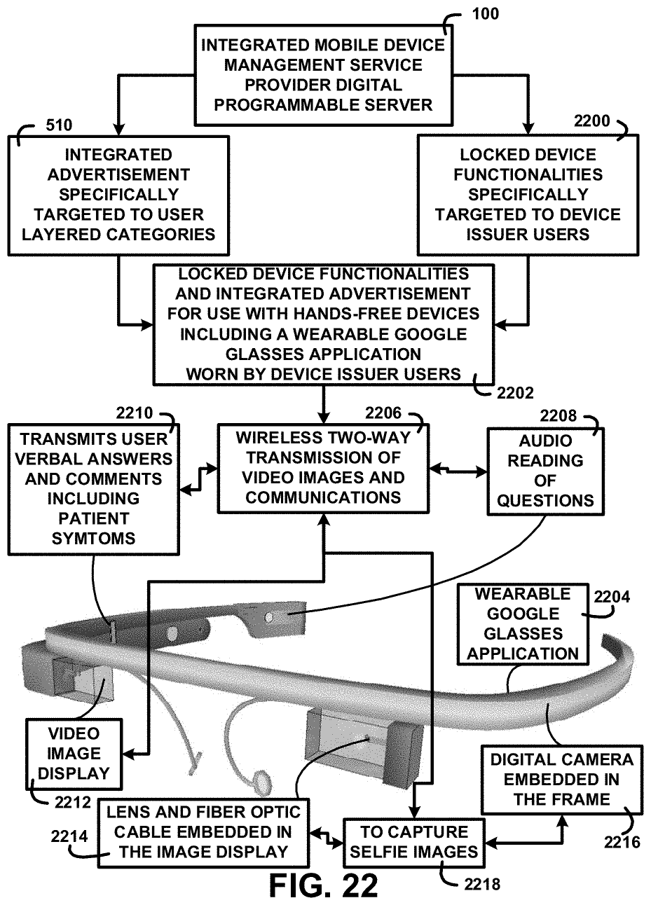

FIG. 22 shows for illustrative purposes only an example of locked device functionalities with hands-free devices of one embodiment. FIG. 22 shows the integrated mobile device management service provider digital programmable server 100 used to process locked device functionalities specifically targeted to device issuer users 2200 and integrated advertisement specifically targeted to user layered categories 510. FIG. 22 shows locked device functionalities and integrated advertisement for use with hands-free devices including google glasses worn by device issuer users 2202.

Google glasses 2204 may include locked device functionalities including wireless two-way transmission of video images and communications 2206, audio reading of questions 2208, transmits user verbal answers and comments including patient symptoms 2210, video image display 2212. Google glasses 2204 can include customized modifications for locked device functionalities including for example lens and fiber optic cable embedded in the image display 2214, at least one digital camera embedded in the frame 2216 to capture selfie images 2218 and wireless two-way transmission of video images and communications 2206 of one embodiment.

Biometric Devices User Identification Function:

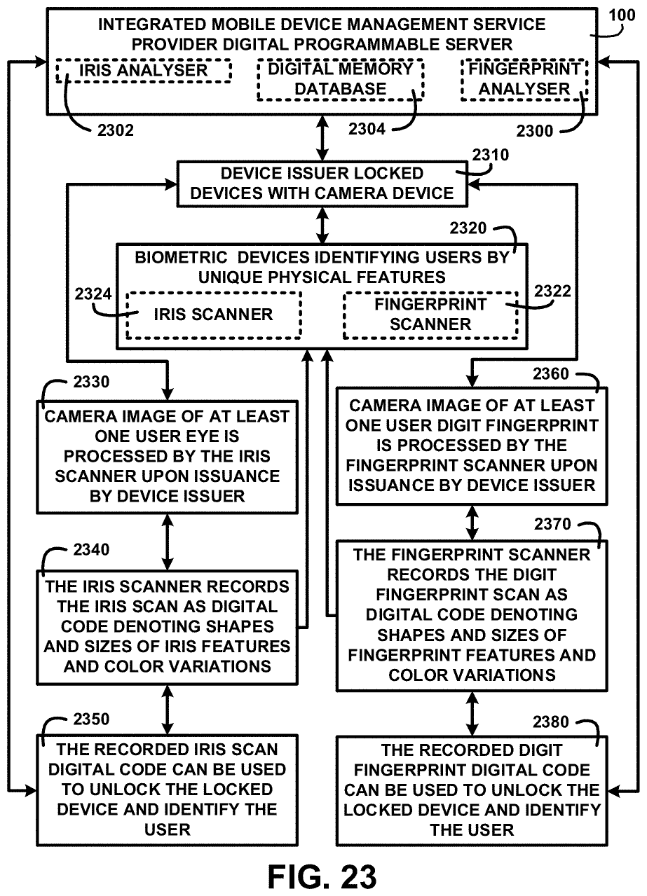

FIG. 23 shows a block diagram of an overview flow chart of biometric devices user identification function of one embodiment. FIG. 23 shows the integrated mobile device management service provider digital programmable server 100 controlling and processing device issuer locked devices biometric devices user identification function operations. Device issuer locked devices biometric devices user identification function operations include biometric devices identifying users by unique physical features 2320. One or more biometric devices identifying users by unique physical features 2320 include for example a fingerprint analyzer 2300, an iris analyzer 2302, one or more digital memory database 2304, and device issuer locked devices with camera device 2310.

Biometric devices identifying users by unique physical features 2320 can be configured to include a fingerprint scanner 2322 and an iris scanner 2324. A camera image of at least one user digit fingerprint is processed by the fingerprint scanner upon issuance by device issuer 2360. A fingerprint scanner records the digit fingerprint scan as digital code denoting shapes and sizes of fingerprint features and color variations 2370. The recorded digit fingerprint digital code can be used to unlock the locked device and identify the user 2380. A camera image of at least one user eye is processed by the iris scanner upon issuance by device issuer 2330. The iris scanner records the iris scan as digital code denoting shapes and sizes of iris features and color variations 2340. The recorded iris scan digital code can be used to unlock the locked device and identify the user 2350 of one embodiment.

Designated Locked Schedule and Locations Function:

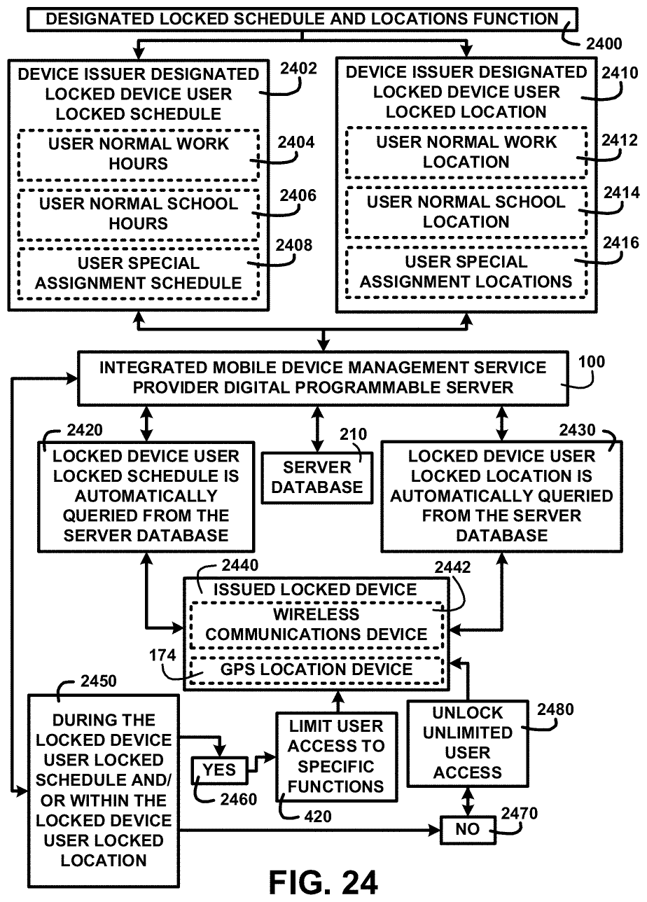

FIG. 24 shows a block diagram of an overview flow chart of designated locked schedule and locations function of one embodiment. FIG. 24 shows a designated locked schedule and locations function 2400 of the integrated mobile device management service provider digital programmable server 100. The designated locked schedule and locations function 2400 is used to record in the server database 210 a device issuer designated locked device user locked schedule 2402. The device issuer designated locked device user locked schedule 2402 includes for example a beginning time of day and an ending time of day for each day of the week. The device issuer designated locked device user locked schedule 2402 includes for example user normal work hours 2404, user normal school hours 2406, and a user special assignment schedule 2408.

The designated locked schedule and locations function 2400 is used to record in the server database 210 a device issuer designated locked device user locked location 2410. The device issuer designated locked device user locked location 2410 includes for example a physical boundary of a geographic region using GPS coordinates. The device issuer designated locked device user locked location 2410 includes for example a user normal work location 2412, a user normal school location 2414 and user special assignment locations 2416.

An issued locked device 2440 is configured with a wireless communications device 2442 including at least one WIFI device 178 of FIG. 1 and uses the wireless communications device 2442 to communicate with the integrated mobile device management service provider digital programmable server 100 and server database 210. The designated locked schedule and locations function 2400 performs a process wherein a locked device designated locked location is automatically queried from the server database 2430 and a locked device designated locked schedule is automatically queried from the server database 2420.

An issued locked device 2440 is configured with a GPS location device 174 to automatically transmit continually during the day the issued locked device 2440 current location GPS coordinates to the integrated mobile device management service provider digital programmable server 100 and server database 210. The integrated mobile device management service provider digital programmable server 100 uses an algorithm to determine whether the current location GPS coordinates of the issued locked device 2440 is within boundaries of the device issuer designated locked device user locked location 2410. An issued locked device 2440 is configured to automatically transmit continually during the day the issued locked device 2440 current time of day adjusted for the current time zone in which the issued locked device 2440 is located using a time adjustment algorithm.

The integrated mobile device management service provider digital programmable server 100 uses the results of the calculations performed by the one or more algorithms to determine if the issued locked device 2440 current adjusted time of day and current location is during the locked device user locked schedule and/or within the locked device user locked location 2450. If the determination result is yes 2460 the integrated mobile device management service provider digital programmable server 100 in one embodiment transmits electronically coded instructions to the issued locked device 2440 to continue to limit user access to specific functions 420.

If the determination result is no 2470 the integrated mobile device management service provider digital programmable server 100 in one embodiment transmits electronically coded instructions to the issued locked device 2440 to unlock unlimited user access 2480. Unlimited user access may occur for example during an issued locked device 2440 user's authorized vacation from work wherein the device issuer inputs into the server database 210 a locked device user locked schedule and locked device user locked location override designation to be effective during the issued locked device 2440 user's authorized vacation duration of one embodiment.

Home Care Automated Auditing System Functionality:

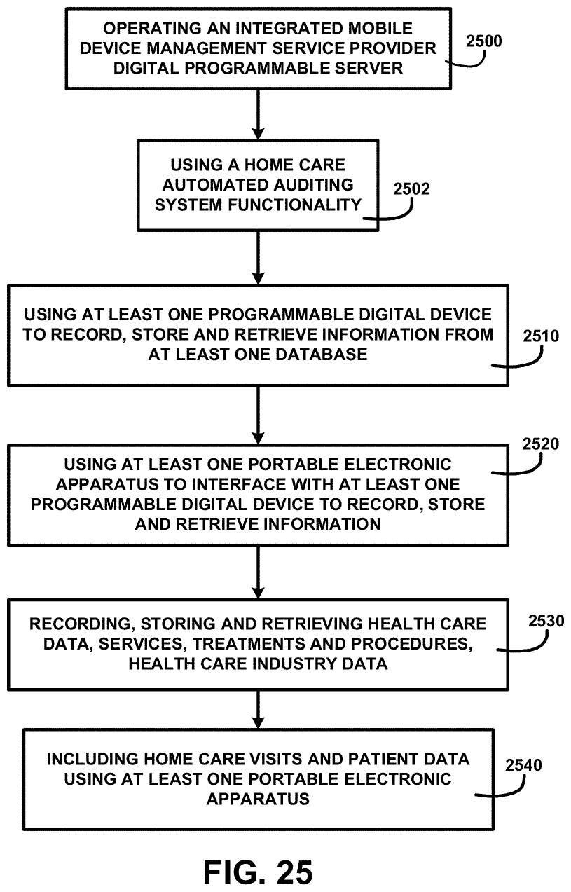

FIG. 25 shows a block diagram of overview of an integrated mobile device management system home care automated auditing system functionality of one embodiment. FIG. 25 shows operating an integrated mobile device management service provider digital programmable server 2500 using a home care automated auditing system functionality 2502 linked to the integrated mobile device management service provider digital programmable server 100 of FIG. 1. The home care automated auditing system functionality 2502 is using at least one programmable digital device to record, store and retrieve information from at least one database 2510 and using at least one portable electronic apparatus to interface with at least one programmable digital device to record, store and retrieve information 2520. The at least one programmable digital device is configured to include at least one functionality algorithm embedded processor to analyze and calculate at least one home care automated auditing system functionality 2502. The home care automated auditing system functionality 2502 is recording, storing and retrieving health care data, services, treatments and procedures, health care industry data 2530 including home care visits and patient data using at least one portable electronic apparatus 2540. The at least one portable electronic apparatus 2540 is configured to include at least one functionality algorithm embedded processor.

A Home Care Automated Auditing System:

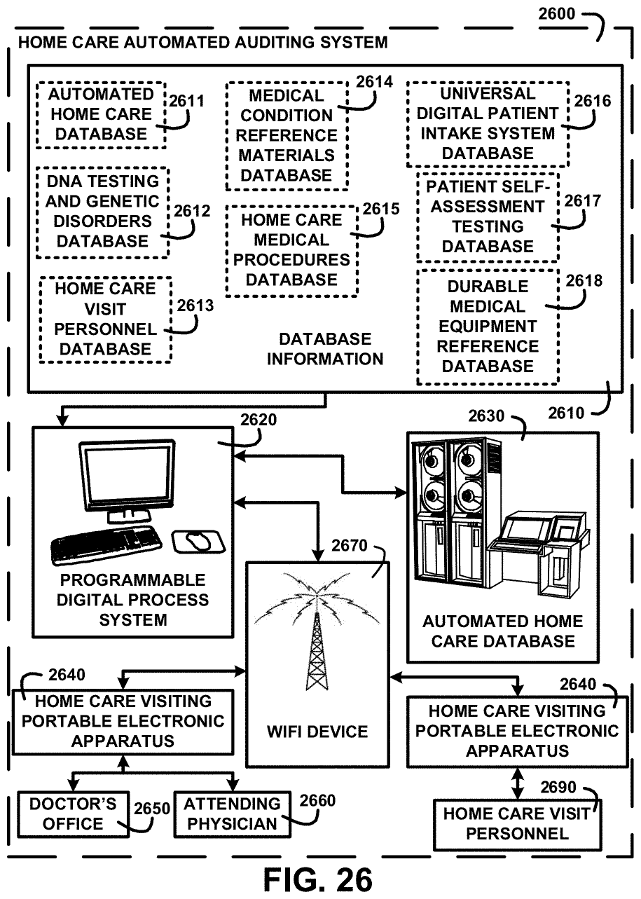

FIG. 26 shows for illustrative purposes only an example of a home care automated auditing system of one embodiment. FIG. 26 shows a home care automated auditing system 2600 including database information 2610, including for example an automated home care database 2611, DNA testing and genetic disorders database 2612, home care visit personnel database 2613, medical condition reference materials database 2614, home care medical procedures database 2615, universal digital patient intake system database 2616, patient self-assessment testing database 2617, and durable medical equipment reference database 2618.

The home care automated auditing system 2600 includes at least one programmable digital process system 2620 linked to an automated home care database 2611 and at least one WIFI device 2670. The at least one WIFI device 2670 is used to communicate to at least one home care visiting portable electronic apparatus 2640 used in a doctor's office 2650 and/or by an attending physician 2660. The at least one home care visiting portable electronic apparatus 2640 is one embodiment of the device issuer locked electronic devices 490 of FIG. 4. The at least one home care visiting portable electronic apparatus 2640 are configured to include at least one functionality algorithm embedded processor. The home care visiting portable electronic apparatus 2640 can also be used by home care visit personnel 2690 of one embodiment.

Automated Home Care Database:

The automated home care database 2630 is configured to receive and record from the attending physician 2660 data including a patient's name, address and other identification data, condition, prescriptions, and schedule of home care visits. The information can include patient universal automated patient intake system data and health history downloaded from a patient electronic device. The home care visits include visiting nurses, technicians, therapist, psychologist, psychiatrist and other licensed and certified professionals and trained personnel.

The automated home care database 2630 is configured to receive input including the name, address, telephonic contact numbers, texting contacts, license and certification, including issue and expiration dates of the home care visiting personnel that have been approved by insurance providers, attending physicians, local area hospitals and clinics, state and local licensing agencies.

The attending physicians will select home care visit personnel 2690 from the approved list. The automated home care database 2630 will automatically transmit and send to the home care visit personnel 2690 the patient's name, address and other identification data, condition, prescriptions, and schedule of home care visits and specific instructions from the attending physician 2660.

Medical Condition Reference Materials Database:

The automated home care database 2630 will maintain the medical condition reference materials database 2614 on various conditions for example reconstructive facial, dental conditions and post-operative conditions including reference images at various stages in the recovery, post-operative conditions that indicate for example infection, poor healing and other complications that would be used by the visiting home care professional to assess the patient's condition and where indicated immediately notify the attending physician 2660 of any complication.

Home Care Medical Procedures Database:

The home care medical procedures database 2615 is configured to include and maintain a database of reference materials on various procedures for example drawing blood, emergency resuscitation, cardiopulmonary resuscitation (CPR), Deoxyribonucleic acid (DNA) testing, drug testing and condition assessment factors that are checked to fully determine the patient's current condition.

Durable Medical Equipment Reference Database:

The automated home care database 2630 will maintain the durable medical equipment reference database 2618 (DME). The DME reference materials include operating instructions, photos of the equipment and image details to facilitate a patient's use of the equipment. Additionally included are checklist on the condition of the equipment to assure that proper maintenance is being performed and the proper maintenance performance instructions for use by both patients and for example visiting nurses and technicians. Durable medical equipment (DME) includes for example iron lungs, oxygen tents, Nebulizers, CPAP, catheters, hospital beds, and wheelchairs.

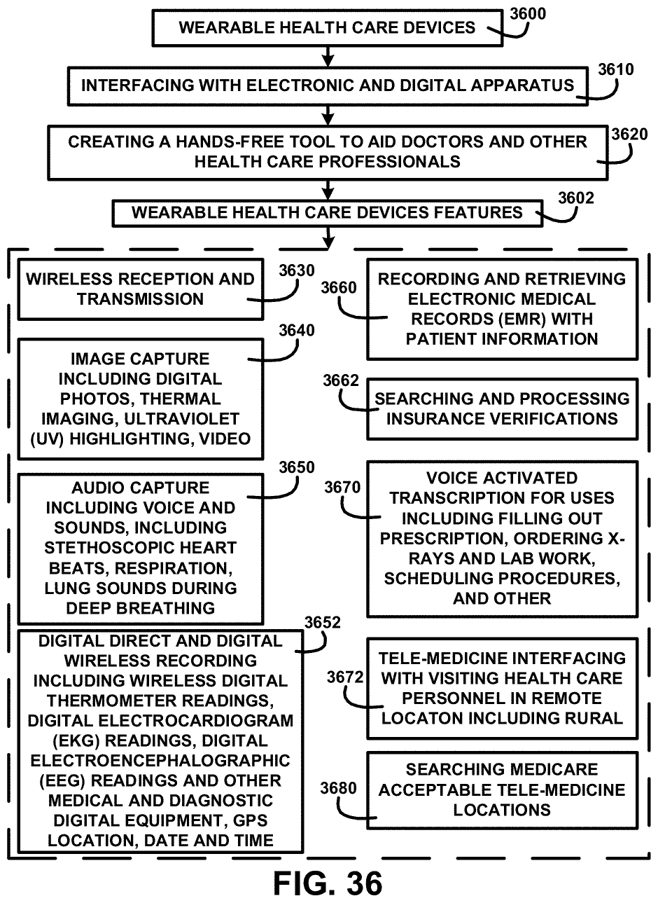

DME can be displayed in integrated advertisement ad placement areas on the device issuer locked electronic devices 490 of FIG. 4 including the home care visiting portable electronic apparatus 2640 of FIG. 26, and wearable health care devices 3600 of FIG. 36. Many of these types of DME are operated by a patient at home. The setup and operation can be confusing to an un-informed untrained patient. The home care automated auditing system 2600 provides the means of providing the patient with information and training to enable them to properly use the DME and receive the proper treatment benefits that come from using the DME.

In the following example a physician will have prescribed OPAP equipment to a patient diagnosed with sleep apnea. The Physician will select from a list of authorized technicians trained in the setup, operation and maintenance of the make and model of OPAP equipment prescribed. The durable medical equipment reference database 2618 will record and maintain a current list of authorized technicians in the local area. The selected technician will be scheduled for a home care visit with the patient to instruct and assist the patient in setting up the OPAP device, its operation and maintenance care. The technician will arrive at the patients home as scheduled. The technician's arrival will be recorded by the home care visiting portable electronic apparatus 2640. The technician may be delivering the device or the device may have been shipped to the location previously. The technician will unpack the device checking the manifest of parts to insure all parts have been provided. The manifest of parts is available on the home care visiting portable electronic apparatus 2640 using the durable medical equipment reference database 2618. The technician will check off by touching the part name and image shown on the home care visiting portable electronic apparatus 2640. The completeness of the device will be recorded and scored by the durable medical equipment reference database 2618 entries.

Prior to beginning the setup the technician will ask the patient to take a very brief self-assessment test using the home care visiting portable electronic apparatus 2640, to indicate the current level of understanding of the OPAP device, its purpose in the patient's treatment and the familiarity with the device and questions configured to measure the patient's anxiety, fear or other emotional issues dealing with the use of the OPAP device. This first test will be recorded in the patient self-assessment testing database 2617.

The durable medical equipment reference database 2618 will display on the home care visiting portable electronic apparatus 2640 detailed instruction of setting up the OPAP device. The technician can show the instructions displayed to the patient and explain the setup steps to make sure the patient is aware of the arrangement of parts and explain the parts purpose as indicated on the information displayed. The durable medical equipment reference database 2618 will show explanations that are worked for various technical levels and layman's terms to provide an adequate and complete understanding to the patient. Should a patient not read of understand English the technician can select a language from categories displayed on the home care visiting portable electronic apparatus 2640. The database will perform translations of the information in the language of choice of the patient. As the steps of the setup are complete and the information is conveyed to the patient the technician will ask the patient to select a touch screen area to indicate that the patient fully understands the step and part purpose, or that they need additional explanation to more fully understand, at which point the durable medical equipment reference database 2618 will display additional explanations and illustrative images to assist the technician is another approach to allow the patient to fully understand the step. Once the setup is complete the technician will indicate that completion on the home care visiting portable electronic apparatus 2640 and the durable medical equipment reference database 2618 will record the time taken and score that against an average and score the subsets of time and patient understanding that have been recorded during the setup.

The technician will proceed into the instruction of how to use the OPAP device. The durable medical equipment reference database 2618 will display the various conFIGurations of the usage of the device. The technician will show to the patient each conFIGuration and explain how the device operates under that conFIGuration and what the patient will have to do to reach that conFIGuration and start the operation of the OPAP device. At the end of each conFIGuration section the patient will select their level of understanding and the process will be repeated until the patient reaches a full understanding. These section completions will be scored and recorded on the durable medical equipment reference database 2618. Once the operational conFIGurations have been reviewed with each step having been scored a final score will be recorded.

The technician will then proceed to review the maintenance procedures with the patient. The durable medical equipment reference database 2618 will display on the home care visiting portable electronic apparatus 2640 the maintenance steps and frequencies with which the steps need to be performed to maintain the OPAP device in proper working order. The patient will select their level of understanding as each maintenance step as it is reviewed. Upon completion of each step a score will be computed and when all complete an overall score will be recorded as well.

During the explanations and instruction review the technician will ask the patient to perform each step on their own so that the patient becomes familiar with each part and receives a hands-on experience to belay any anxiety or fear of “missing things up”. The technician can record videos of the patient hands-on experience using the home care visiting portable electronic apparatus 2640.

At the completion of the instructions the technician will ask the patient to take a very brief second self-assessment test using the home care visiting portable electronic apparatus 2640, to indicate the patient level of understanding of the OPAP device after the instructions to measure the effectiveness of the instructions and the technician's communication of the same. The second test will include questions configured to re-measure the patient's anxiety, fear or other emotional issues dealing with the use of the OPAP device. This second test will be recorded in the patient self-assessment testing database 2617 and a comparative score will be computed to measure the effectiveness of the technician's efforts and whether the patient may require additional sessions going forward. The departure of the technician will be recorded in the automated home care database 2630.

The scores of the steps will be arranged in a format to show the physician how the patient reacted to the DME, their understanding of it use and their forecasted ability to properly maintain the equipment. This formatted information can be reviewed in detail by the physician and for example the videos of the patient hands-on experience can enable the physician to evaluate his perception of the patient they are familiar with to look for visual indication on whether the patient is actually comfortable with the use of the device or whether the patients facial expressions and body language indicate probable follow-up sessions will be necessary to get the patient comfortable with its use. This ability to better predict patient proper use can prevent the patient from not using the equipment at all or improperly using the equipment and further complicating their condition. The DME auditing provided by the home care automated auditing system 2600 enables a physician to realistically ascertain the effective treatment being provided by the DME and provide early intervention to assist the patient in receiving the treatment possible by using the DME in a comfortable and proper manner.

Patient Self-Assessment Testing Database:

The automated home care database 2630 will maintain the patient self-assessment testing database 2617 wherein condition assessment factor procedures are configured to include patient self-assessment tests. Patient self-assessment tests are configured to include direct patient input including their selection of general health conditions and difficulties, and tests that are used to determine the patient's emotional and psychological state. The patient will use a home care visiting professional's portable electronic apparatus to take the test. The test can be configured to include, check boxes, multiple choice or selections of pictorial representations. The patient self-assessment tests are used to assess the patient's mood, anxiety, worries, and location of pain on their body.

Tests are configured to include testing methods which are appropriate to the patient's condition, physical limitations, native language and current cognitive condition. The patient self-assessment testing database 2617 is configured to include patient self-assessment tests in at least one language and can include multiple languages suitable to the patient demographics in a local area for example Spanish in Hispanic populations, in populations of Asians the languages can include Chinese dialects, Vietnamese, Japanese, Korean and other Asian languages. The test can include testing methods that include audio files that read the question to the patient in their selected native tongue and record the patient's verbal response, and create a machine translation into English for the home care visiting professional to check and is unsure re-question the patient on their response and make additional inquiries to fully assess the patient's response and current condition.

Visiting Portable Electronic Apparatus:

The home care visit personnel 2690 including for example a nurse will use the home care visiting portable electronic apparatus 2640 including an electronic tablet, iPad, iPod or other digitally programmable device to record the visit. The patient's name, address and other identification data, condition, prescriptions, attending physician's instruction and other pertinent and vital information will be automatically downloaded from a database into the home care visiting portable electronic apparatus 2640. The visiting nurse's scheduled visits will also be downloaded into the apparatus.

The home care visiting portable electronic apparatus 2640 will automatically log in the physical visit information including global positioning system (GPS) location, date and time of day of arrival and departure date and time of day. Should a visiting nurse not be at the GPS location at the scheduled visit time, including not show a then current GPS location not within a reasonable distance from the patients GPS location to be able to reach the patient by the schedule appointment time the automated home care database 2630 will automatically send a text message to the visiting nurse to remind them of the appointment and request a response on when they will be able to arrive at the patients location. The automated home care database 2630 will automatically dial and contact the patient to notify the patient of the revised estimated time of arrival (ETA) of the visiting nurse.

The home care automated auditing system 2600 automatically stores the home care visit upon completion, patient input, answers to the questions, date, time and duration of the visit and performs a query on the billing information including coverage codes for services, and preparation of the invoicing to the insurance carrier to allow for immediate billing without the delay and cost of staff time.

A Universal Automated Patient Intake System:

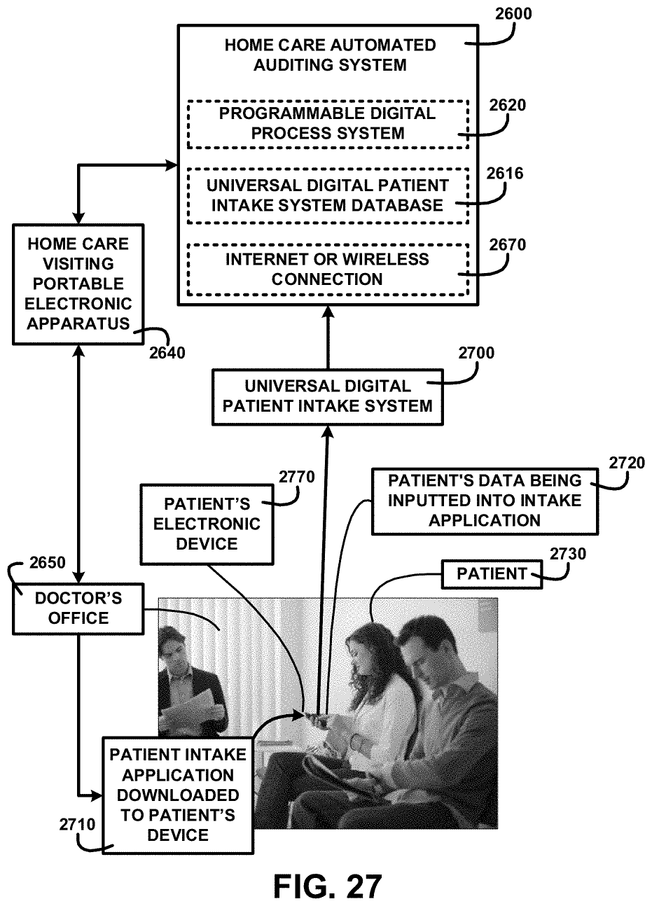

FIG. 27 shows for illustrative purposes only an example of a universal automated patient intake system of one embodiment. FIG. 27 shows the patient 2730 in the doctor's office 2650. The home care automated auditing system 2600 includes a programmable digital process system 2620, universal digital patient intake system database 2616 and Internet or wireless connection 2670. A doctor's office 2650 staff member can use the home care visiting portable electronic apparatus 2640 to process the patient intake application downloaded to patient's device 2710. The patient 2730 can then use a patient's electronic device 2770 for patient's data being inputted into intake application 2720 directly by the patient 2730 of one embodiment.

A Health Insurance Coverage Predetermination Database:

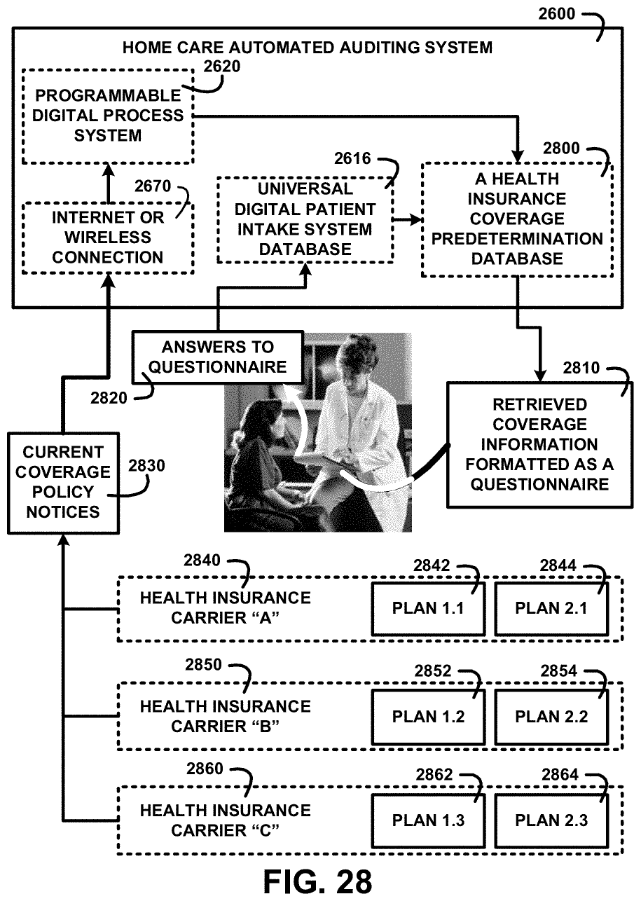

FIG. 28 shows for illustrative purposes only an example of a health insurance coverage predetermination database of one embodiment. FIG. 28 shows the home care automated auditing system 2600 including the programmable digital process system 2620, the universal digital patient intake system database 2616 providing health insurance providers, and a health insurance coverage predetermination database 2800. Information gathered from the health insurance coverage predetermination database 2800 provides retrieved coverage information formatted as a questionnaire 2810. The patient provides answers to questionnaire 2820 in making a health insurance coverage predetermination. The health insurance coverage predetermination database 2800 is used in a health insurance coverage predetermination process to determine for example whether a patient's health insurance coverage will pay for example for a specific lab test or treatment program. The process includes a static check using for example a patient's EMR, personal medical history, and current diagnostic results to search the patient's health insurance plan coverage to make an initial predetermination whether the example lab test or treatment program is covered and any payment limitations. The health insurance coverage predetermination process proceeds to make a dynamic check to predetermine whether the patient's health insurance plan coverage contains any qualifying conditions that may limit coverage. For example, the health insurance coverage predetermination process search results find the health insurance plan coverage contains a qualifying condition that a patient with a history of drug abuse would be denied coverage for an addictive drug treatment. The health insurance coverage predetermination process performs automatically to search the patient's EMR to determine whether the patient in the past has received treatment of drug abuse, disclosed in previous records any drug abuse, and a thorough search of available records for any indications of previous or current drug abuse. Should a doctor feel a treatment program will include a possibly addictive drug treatment, the health insurance coverage predetermination process will automatically alert the doctor that the health insurance plan coverage may not pay for this type of treatment. The doctor may then inform the patient that that specific treatment program if followed would not be paid for by the health insurance plan and the patient would have to make payment arrangements. If the health insurance coverage predetermination process does not find any qualifying condition a report is provided to the doctor that the health insurance plan coverage will pay for the specific treatment program.

The tablet coordinated care digital electronic platform can be used for electronic pre-qualification. Medical necessity is a required and necessary component of most medical procedures. Medical necessity is usually a part of the claim associated with the medical procedures. The tablet coordinated care digital electronic platform can be used by a doctor for pre-qualifying patients electronically and automatically for medical necessity against any medical plan and/or medical policy of the patient in real-time, as long as the appropriate medical criteria medical plan/policy is entered into the database. In addition, the electronic pre-qualification system can be used to control utilization by confirming medical necessity; and when medical necessity exists, automatically generate any required letter of medical necessity, according to a designated plan's medical criteria.

Current coverage policy notices 2830 from for example health insurance carrier “A” 2840 for plan 1.1 2842 and plan 2.1 2844, health insurance carrier “B” 2850 for plan 1.2 2852 and plan 2.2 2854 and health insurance carrier “C” 2860 for plan 1.3 2862 and plan 2.3 2864 is gathered using the Internet or wireless connection 2670. The updated current coverage policy notices 2830 is then stored in the health insurance coverage predetermination database 2800 as an example of the updating process of one embodiment.

A Medical Condition Reference Materials Database:

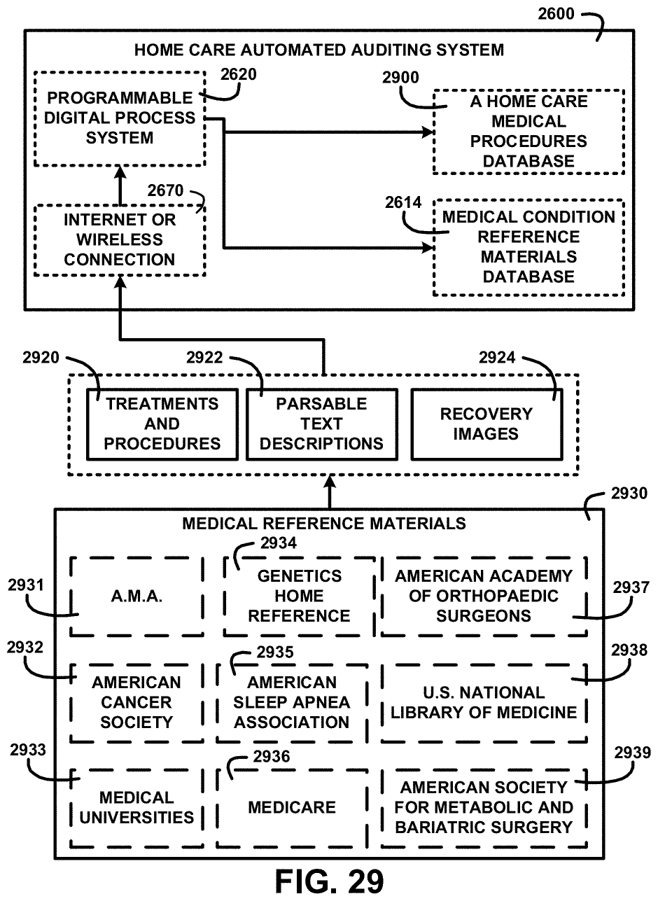

FIG. 29 shows a block diagram of a medical condition reference materials database of one embodiment. FIG. 29 shows the home care automated auditing system 2600 including the programmable digital process system 2620, a home care medical procedures database 2866, the medical condition reference materials database 2614, and the Internet or wireless connection 2670. A medical condition reference materials database in the home care automated auditing system 2600 includes treatments and procedures 2920, parsable text descriptions 2922 and recovery images 2924. An automated system update is constantly being performed for various medical reference materials 2930. The medical reference materials 2930 are gathered and updated from sources including A.M.A. 2931, American Cancer Society 2932, medical universities 2933, Genetics Home Reference 2934, American Sleep Apnea Association 2935, Medicare 2936, American Academy of Orthopedic Surgeons 2937, U.S. national library of medicine 2938, and American Society for Metabolic and Bariatric Surgery 2939 of one embodiment.

A Durable Medical Equipment Reference Database:

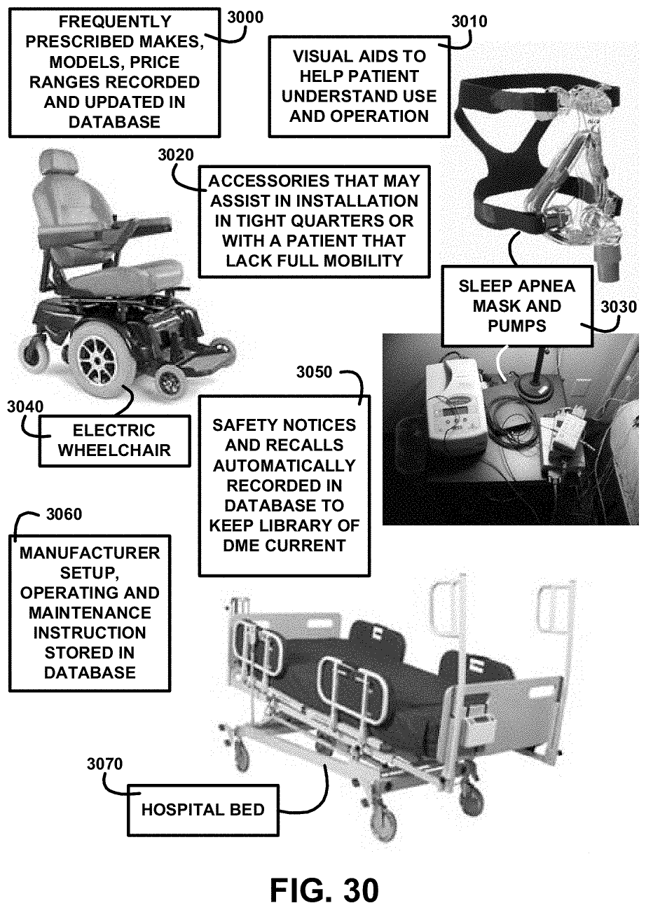

FIG. 30 shows for illustrative purposes only an example of a durable medical equipment reference database of one embodiment. FIG. 30 shows a durable medical equipment reference database including frequently prescribed makes, models, price ranges recorded and updated in database 3000. The database can be used to show patient visual aids to help patient understand use and operation 3010 of their prescribed DME. The database can be used to show patient accessories that may assist in installation in tight quarters or with a patient that lack full mobility 3020. The database can be used to show a patient various Sleep Apnea masks and pumps 3030.

The durable medical equipment reference database is automatically updated with safety notices and recalls automatically recorded in database to keep a library of DME current 3040.

The database can be used to show a patient one or more model of electric wheelchair 3050 and/or hospital bed 3070. The durable medical equipment reference database is automatically updated with current information on manufacturer setup, operating and maintenance instruction stored in database 3060 of one embodiment.

A Patient Self-Assessment Testing Database:

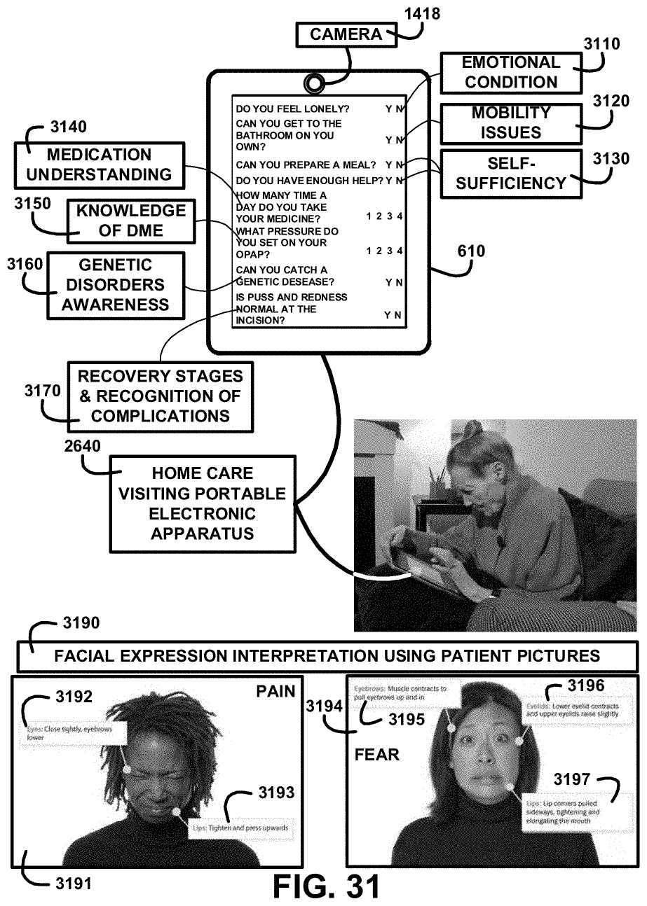

FIG. 31 shows for illustrative purposes only an example of a patient self-assessment testing database of one embodiment. FIG. 31 shows an electronic tablet 610 equipped with a camera 1418. The electronic tablet 610 can be used patient self-assessment testing. The camera 1418 can be used for capturing images of the patient while taking the self-assessment test. The self-assessment testing questions are downloaded from a patient self-assessment testing database in the integrated mobile device management service provider digital programmable server 100.

The questions are selected from the database based on the physical, mental and emotional state of the patient. For example questions can be used to assess a patient's emotional condition 3110 for example “do you feel lonely?” Y N. For example questions can be used to assess a patient's mobility issues 3120 for example “can you get to the bathroom on you own?” Y N. For example questions can be used to assess a patient's self-sufficiency 3130 for example “can you prepare a meal?” Y N and “do you have enough help?” Y N.

For example questions can be used to assess a patient's medication understanding 3140 for example “how many times a day do you take your medicine?” 1 2 3 4. For example questions can be used to assess a patient's knowledge of DME 3150 for example “what pressure do you set on your OPAP?” 1 2 3 4. For example questions can be used to assess a patient's genetic disorders awareness 3160 for example “can you catch a genetic disease?” Y N.

For example questions can be used to assess a patient's recovery stages & recognition of complications 3170 for example “is puss and redness normal at the incision?” Y N. The home care visiting portable electronic apparatus 2640 can be configured as the electronic tablet 610. The images of the patient captured while taking the self-assessment test can be used for facial expression interpretation using patient pictures 3190.

The facial expression interpretation may be signs of pain 3191 for example eyes: close tightly, eyebrows lower 3192 and lips: tighten and press upwards 3193 even though the patient is indicating in his or her answer they have no pain. The patient may act as though they have no worries or problems while their facial expression interpretation show signs of fear 3194 for example eyebrows: muscle contracts to pull eyebrows up and in 3195, eyelids: lower eyelid contracts and upper eyelids raise slightly 3196 and lips: lip corners pulled sideways, tightening and elongating the mouth 3197.

Inconsistencies between the patient's testing answers and the facial expression interpretations may prompt the physician to order more frequent unannounced visits to determine whether the patient may be in jeopardy but is afraid to say so for some unknown reason. The additional follow-up may uncover the reason and thereby allow the health care providers to find a solution to better assist the patient of one embodiment.

A Home Care Visiting Portable Electronic Apparatus Application:

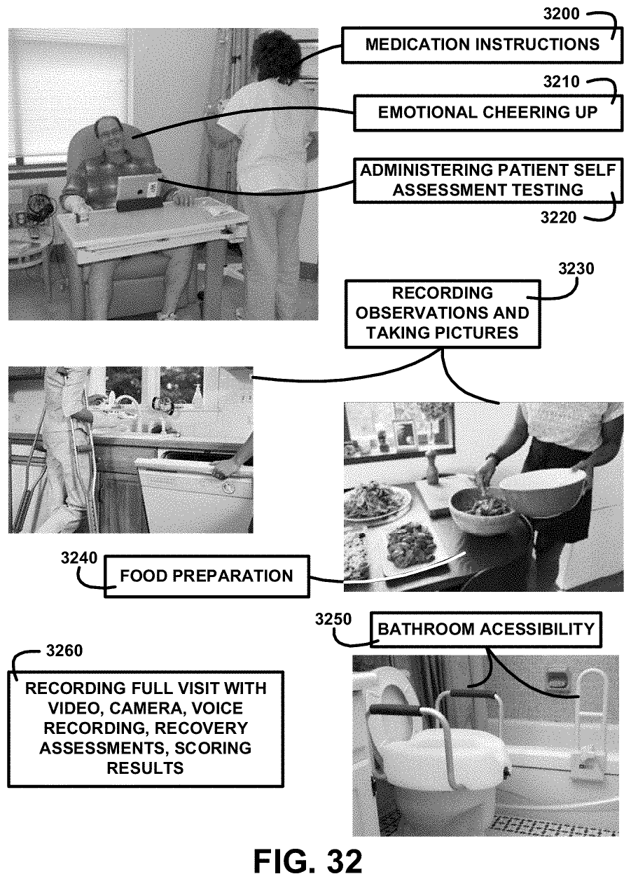

FIG. 32 shows for illustrative purposes only an example of a home care visiting portable electronic apparatus application of one embodiment. FIG. 32 shows a home care visiting portable electronic apparatus application being used to provide medication instructions 3200. The homecare visit and interaction with the homecare provider can provide emotional cheering up 3210 which for example for shut-in patients and/or patients with limited personal support.

The home care visiting portable electronic apparatus application can be used for administering patient self-assessment testing 3220 where the patient provides their impressions and comments of their own condition and/or recovery. The home care visiting portable electronic apparatus application can be used for recording observations and taking pictures 3230 of tasks the patient is performing for example cleaning dishes, and food preparation 3240. The observations may also include any personal assistance being provided by friends and family.

Recording pictures of bathroom accessibility 3250 is used to determine whether disabled equipment is available and properly installed or not and may prompt the ordering of accessibility equipment to assist the patient in bathroom use. The home care visiting portable electronic apparatus application can be used for recording full visit with video, camera, voice recording, recovery assessments, scoring results 3260 to enable the tending physician to determine the level of care the patient is receiving at home and order additional assistance as deemed in the patient's best interest of one embodiment.

Home Care Visit Nursing Auditing:

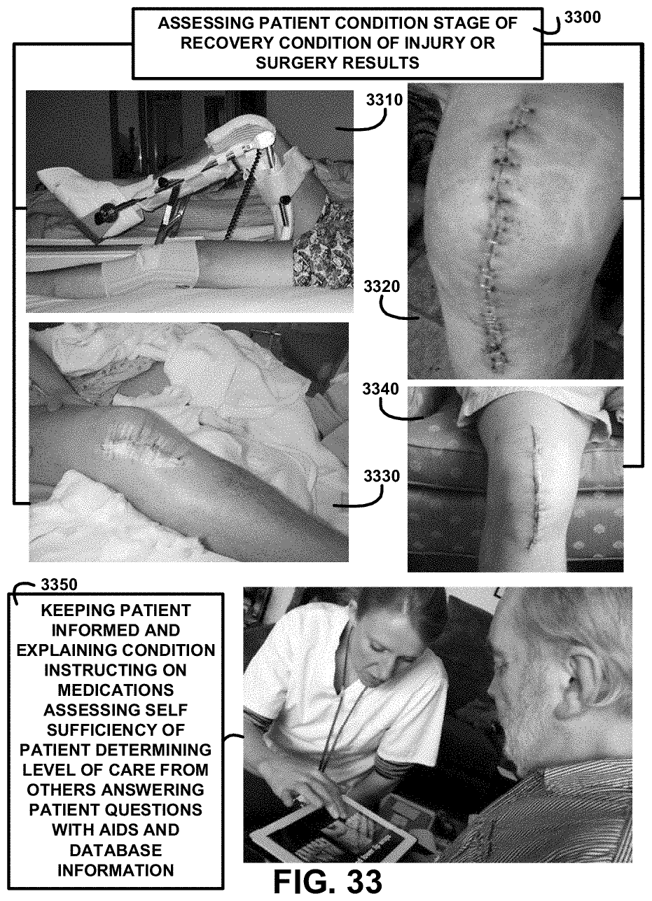

FIG. 33 shows for illustrative purposes only an example of home care visit nursing auditing of one embodiment. FIG. 33 shows home care visit nursing auditing being used for assessing patient condition stage of recovery condition of injury or surgery results 3300. Patient condition stages can include for example limb stabilization condition 3310, image of surgical stapled closure condition 3320, image of Steri-Strips closure condition 3330 and progressive condition of surgical closure 3340. Assessing patient condition stage of recovery is used for keeping patient informed and explaining condition instructing on medications assessing self-sufficiency of patient determining level of care from others answering patient questions with aids and database information 3350 of one embodiment.

The homecare provider using at least one programmable digital device can capture images of the post-surgical area condition and record those images in the integrated mobile device management service provider digital programmable server 100 of FIG. 1 patient EMR. The physician can review the captured images and make additional treatment arrangements or change or continue the current treatment regimen.

Home Care Visit DNA Testing Auditing:

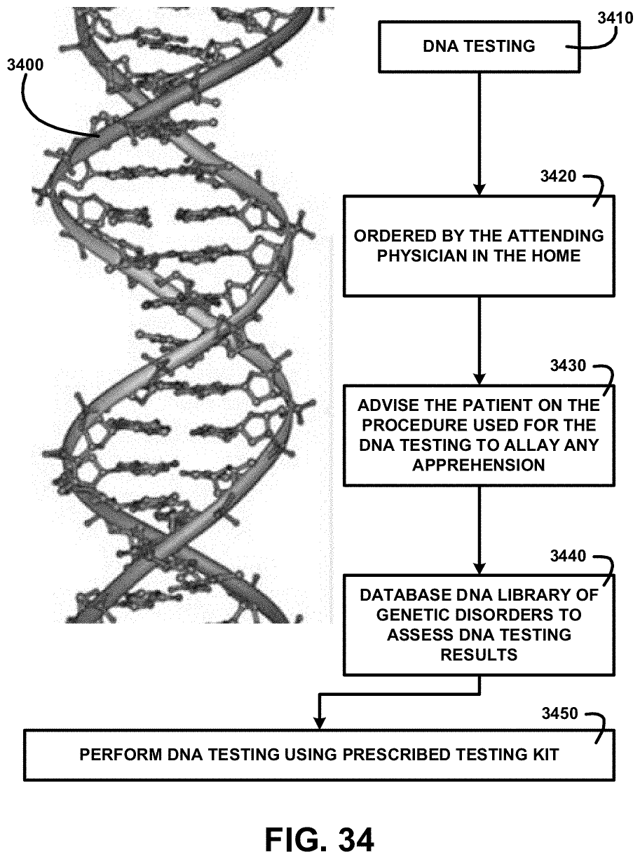

FIG. 34 shows for illustrative purposes only an example of home care visit DNA testing auditing of one embodiment. FIG. 34 shows an image of a DNA helix 3400 displayed on at least one programmable digital device used by a home care provider to explain DNA to a homecare patient and the reasons for DNA testing. DNA testing 3410 ordered by the attending physician in the home 3420 can be used to check the homecare patient for genetic disorders and predispositions to various conditions. The home care provider using the at least one programmable digital device can advise the patient on the procedure used for the DNA testing to allay any apprehension 3430. The integrated mobile device management service provider digital programmable server 100 of FIG. 1 includes a DNA testing and genetic disorders database 2612 including a DNA library of genetic disorders to assess DNA testing results 3440. The homecare provider can perform DNA testing using prescribed testing kit 3450 ordered by the physician thereby saving the patient an office visit.

The home care automated auditing system 2600 can include a DNA testing genetic polymorphism on drug disposition and response database herein the DNA testing and genetic disorders database 2612. The development of genetic testing and correlations between identified genotypes and patient disposition to negative responses to specific drugs has increased. The use of genetic testing can be used for the patient population that may have a genetic risk factor to certain drug severe drug reactions. The findings of the correlations are currently classified in two groups with the first group being Established Evidence-Based Clinical Guidelines and the second group Drug-Gene Associations Requiring Further Investigation.

Established Evidence-Based Clinical Guidelines currently are available for medical conditions including cardiovascular, pain, gastric, psychiatric, anti-fungal infection and transplantation. Drug-gene associations requiring further investigation have been preliminarily found for medical conditions including additional correlations for cardiovascular, pain, psychiatric and new correlations for anti-diabetic medications.

The DNA testing and genetic disorders database 2612 can be accessed through the home care automated auditing system 2600 using the home care visiting portable electronic apparatus 2640 including an electronic tablet, iPad, iPod, hands-free wearable health care devices or other digitally programmable device on which the physician has downloaded the application available from a subscription to the home care automated auditing system 2600. The DNA testing and genetic disorders database 2612 information can be used to assist health care providers in determining proper prescribing medications.

Although the DNA testing and genetic disorders database 2612 information is not a substitute for clinical and therapeutic drug monitoring and a physician's diagnosis and treatment based drug ordering determinations it does provide an additional level of alerting a physician to possible adverse drug reactions (ADRs). Adverse drug reactions occur even though the doctor wrote the correct prescription and the patient took the medication properly. Over 100,000 Americans die annually from ADRs. Many of the most serious drug reactions are also the most preventable. The DNA testing and genetic disorders database 2612 information will be automatically updated as new information becomes available from reliable sources.

A provider can perform a simple DNA test whose results can provide valuable insight into your patients’ reactions to medication(s) and their likelihood of developing common cardiovascular/thrombotic disorders. These DNA panels also tests for common genetic variants that cause thrombophilia or predict cardiovascular disease. Indications for DNA testing should be considered for patients for example going to surgery, with a family history of thromboembolism, with an active cardiovascular disease, with chronic use of multiple medications, not responding as expected to drug therapy and with a previous ADR.

The DNA testing process can include using a DNA kit; collect a buccal swab in the mouth. The sample is labeled using a label automatically printed using the home care automated auditing system 2600 to automatically fill in the patient name and other pertinent information. The DNA test requisition form can also be automatically filled in using a physician's text or voice entered instructions, and both the sample and requisition form are sent to a laboratory that can be selected from a list of available DNA testing labs automatically maintained in the DNA testing and genetic disorders database 2612. The DNA sample test results will be automatically tracked in the DNA testing and genetic disorders database 2612 including an automatic inquiry to the lab on expected dates when the results will be available.

Pharmacogenetics (PGx) is a well-established science studying how an individual metabolizes medications. PGx testing provides individualized insight into complex treatment scenarios. Key genetic variations are known to be involved in the absorption, distribution and excretion of 30 classes of drugs. Genetic testing is frequently advised by the FDA and other international regulatory bodies.

The DNA test drug sensitivity and vascular risk test will help a physician personalize patient treatment, lowering the chance and severity of adverse reactions. Monitoring plasma drug levels can vary up to 1000-fold among patients who take a standard dose, even if they weigh the same. Common variants in an individual's genes cause each patient to respond uniquely to treatment, frequently resulting in adverse reactions or limited/no therapeutic benefit.

Shown in the description of automated prescription writing in FIG. 15B is an example of a wearable wrist device used for voice activated prescription writing. A doctor can use the wearable health care devices voice activated and recognition apparatus features of the wearable device to dictate a prescription. The prescription voice recognition dictation will automatically fill into a prescription form template. The wearable health care devices automatically check the patient's EMR for alerts on medication allergies and other potentially conflicting medications the patient is taking. The alerts provided by the application automated prescription writing feature can include an automatic query of the DNA testing and genetic disorders database 2612 alerts for possible DNA test drug sensitivity and vascular risk test indications using the personalized patient DNA test screening. Along with other prescription alerts will be DNA drug sensitivity alerts.

Drug-gene association guidelines can include alert messages that for example include information on drug sensitivity pharmacogenetic results including clinical consequences, cardiovascular/thrombosis risk management, pain medications, psychotropic medications and other medications. In addition the guidelines can include medication guidance including standard precautions, use with caution, consider alternatives, and dosing guidance.

For example a physician can make a medication first choice. The guidelines can indicate an increased sensitivity to medication first choice and suggest considering an alternative second drug or consider prescribing medication first choice at standard dose and monitoring the plasma concentrations of medication first choice respective resultant plasma types to guide medication first choice dose adjustments. In another case the medication first choice can show a DNA indicated possible non-response to medication first choice. Based on the genotype result, this patient may be a medication first choice rapid metabolizer, although the result is not definitive.

The guidelines can show the following recommendations that apply for medication first choice rapid metabolizers; consider alternative second drug or increase medication first choice dose by 100%. Another course of treatment can be adjusting a maintenance medication first choice dose according to plasma concentrations. The physician can choose from a recommended group of available alternative second drugs not sensitive to a rapid metabolizer function.

Genetic testing may reduce hospitalization due to complications by as much as 30% and the home care automated auditing system 2600 and DNA testing and genetic disorders database 2612 information can provide a health care providers with a means to integrate this useful personalized prescribing feature to aid in appropriate patient treatments that are safer and offer greater benefits to a patient condition while increasing the prevention of possible adverse drug reactions of one embodiment.

The advances in DNA testing and use in diagnostics have made available to the home care aspect of health care the ability to integrate those advances into the home care assessment of patient care. The ability to readily perform DNA testing in the home enables home care to provide biologics into the assessment of a patient's condition. A visiting nurse can perform a DNA test as ordered by the attending physician in the home concurrent with or separate from a routinely schedule home care visit.

DNA testing allows physicians to check a patient's genetic code to determine the probably that symptoms may be connected to a genetic disorder. The DNA results can also be used to assess the use of small molecule drugs or drugs developed under a target-based drug discovery program for specific genetic disorders.

The actual DNA test and procedures will be determined by the particular testing kit used. The physician will have selected from the DNA testing and genetic disorders database 2612 the available DNA testing kit to be used. The DNA testing and genetic disorders database 2612 will automatically check the inventory at the doctor's office to advise the physician and office personnel of available for that test kit. The DNA testing and genetic disorders database 2612 will automatically add a replacement to the next order for kits and other supplies. The nurse can pick up the DNA testing kit selected from the doctor's office prior to the visit.

The home care visiting portable electronic apparatus 2640 will record the visiting nurse trip and pickup to the office and scan the barcode of the testing kit. Upon arrival at the patient's location the visiting nurse will conduct a patient interview on family histories. A visiting nurse using the home care visiting portable electronic apparatus 2640 will be able to record the patient's comments on family histories that may indicate disorders of a genetic nature. The nurse will advise the patient on the procedure used for the DNA testing to allay any apprehension. The nurse will have the patient sign an authorization for the test. The nurse will consult with the procedure downloaded from the DNA testing and genetic disorders database 2612. As the nurse proceeds with the procedure they will select the steps completed in sequence. These steps will be recorded on the home care visiting portable electronic apparatus 2640. The steps will include for example the securing of the sample in a suitable container to prevent contamination. Upon complete of the testing procedures the nurse will secure the sample and with the label attached that has automatically been printed out and given to the nurse when picking up the kit. The departure of the nurse and subsequent drop-off of the sample to the doctor's office or if directed to the laboratory selected by the physician.

Upon receipt of the test results, the results will be scanned and entered into the DNA testing and genetic disorders database 2612. The DNA testing and genetic disorders database 2612 will compare the results with a DNA testing and genetic disorders database 2612 DNA library of genetic disorders to assess the potential of a match and send the comparative search results to the physician via email or text messaging. The physician will assess the biologic and DNA testing results to determine whether the patient has a genetically linked disorder, be receptive to small molecule drugs or target-based drugs available.

The DNA testing and genetic disorders database 2612 will maintain a database library on small molecule drugs or target-based drugs indicating for example side effects and treatment administration and other treatments available for genetic disorders. The physician will be able to review the results of an automated inquiry on drugs and treatments matching any indicated genetic disorders. If so indicated the physician will prescribe treatment accordingly. The automated home care database 2630 will also maintain information on genetic disorders to make available information the physician may select to be presented to the patient to educate the patient on the disorder and treatments available.

Home Care Visit DME Auditing:

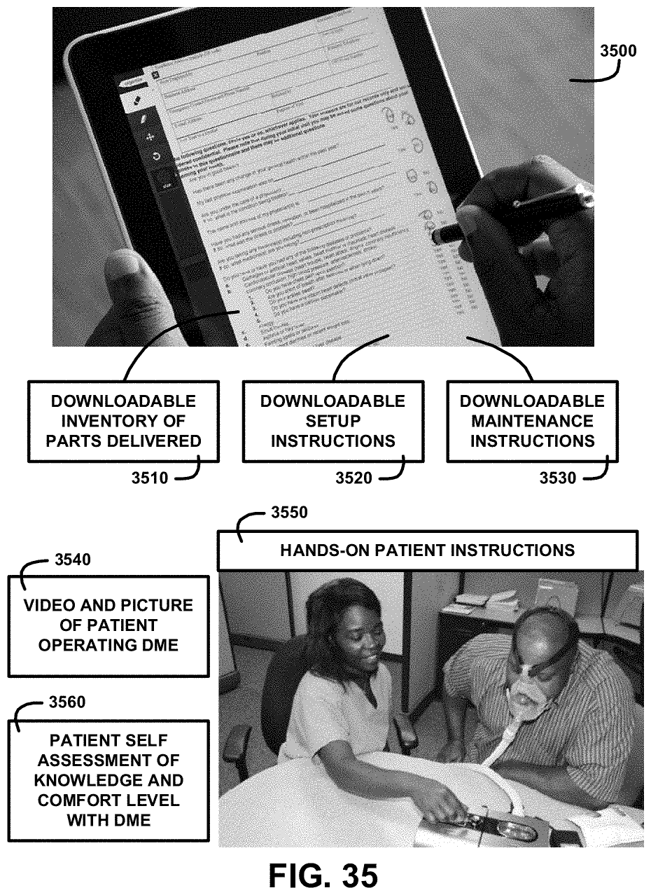

FIG. 35 shows for illustrative purposes only an example of home care visit DME auditing of one embodiment. FIG. 35 shows a DME setup application 3500. The DME setup application 3500 shows a downloadable inventory of parts delivered 3510, downloadable setup instructions 3520 and downloadable maintenance instructions 3530. The DME setup application 3500 allows a homecare provider to provide hands-on patient instructions 3550 on the use and maintenance of the DME. The DME setup application 3500 allows the homecare provider to capture video and picture of patient operating DME 3540. After the DME instructional sessions the patient takes a patient self-assessment of knowledge and comfort level with DME 3560 using the DME setup application 3500 of one embodiment.

Wearable Health Care Device Features:

FIG. 36 shows a block diagram of an overview of wearable health care device features of one embodiment. FIG. 36 shows wearable health care devices 3600 used for automated hands-free health care delivery. The wearable health care devices 3600 are embodiments of the device issuer locked electronic devices 490 of FIG. 4, the at least one portable electronic apparatus 2540 of FIG. 25 and the home care visiting portable electronic apparatus 2640 of FIG. 26. The wearable health care devices 3600 are configured to include at least one functionality algorithm embedded processor to analyze and calculate at least one home care automated auditing system functionality 2502 of FIG. 25. Wearable health care devices 3600 are used for interfacing with electronic and digital apparatus 3610 creating a hands-free tool to aid doctors and other health care professionals 3620. Wearable health care devices features 3602 include wireless reception and transmission 3630. The wearable health care devices provide a vehicle whereby doctors, health care professional including nurses and first responders, and non-health care individuals can use both hands for administering to a patient while automatically recording using voice, scanning, image capture and audio the patient's condition, the procedures being applied and interactions with others in remote circumstances over wireless connectivity.

Wearable health care devices features 3602 include image capture including digital photos, thermal imaging, ultraviolet (UV) highlighting, video 3640, audio capture including voice and sounds, including stethoscopic heart beats, respiration, and lung sounds during deep breathing 3650, digital direct and digital wireless recording including wireless digital thermometer readings, digital electrocardiogram (EKG) readings, digital electroencephalographic (EEG) readings and other medical and diagnostic digital equipment 3652, recording and retrieving electronic medical records (EMR) with patient information 3660, searching and processing insurance verifications 3662, and voice activated transcription for uses including filling out prescription, ordering x-rays and lab work, scheduling procedures, and other 3670 and other customized features. The wearable health care devices 3600 can be configured for a user to select a mode of accessing the wearable health care devices features 3602 including for example a physical button integrated into the wearable health care devices 3600 cases or container and/or touch screen activation areas and/or voice commands of one embodiment.

The wearable health care devices provide a vehicle whereby doctors, health care professional including nurses and first responders, and non-health care individuals can use both hands for administering to a patient while automatically recording using voice, scanning, image capture and audio the patient's condition, the procedures being applied and interactions with others in remote circumstances over wireless connectivity. The health care individual will automatically access the patient's electronic medical records (EMR) to check for alerts indicating allergies, other conditions that may influence the care being provided, personal contact information, other forms of treatment or medications the patient is taking. The database systems accessible using the wearable health care devices can be used to automatically searching and processing insurance verifications and applicable insurance treatment prices by using image capture to scan health insurance cards, patient drivers licenses and other identification documents, prescription labels and other documents of one embodiment.

The wearable health care devices include wireless reception and transmission features for interfacing with other electronic and digital apparatus. The wearable health care devices include a digital processor, a wireless connection including internet and a digital memory device. The wearable health care devices include features creating a hands-free tool to aid doctors and other health care professionals. The wearable health care devices include features for image capture including digital photos, thermal imaging, ultraviolet (UV) highlighting, video. Wearable health care devices features include audio capture including voice and sounds, including stethoscope heart beats, respiration, lung sounds during deep breathing of one embodiment.

Wearable health care devices features include digital direct and digital wireless recording including wireless digital thermometer readings, digital electrocardiogram (EKG) readings, digital electroencephalographic (EEG) readings and other medical and diagnostic digital equipment. Wearable health care devices features include recording and retrieving electronic medical records (EMR) with patient information. Features of wearable health care devices include voice activated transcription for uses including filling out prescription, ordering x-rays and lab work, scheduling procedures, and other uses. Wearable health care devices features include tele-medicine interfacing with health care personnel in remote locations including rural areas and searching Medicare acceptable tele-medicine locations of one embodiment.

Wearable Wrist Device:

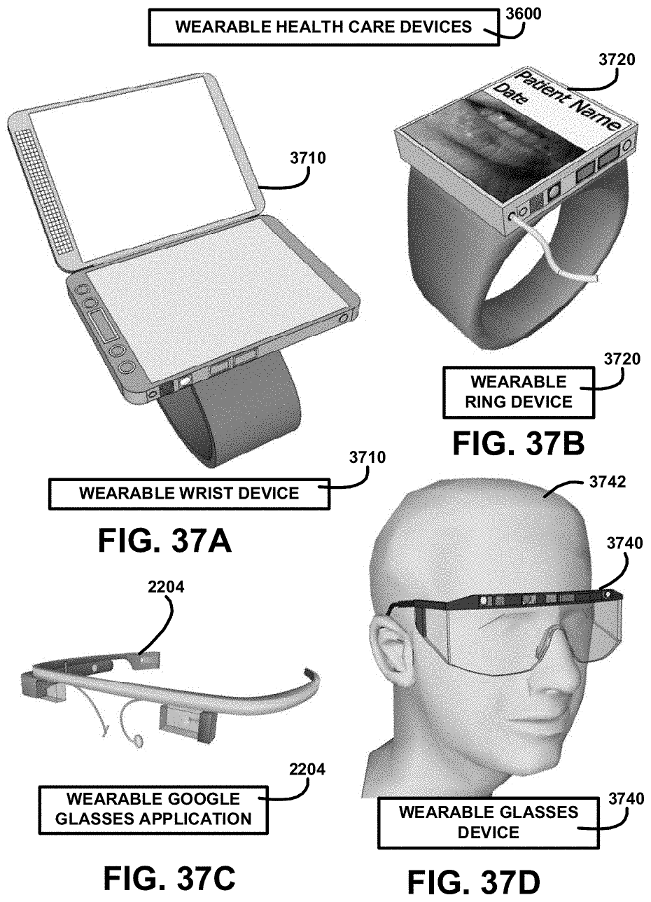

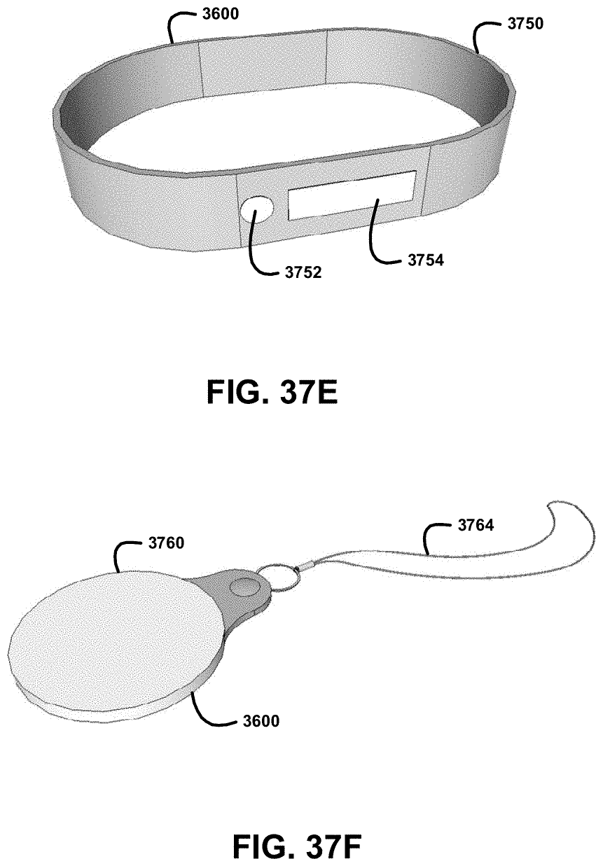

FIG. 37A shows for illustrative purposes only an example of a wearable wrist device of one embodiment. FIG. 37A shows one embodiment of wearable health care devices 3600. A wearable wrist device 3710 that can be worn by health care professionals for using the wearable health care devices features to provide hands-free interaction with a patient while performing tasks including for example automatically confirming patient identification, insurance verifications, checking other medications and patient conditions. The wearable wrist device can be used to perform diagnostic test and record the results of one embodiment.

Wearable Ring Device:

FIG. 37B shows for illustrative purposes only an example of a wearable ring device of one embodiment. FIG. 37B shows one embodiment of wearable health care devices 3600. A wearable ring device 3720 that can be worn by health care professionals for using the wearable health care devices 3600 features to provide hands-free interaction with a patient while performing tasks including for example automatically confirming patient identification, insurance verifications, checking other medications and patient conditions. The wearable wrist device 3720 can be used to perform diagnostic test and record the results of one embodiment.

Wearable Google Glasses Application:

FIG. 37C shows for illustrative purposes only an example of a google glasses application of one embodiment. FIG. 37C shows another embodiment of wearable health care devices 3600. A wearable google glasses application 2204 wherein the google glasses can be adapted to include additional wearable health care devices 3600 features and programmable application functionalities. The wearable google glasses application 2204 can be worn by health care professionals for using the wearable health care devices 3600 features to provide hands-free interaction with a patient while performing tasks including recording the results of a patient examination using a voice recording feature. Accessing a patient's EMR and connecting to other electronic and digital apparatus using wireless connectivity of one embodiment.

Wearable Glasses Device:

FIG. 37D shows for illustrative purposes only an example of a wearable glasses device of one embodiment. FIG. 37D shows one embodiment of wearable health care devices 3600. A wearable glasses device 3740 can be worn on a user head 3742 for example a health care professional. The wearable glasses device 3740 can be worn by health care professionals for using the wearable health care devices 3600 features to provide hands-free interaction with a patient while performing tasks including for example automatically confirming patient identification, insurance verifications, checking other medications and patient conditions. The wearable glasses device 3740 can be used to perform diagnostic test and record the results of one embodiment.

Wearable Wrist Band Device:

FIG. 37E shows for illustrative purposes only an example of a wearable wrist band device of one embodiment. FIG. 37E shows one embodiment of wearable health care devices 3600. A wearable wrist band device 3750 is configured to include a battery 3752 to power a digital electronic device 3754 configured to include a micro-processor to receive, record and process data, a wireless communication device configured to transmit the process data to the integrated mobile device management service provider digital programmable server 100 of FIG. 1, a temperature sensor used detect a patient's temperature, a sensor to detect a patient's heart beat (pulse), a GPS location device to detect a patient's physical location and a digital memory device to store data.

The wearable wrist band device 3750 is configured to be worn by a patient in recovery or in treatment in their living quarters. The device issuer of the wearable wrist band device 3750 can lock the functions of the wearable wrist band device 3750. The locked function can be configured to provide patient monitoring including location, the extent of movement, temperature and pulse while the patient is occupying their living quarters and moving about the area while recovering or under treatment.

For example a heart attack victim during recovery may be physically over stressing themselves wherein the locked functions of the wearable wrist band device 3750 communicated processed data show lengthy and frequent movement, periods of elevated temperature and pulse. The physician can communicate directly with the patient by for example telephone to advise the patient to reduce their activities. The physician can arrange a homecare provider visit wherein the homecare provider can advise the patient on the risks associated with the extensive activities and that the patient should immediately reduce the excessive activities. If the patient indicates they are having to shop for groceries or purchase meals from nearby restaurants the physician can make arrangements for assistance in meal deliveries to the patient at their living quarters to alleviate the cause of the over exertions of one embodiment.

Wearable Pendant Device:

FIG. 37F shows for illustrative purposes only an example of a wearable pendant device of one embodiment. FIG. 37F shows one embodiment of wearable health care devices 3600. A wearable pendant device 3760 is configured to include a neck cord 3764 to be used for wearing around a patient's neck. The wearable pendant device 3760 is configured to include a battery to power a digital electronic device configured to include a micro-processor to receive, record and process data, a wireless communication device configured to transmit the process data to the integrated mobile device management service provider digital programmable server 100 of FIG. 1, a temperature sensor used detect a patient's temperature, a sensor to detect a patient's heart beat (pulse), a GPS location device to detect a patient's physical location and a digital memory device to store data.

The wearable pendant device 3760 is configured to be worn by a patient in recovery or in treatment in their living quarters. The device issuer of the wearable pendant device 3760 can lock the functions of the wearable pendant device 3760. The locked function can be configured to provide patient monitoring including location, the extent of movement, temperature and pulse while the patient is occupying their living quarters and moving about the area while recovering or under treatment.

For example an orthopedic surgery patient during recovery may be physically over stressing for example their leg which underwent reconstructive surgery. The locked functions of the wearable pendant device 3760 communicated processed data show lengthy and frequent movement, periods of elevated temperature and pulse. The physician can communicate directly with the patient by for example telephone to advise the patient to reduce their activities. The physician can arrange a homecare provider visit wherein the homecare provider can advise the patient on the risks associated with the extensive activities and that the patient should immediately reduce the excessive activities. If the patient indicates they are having to get to a bathroom and a kitchen for food the physician can make arrangements for in-home assistance and DME mobility aids for the patient at their living quarters to alleviate the causes of the over exertions of one embodiment.

Wearable Glasses Device Features:

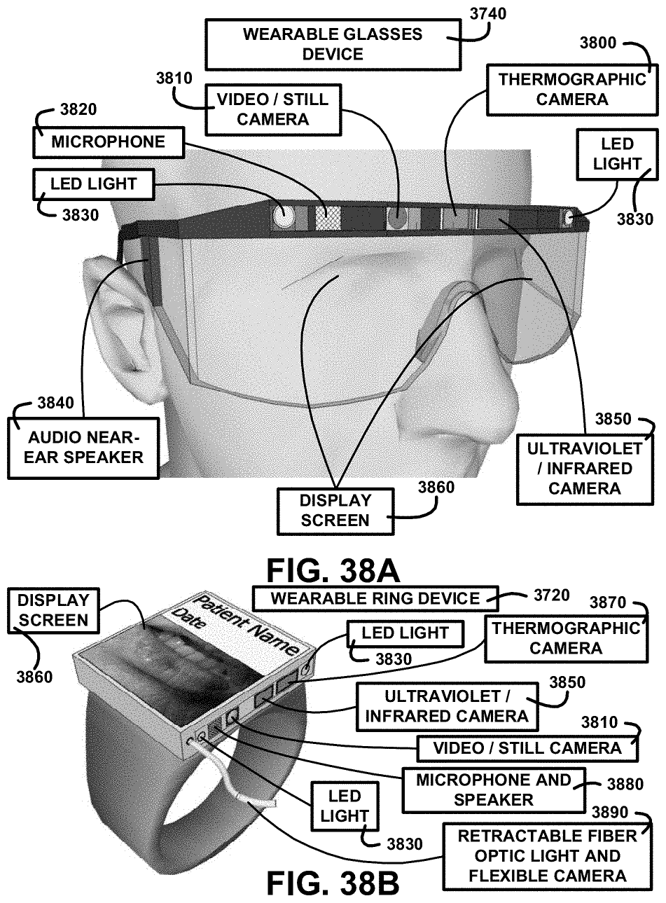

FIG. 38A shows for illustrative purposes only an example of a wearable ring device of one embodiment. FIG. 38A shows the wearable glasses device 3740 equipped with features including a video/still camera 3810, microphone 3820, one or more led light 3830, one or more audio near-ear speaker 3840, a thermographic camera 3800, an ultraviolet/infrared camera 3850 and one or more display screen 3860 of one embodiment.

A Wearable Ring Device Features:

FIG. 38B shows for illustrative purposes only an example of a wearable ring device features of one embodiment. FIG. 38B shows the wearable ring device 3720 equipped with wearable ring device features including a display screen 3860, one or more led light 3830, a thermographic camera 3800, an ultraviolet/infrared camera 3850, a video/still camera 3810, microphone and speaker 3880 and a retractable fiber optic light and flexible camera 3890 of one embodiment.

A Wearable Wrist Device Features:

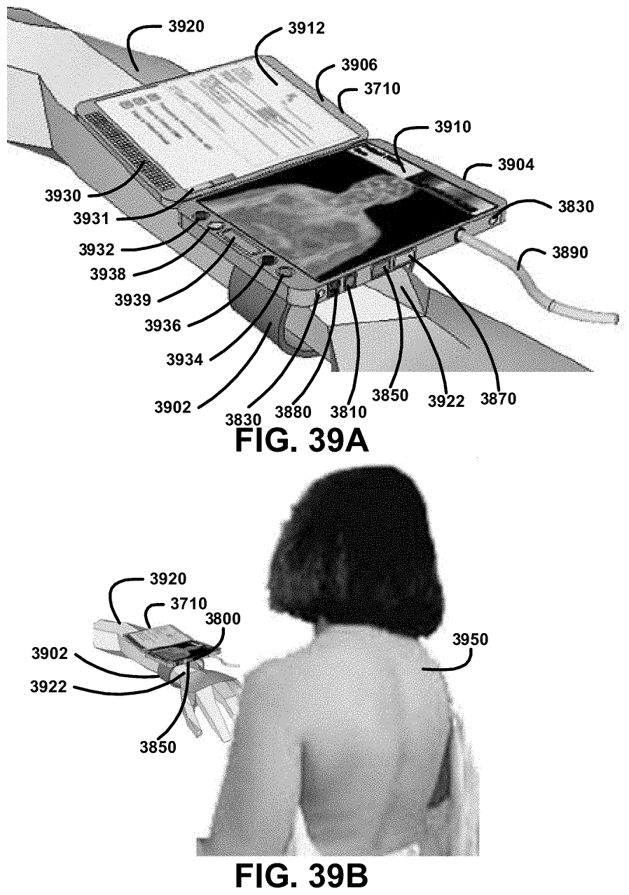

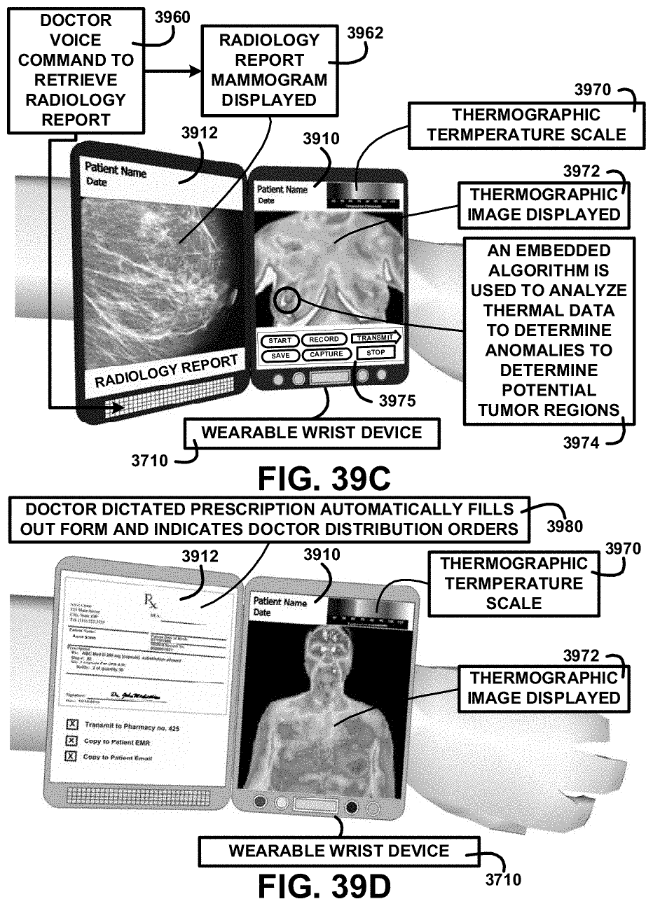

FIG. 39A shows for illustrative purposes only an example of a wearable wrist device features of one embodiment. FIG. 39A shows the wearable wrist device 3710 with features including a main case 3904 with a first display screen 3910 and a flip case 3906 with a second display screen 3912. The wearable wrist device 3710 features include an adjustable wrist band 3902. The adjustable wrist band 3902 feature is used to attach the wearable wrist device 3710 to either the right or left user forearm 3920 by wrapping the adjustable wrist band 3902 around a user wrist 3922 and adjusting the band for a firm fit. Additional wearable wrist device 3710 features include one or more led light 3830, a thermographic camera 3800, an ultraviolet/infrared camera 3850, a video/still camera 3810, a first microphone and speaker 3880. A retractable fiber optic light and flexible camera 3890 feature is included for use for example by a physician to capture video and still pictures of for example the inner ear, throat and/or interior of a nostril.

The wearable wrist device 3710 features include a first mode toggle button 3936 feature to select a feature operation. A feature operation includes for example the operation of the first display screen 3910, the second display screen 3912, one or more led light 3830, the thermographic camera 3800, the ultraviolet/infrared camera 3850, and/or the video/still camera 3810. Feature operations may be used simultaneously for example a user may select operation of the thermographic camera 3800 then press a start button 3934 to begin the operation wherein thermographic images are recorded. The user can then select another mode for example the video/still camera 3810. Again the user can press the start button 3934 to capture video images of the same view without thermographic readings to use as an external reference base. Both operations are being performed simultaneously.

A second mode toggle button 3938 feature is used to select variations in the operations of the features selected, for example the capture of a single image (frame) from the thermographic camera 3800 as it is recording video images. A capture button 3939 feature is used for capturing a single image and/or a data input. A stop button 3932 feature is used for stopping wearable health care devices 3600 of FIG. 36 features operations.

Other variations include adjusting brightness of the first and second display screens, displaying touch screen operations areas for example a brightness adjuster 3931, downloading data from and uploading data to the integrated mobile device management service provider digital programmable server 100 of FIG. 1. Shown on the flip case 3906 is another wearable wrist device 3710 feature, a dictation and form filling voice activated microphone 3930 of one embodiment.

A Wearable Wrist Device Used in an Examination:

FIG. 39B shows for illustrative purposes only an example of a wearable wrist device used in an examination of one embodiment. FIG. 39B shows the wearable wrist device 3710 attached to the user forearm 3920 at the user wrist 3922 with the adjustable wrist band 3902. The ultraviolet/infrared camera 3850 and the thermographic camera 3800 are in operation simultaneously to collect data from a female patient undergoing a breast examination 3950 of one embodiment.

A Wearable Wrist Device Displaying Examination Results:

FIG. 39C shows for illustrative purposes only an example of a wearable wrist device displaying examination results of one embodiment. FIG. 39C shows the wearable wrist device 3710 displaying results of the breast examination shown in FIG. 39B. FIG. 39C shows on the second display screen 3912 a radiology report mammogram displayed 3962 after a doctor voice command to retrieve radiology report 3960 from the integrated mobile device management service provider digital programmable server 100 of FIG. 1.

Shown on the first display screen 3910 is a thermographic temperature scale 3970 for use in a visual interpretation of a thermographic image displayed 3972. Also shown are touch screen operations areas 3975. The doctor has uploaded the examination results to the integrated mobile device management service provider digital programmable server 100 of FIG. 1 wherein an embedded algorithm is used to analyze thermal data to determine anomalies to determine potential tumor regions 3974. The integrated mobile device management service provider digital programmable server 100 of FIG. 1 has automatically downloaded the results of the algorithm analysis determination of anomalies and displays in the circle potential tumor regions of one embodiment.

A Wearable Wrist Device Used for Writing a Prescription:

FIG. 39D shows for illustrative purposes only an example of a wearable wrist device used for writing a prescription of one embodiment. FIG. 39D shows the wearable wrist device 3710 second display screen 3912 where a doctor dictated prescription automatically fills out form and indicates doctor distribution orders 3980 including orders to transmit to pharmacy no. 425, copy to patient EMR and copy to patient email. The first display screen 3910 is displaying the thermographic temperature scale 3970 and thermographic image displayed 3972. Pharmacies and prescription medications including generics can be displayed in integrated advertisement ad placement areas on the device issuer locked electronic devices 490 of FIG. 4 including the home care visiting portable electronic apparatus 2640 of FIG. 26, and wearable health care devices 3600 of FIG. 36 of one embodiment.

A Tele-Medicine Mobile Application:

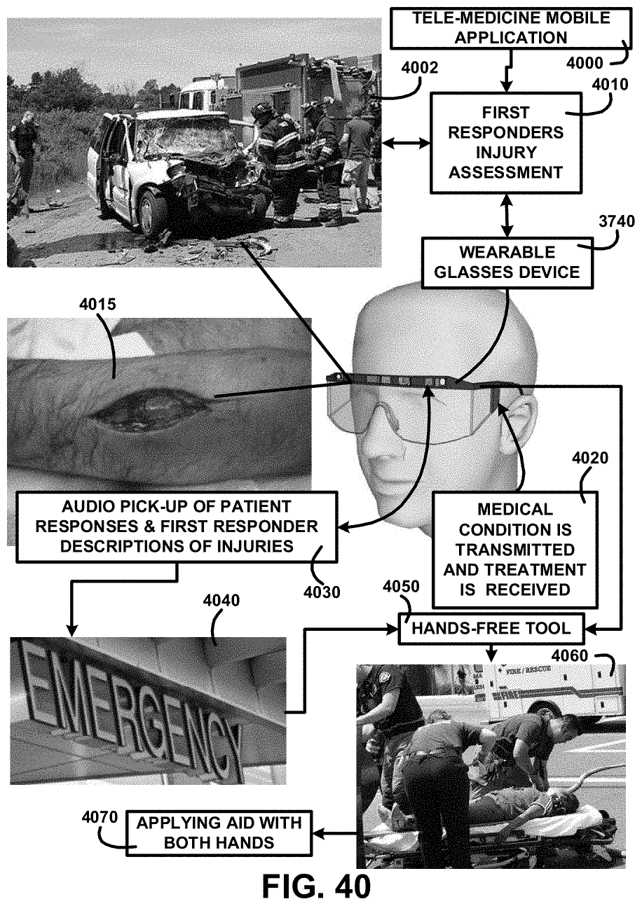

FIG. 40 shows for illustrative purposes only an example of a tele-medicine mobile application of one embodiment. FIG. 40 shows a tele-medicine mobile application 4000 using the wearable glasses device 3740. The wearable glasses device 3740 is used for a camera image capture of accident scene 4002 and for a function of a first responder's injury assessment 4010. The first responder's injury assessment 4010 includes a camera image capture of accident victim visible injury 4015 and an audio pick-up of patient responses & first responder descriptions of injuries 4030. The wearable glasses device 3740 is used for first responder communication with a remote emergency care unit, wherein a medical condition is transmitted and treatment is received 4020 from the remote emergency care unit. The wearable glasses device 3740 provides the first responder with a hands-free tool 4050 while performing aid. A remote emergency care unit in communication with first responders 4040 is receiving real-time information and data from first responders using both hands while capturing images and recording and transmitting medical conditions and treatments 4060. First responders are able to be applying aid with both hands 4070 and not having to stop and communicate the victim's condition thereby saving valuable time for direct aid of one embodiment.

Tele-Medicine Mobile Application for Use in Remote and Rural Areas:

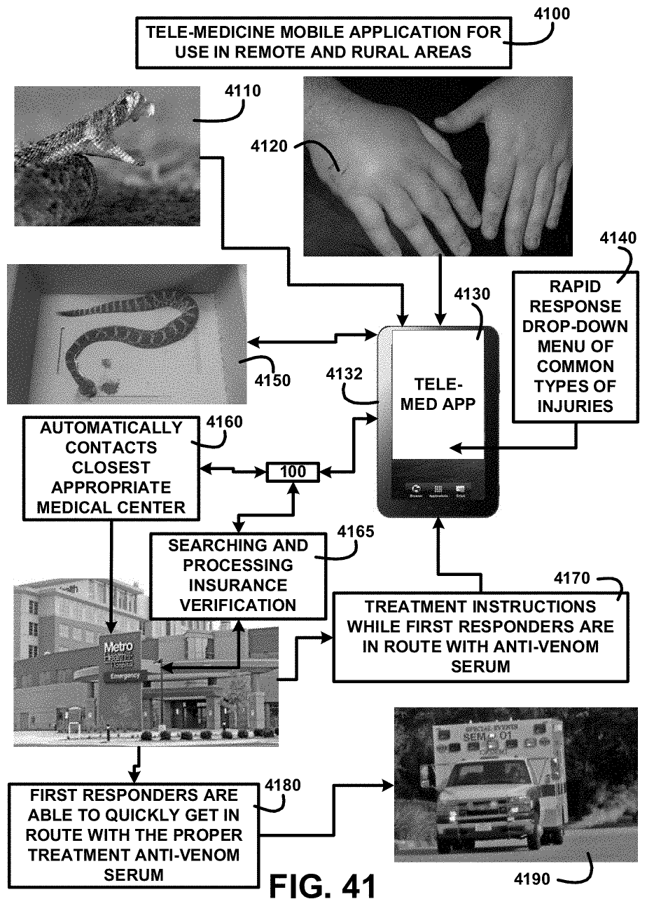

FIG. 41 shows for illustrative purposes only an example of tele-medicine mobile application for use in remote and rural areas of one embodiment. FIG. 41 shows a tele-medicine mobile application for use in remote and rural areas 4100, also referred to as a tele-med app 4130 herein, being used for example to respond to a snake bite incident. The tele-med app 4130 has been downloaded into a user portable digital communication device 4132. In this example a non-medically trained individual is using the tele-medicine mobile application for use in remote and rural areas 4100 to assist a companion or in an alternative themselves.

A camera image capture of snake before capture 4110 is taken with the portable digital communication device 4132 for example a smart phone. An additional camera image of snake after capture 4150 is taken. The two images of the snake are transmitted to the integrated mobile device management service provider digital programmable server 100 of FIG. 1 where a database of snake identification is used to aid in the identification of the snake species. The snake identification is queried through the database and sent to the user smart phone.

A rapid response drop-down menu of common types of injuries 4140 is displayed from which the user can select a snake bite. The integrated mobile device management service provider digital programmable server 100 of FIG. 1 is automatically queried and appropriate first-aid information is relayed back to the user smart phone for immediate use in treating the injury. Simultaneously upon receiving the rapid response selection of a snake bite injury the integrated mobile device management service provider digital programmable server 100 of FIG. 1 automatically using GPS locator of incident site and near-by medical centers contacts closest appropriate medical center 4160 and establishes communication with the user smart phone. An appropriate medical center may be one in this instance with specific anti-venom available for the identified snake.

The GPS incident location of the smart phone is relayed to the medical center and the notification of the snake bite injury and snake species identification. The user can communicate directly with the medical center to transmit a camera image capture of victim's snake bite wound 4120 using the communication link established by the integrated mobile device management service provider digital programmable server 100 of FIG. 1. The images of the snake are also transmitted and automatic search features uses images of the snake to search for match of the snake characteristics to aid in identifying the species of snake and potential anti-venom for treatment.

The medical center can dispatch a first responder vehicle to the victim's location using the GPS coordinates previous sent. First responders are able to quickly get in route with the proper treatment anti-venom serum 4180. The first aid information transmitted from the integrated mobile device management service provider digital programmable server 100 of FIG. 1 is used for treatment instructions while first responders are in route with anti-venom serum 4170 for example removing any rings or constricting items, because the affected area may swell.

The integrated mobile device management service provider digital programmable server 100 of FIG. 1 simultaneously is searching and processing insurance verification 4165 of the victim whose identification has been inputted using the user smart phone. The insurance verification results are automatically transmitted to the user smart phone and medical center. Upon arrival appropriate first responder aid is applied and first responders are transporting the victim to closest appropriate medical center 4190. The tele-medicine mobile application for use in remote and rural areas 4100 has provided the victim with rapid first aid instruction, automatically contact medical aid, and provider the health care professional with real-time information to allow them to make quick accurate assessments of the aid necessary and respond rapid to aid the victim of one embodiment.

A Wearable Glasses Device Used in Surgery:

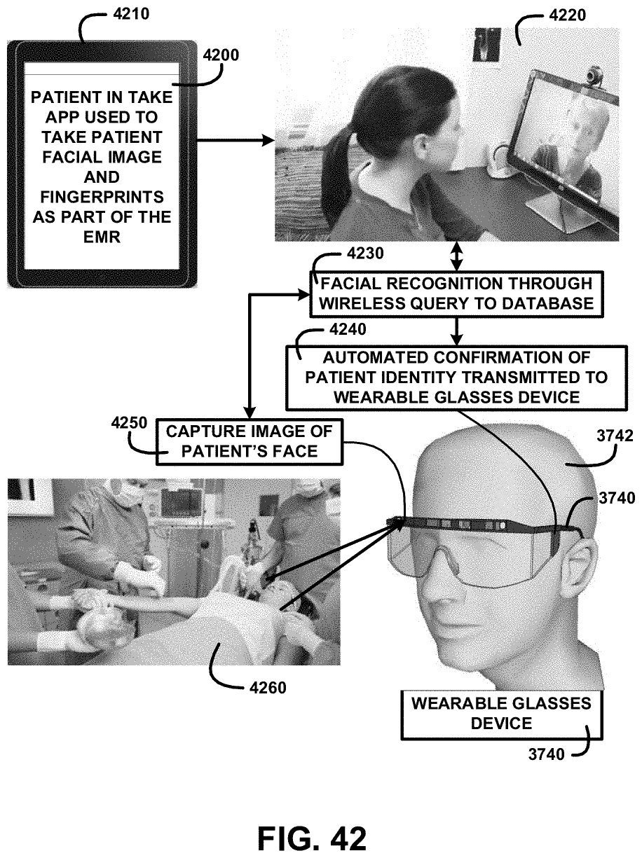

FIG. 42 shows for illustrative purposes only an example of a wearable glasses device used in surgery of one embodiment. FIG. 42 shows the wearable glasses device 3740 for use in surgery. The wearable glasses device 3740 is wirelessly connected to the integrated mobile device management service provider digital programmable server 100 of FIG. 1. The server database includes a patient in take app used to take patient facial image and fingerprints as part of the EMR 4200 captured using an electronic tablet with patient in take app downloaded 4210 at the time the patient was admitted to the hospital or from the doctor's office sign-in. The wearable glasses device 3740 is used to capture image of patient's face 4250. Simultaneously medical staff confirms whether patient is in their system using a facial recognition feature 4220. An image of patient's face is queried through the face recognition feature to confirm this is the correct person to undergo this specific surgery 4260.

An automatic functionality is used for facial recognition through wireless query to database 4230. An automated confirmation of patient identity transmitted to wearable glasses device 4240 assures the medical staff the correct patient is in the operating room. The wearable glasses device 3740 is worn on the user head 3742 to allow hands-free use of one embodiment.

An Automated Tablet and Wearable Device Connectivity:

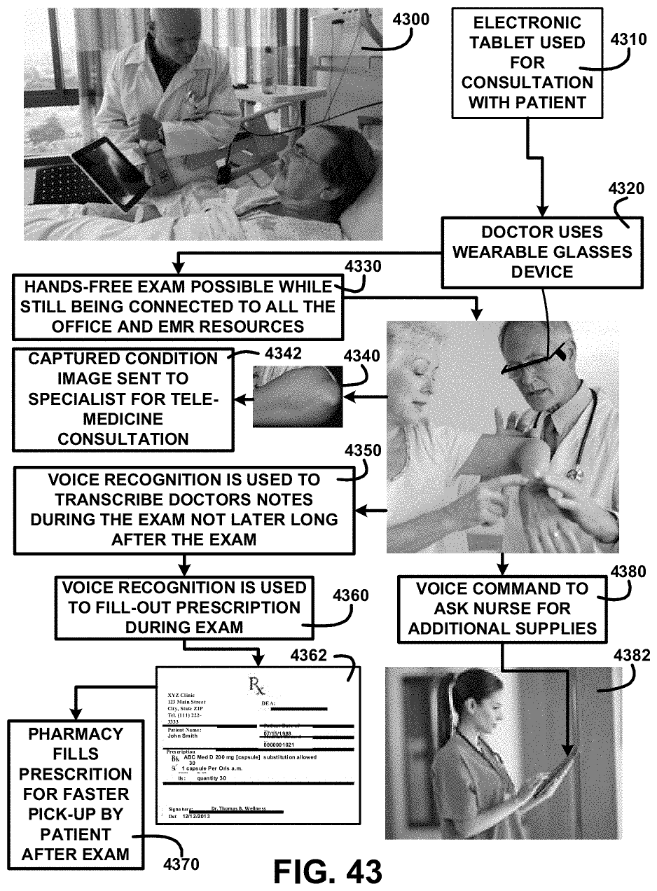

FIG. 43 shows for illustrative purposes only an example of an automated tablet and wearable device connectivity of one embodiment. FIG. 43 shows a multiple device issuer locked devices connectivity function including an automated tablet and wearable device connectivity to provide a network connection to assist health care providers with quick and complete access to assistance and information. The database can be accessed through a portable electronic apparatus including an electronic tablet used for initial consult with patient. A physician is consulting with a patient and explaining the patient condition using system images on an electronic tablet device 4300. An electronic tablet used for consultation with patient 4310 can also be used to record the consultation with patient including patient questions and concerns. The tablet coordinated care digital electronic platform can be used where a physician is consulting with a patient and explaining the patient condition using system images on a tablet device 4300. The tablet coordinated care digital electronic platform can be used for a tablet used for consultation with patient 4310 can also be used to record the consultation with patient including patient questions and concerns. The database can be accessed through a portable electronic apparatus including a tablet used for initial consult with patient 4300 including the tablet coordinated care digital electronic platform.

The portable electronic apparatus can for example be a wearable glasses device of one embodiment. The doctor uses a wearable glasses device 4320 while examining the patient. In this example the wearable glasses device can be used to verbalize a voice command to ask nurse for additional supplies 4380. The wearable glasses device transmits the voice command via a wireless connection to a tablet used by the nurse. The application can include an audio and text message to convey the voice command to the nurse immediately. An automated tablet is used by a nurse receiving request for additional supplies 4382 of one embodiment.

In another embodiment the doctor can use the wearable glasses device for hands-free exam possible while still being connected to all the office and EMR resources 4330. In this example the doctor uses voice commands to find any information related to a skin condition he sees on the patient's arm. The database returns the name and contact information of a specialist the patient had seen related to a similar condition a few years earlier. The doctor uses the wearable glasses device to capture an image of the patient's condition 4340 for example current skin condition.

A voice command is used to connect to the specialist. The device automatically sends the captured image to the specialist. The captured condition image sent to specialist for tele-medicine consultation 4342 allows the doctor, specialist and patient to discuss the current condition in real-time and agree that additional test are not needed and the patient does not need to report to a lab or ER for treatment or diagnostic purposes. The specialist transmits to the doctor's wearable glasses device the medication used previously to treat the condition. The same feature voice recognition is used to fill-out prescription during exam 4360. An electronic prescription form is filled out, transmitted and printed simultaneously 4362. The wearable glasses device following the doctor's voice commands distributes the prescription so that the pharmacy fills prescription for faster pick-up by patient after exam 4370 of one embodiment.

The doctor uses the device feature wherein voice recognition is used to dictate doctor's exam notes during the exam in real-time not later long after the exam. The voice recognition is used to transcribe doctor's notes during the exam not later long after the exam 4350.

During the examination period the doctor is fully connected to the resources he needs to thoroughly perform the exam, consult with other, research the patient's EMR and get assistance from other staff members. The patient is better serve by in this example preventing an additional trip to a lab or ER; the doctor is made aware of the previous occurrences of the condition and how to remedy the condition without any delay. All parties save time, money and collectively medical and health care resources which in addition to being expeditious will also lead to reducing the cost of health care of one embodiment.

Annual Wellness Visit (AWV) Database:

The home care automated auditing system using the universal automated patient intake system establishes the patient electronic medical records (EMR). The universal automated patient intake system collects an individual's medical/family history, a list of current providers and suppliers that are regularly involved in providing medical care to the individual, and Measurement of an individual's height, weight, BMI (or waist circumference, if appropriate), BP, and other routine measurements. In addition the patient's past medical records from previous medical providers, hospitalization and treatments including prescription are gathered automatically with authorization from the patient. The patient's current health insurance information is gathered using the universal automated patient intake system input from the patient's health insurance card and other information provided. The system using the Health Insurance Coverage Predetermination Database contacts the health insurance provider and enters into the EMR the health plan, coverage, co-payment schedules and other coding and detailed information used for billing purposes including pre-authorizations for services and treatments.

The pre-authorization, billing and collection preparation can be performed using the tablet coordinated care digital electronic platform to create letters of medical necessity, gather history and physicals, make copies of clinical notes and any other reasonable documentation from physicians required to satisfy claim payments. The tablet coordinated care digital electronic platform can be used for reporting and review by for example a physician to obtain electronic access to its requisition forms, letters of medical necessity, etc. for review and verification to ensure that all appropriate information has been received, entered and submitted for processing.

The patient's EMR information is accessible in addition to offices computers using a portable electronic apparatus including an electronic tablet, iPad, iPod, hands-free wearable health care devices or other digitally programmable device on which the physician has downloaded the application available from a subscription to the home care automated auditing system.

The home care automated auditing system includes the Automated home care database, Medical condition reference materials database, Home care medical procedures database, Durable medical equipment reference database, Patient self-assessment testing database and other databases including an Annual Wellness Visit (AWV) database including a Personalized Prevention Plan Services (PPPS) database, Transitional Care Management services (TCM) database and Complex Chronic Care Coordination (CCCC) database.

The Annual Wellness Visit (AWV) database draws information from the patient EMR to gather information used in the preparation of the Personalized Prevention Plan Services (PPPS), Transitional Care Management services (TCM) and Complex Chronic Care Coordination (CCCC). In addition to the information already in the EMR the home care automated auditing system is used to gather additional information and record and store the specific data used in the development of the Annual Wellness Visit services and plans.

Personalized Prevention Plan Services (PPPS) Database:

Information specific to the development of the Annual Wellness Visit services and plans can be entered for example in the Personalized Prevention Plan Services (PPPS) database by the authorized providers including for example physicians, non-physician practitioners, and providers submitting claims to Medicare contractors (carriers, Medicare Administrative Contractors (MACs), and/or Fiscal Intermediaries (FIs) for services provided to Medicare beneficiaries. The authorized providers can enter and retrieve data using the portable electronic apparatus including an electronic tablet, iPad, iPod, and hands-free wearable health care devices and other digitally programmable device on which the home care automated auditing system subscription application has been downloaded.

The Personalized Prevention Plan Services (PPPS) specific information can include detection of any cognitive impairment that the individual may have. Another set of specific information can include a Review of the individual's potential (risk factors) for depression, including current or past experiences with depression or other mood disorders, based on the use of an appropriate screening instrument for persons without a current diagnosis of depression, which the health professional may select from various available standardized screening tests designed for this purpose and recognized by national medical professional organizations. The standardized screening tests can be downloaded in the Patient self-assessment testing database. From the Patient self-assessment testing database the provider can administer the test to the patient using the portable electronic apparatus for example in a home care visit. If appropriate the test can be administered by a regularly visiting nurse in order to not create external anxiety in the patient. In addition the provider can access previous tests taken with the patient and stored in the Patient self-assessment testing database to be able to review a long term pattern of behavior and responses.

Similarly the provider can enter a Review of the individual's functional ability and level of safety based on direct observation, or the use of appropriate screening questions or a screening questionnaire, which the health professional may select from various available screening questions or standardized questionnaires designed for this purpose and recognized by national professional medical organizations. The screening questions or standardized questionnaires can be downloaded into the Patient self-assessment testing database and administered in office or during a home care visit using the portable electronic apparatus including an electronic tablet, iPad, iPod, hands-free wearable health care devices or other digitally programmable device on which the home care automated auditing system subscription application has been downloaded.

Another specific set of information for the PPPS includes a written screening schedule for the individual, such as a checklist for the next 5 to 10 years, as appropriate, based on recommendations of the United States Preventive Services Task Force (USPSTF) and the Advisory Committee on Immunization Practices (ACIP), as well as the individual's health status, screening history, and age-appropriate preventive services covered by Medicare. Having access to the current information in the patient's EMR provides future appointments with the patient's doctor, surgery or hospital stay and other providers making the scheduling process that much easier. Since the EMR is a permanent and continually updated database the ability to schedule long range screening visits or appointments is more practical than a series of short term appointments that may get overlooked if there is a change in screening provider. The PPPS is intended to be a long range program to track and evaluate a patient's progress or lack of progress in a condition or overall health and the maintenance of a full comprehensive record of the patient's health care and conditions is essential to the success of the preventative services having a positive impact on the patient's wellbeing.

In another set of specific set of information under a PPPS is a list of risk factors and conditions for which primary, secondary, or tertiary interventions are recommended or are underway for the individual, including any mental health conditions or any such risk factors or conditions that have been identified through an IPPE, and a list of treatment options and their associated risks and benefits. The list of risk factors, interventions and treatment options must be kept up to be relevant to the patient's success in minimizing any risk factors from negatively changing their health. The home care automated auditing system provides the ideal tool for recording and long term storage of the lists and progress in dealing with the risk factors.

Access to the past and current list of risk factors, interventions and treatment options better enables the provider with evaluating the progress to date and developing a going forward plan to assist the patient in preventing the risk becoming a reality in the form of a full onset of the potentially harmful conditions. The provider can access, record and retriever data in this set of information in an office setting or in a home visit using the portable electronic apparatus including an electronic tablet, iPad, iPod, hands-free wearable health care devices or other digitally programmable device on which the home care automated auditing system subscription application has been downloaded including patient assessments and evaluation using video, audio and voice activated commands to check vitals and record in real-time the results.

Yet another set of set of information under a PPPS is the development and Furnishing of personalized health advice to the individual and a referral, as appropriate, to health education or preventive counseling services or programs aimed at reducing identified risk factors and improving self-management, or community-based lifestyle interventions to reduce health risks and promote self-management and wellness, including weight loss, physical activity, smoking cessation, fall prevention, and nutrition. The home care automated auditing system subscription application using the portable electronic apparatus including an electronic tablet, iPad, iPod, hands-free wearable health care devices or other digitally programmable device is ideal for developing a thorough personalized health advice presentation that for example can be shown to the patient on the screen of an electronic tablet with the audio and text narrative. For patient that have difficulties with English the home care automated auditing system subscription application include both text and voice translations that can assist the patient in understanding the presentation and importance of the personalized health advice in their native language. The same electronic tablet in the example can be used simultaneously to record a video of the patient watching the presentation and the conversations between the provider and patient for evaluation afterwards to gauge a perception of the effectiveness and reception by the patient in order to determine the frequency and alternative delivery method that will be better received and understood by the patient.

The home care automated auditing system subscription application can download the presentation to a patient digital device so that the patient can review the material in the presentation privately and as often as they may need to implement the suggestion in the personalized health advice. The provider can use the home care automated auditing system subscription application to schedule automated replaying of the presentation on a patient's device or sent via internet to a patient's smart phone or email to act as a refresher for the patient to take the personalized health advice seriously and reach the realization that improving self-management in reducing health risks is for their own good and that they are the central character in making it a success. The home care automated auditing system and subscription application can be expanded to include any other element(s) determined appropriate by the Secretary of Health and Human Services through the National Coverage Determination (NCD) process.

The provider can also use the home care automated auditing system and subscription application in the preparation of, reporting and billing of the Personalized Prevention Plan Services (PPPS) by also accessing updated current forms and report formats that can be filled-in automatically with the information from the patient's EMR to retrieve the elements of the reporting including an individual's medical/family history, list of current providers and suppliers that are regularly involved in providing medical care to the individual and patient vitals and measurement of an individual's height, weight, BMI (or waist circumference, if appropriate), BP, and other routine measurements as deemed appropriate, based on the beneficiary's medical/family history.

The provider can also access the updated current billing requirements including forms that can be automatically filled-in using with the information from the patient's EMR. The Health Insurance Coverage Predetermination Database will automatically check and confirm current pricing and reimbursable costs associated with the services and payable under the patient's health plan including Medicare. For example tracking and billing Chronic Care Management (CCM) non-face-to-face services including communication electronically and by phone with the patient and other treating health professionals for care coordination, medication management, and being accessible 24 hours a day to patients and any care providers including physicians or other clinical staff and managing the creation and revision of electronic care plans. The Health Insurance Coverage Predetermination Database will provide access to updated current forms and report formats that can be filled-in automatically with the information from the patient's EMR including for example a letter of medical necessity, requisition forms for laboratory testing, surgical procedures and other treatments dictated by the doctor's diagnosis and recommended treatment programs that have previously been predetermined as eligible under the patient's health insurance plan coverage. The home care automated auditing system and subscription application can also be used by the provider to file via internet and wireless transmittal of the reporting and billing automatically, and the system will enter automatically the billing information in the providers accounting records and automatically set up reminders on the receipt of payments for the services.

Transitional Care Management Services (TCM) Database:

The Annual Wellness Visit (AWV) database includes post-discharge transitional care management including Transitional Care Management services (TCM) database for the recording and storage of Transitional Care Management services (TCM) information. The Transitional Care Management services (TCM) database can include a set of codes for Transitional Care Management services (TCM). TCM services are for an established patient who's medical and/or psychosocial problems require moderate or high complexity medical decision making during transitions in care. TCM services address any needed coordination of care performed by multiple disciplines and community service agencies.

Transitional Care Management services are used for the transition in patient care for example various combinations including from a health care facilities to the patient's community setting. Examples include Inpatient Hospital to Home, Partial Hospital to Domiciliary, Observation Status to Rest Home and Skilled Nursing Facility to Assisted Living. Included in TCM is Nursing Facility Transitional Care Management services including a face-to-face visit within the specified time frames; Interactive contact with the patient or caregiver within two business days of discharge and may be direct (face-to-face), telephonic or electronic; and Medicare reconciliation and management no later than the date of the face-to-face visit.

The providers select from the TCM set of codes that include reported once per patient within 30 days of discharge; selected based on medical decision making and the date of the first face-to-face visit; and may be reported by one individual. Addition codes used to report these services include Transitional Care Management Services with the following required elements including communication (direct contact, telephone, electronic) with the patient and/or caregiver within two business days of discharge which may require good coordination with the hospital about the discharge date; medical decision making of at least moderate complexity during the service period; and face-to-face visit, within 14 calendar days of discharge.

Another group includes Transitional Care Management (TCM) Services with the following required elements include communication (direct contract, telephonic, electronic) with the patient and/or caregiver within two business days of discharge; medical decision making of high complexity during the service period; and face-to-face visit, within seven calendar days of discharge.

Additionally TCM codes include the following services including care plan oversight services; prolonged services without direct patient contact; anticoagulant management; medical team conferences; and education and training. Further the TCM codes include telephone services; end stage renal disease services; on-line medical evaluation; preparation of special reports; analysis of data; and medication therapy management. Physicians who provided hospital inpatient or observation services the billing can include typical times and allowed prolonged services to that set of observation or inpatient hospital services when the care (unit or floor time) exceeds the typical time. Billing for these services using the set of codes includes prices according to the Medicare Physician Fee Schedule (MPFS). The provider will also include collecting from the patient toward their deductible including Medicare Part B, Medicare Advantage Plans, Medicare secondary or supplemental insurance and other health insurance plans.

Preventive Services include for example the Medicare Initial Preventive Physical Examination (IPPE). Primary Care Incentive Program (PCIP) services including New and established patient office or other outpatient services; Nursing facility care visits and domiciliary, rest home or homecare plan oversight services; and Patient home visits. The provider for these services must be qualified with a designated for family medicine, geriatric medicine, pediatric medicine, internal medicine, nurse practitioner, clinical nurse specialist or physician assistant.

The home care automated auditing system and subscription application can be used by the provider to document and record the communication (direct contact, telephone, electronic) with the patient and/or caregiver; face-to-face visits; telephone services; end stage renal disease services; on-line medical evaluation; preparation of special reports; analysis of data; medication therapy management; and hospital inpatient or observation services.

The provider can easily record these services in an office or hospital setting or in a home visit using the portable electronic apparatus including an electronic tablet, iPad, iPod, hands-free wearable health care devices or other digitally programmable device on which the home care automated auditing system subscription application has been downloaded. For example observations can be recorded both in images and audio using the hands-free wearable health care devices while communicating face to face with a patient. The dialogue with the patient can automatically be transcribed using the voice to text features of the application. Patient education can be done for example using text to voice audio and streaming video on an electronic tablet.

In addition the provider can prepare, store and retrieve the post-discharge transitional care management using the Transitional Care Management services (TCM) database. The provider, having access to the patient's EMR, can easily include the patient conditions, personal information and medical history into the plan preparation, progress reporting and billing automatically filling in the data to the updated current forms from the database. The billing can automatically be prepared automatically and transmitted for payment using the Health Insurance Coverage Predetermination Database thereby saving billing time and delays in payments due to errors in the coding, allowable amounts, attachments of services reports and other details that are forgotten, or misapplied.

Complex Chronic Care Coordination (CCCC) Database:

The Annual Wellness Visit (AWV) database includes a Complex Chronic Care Coordination (CCCC) database. The Complex Chronic Care Coordination (CCCC) database is used for recording, storing and retrieving data specific to Complex Chronic Care Coordination (CCCC). CCCC services include patient centered management and support services provided by physicians, other qualified health professional and clinical staff; provided to an individual residing in a home or in a domiciliary, rest home or assisted living facility (no skilled nursing facility and no hospice); a care plan directed by a physician or qualified health professional and typically implemented by clinical staff; or services that address the coordination of care by multiple disciplines and community service agencies.

The provider provides or oversees the management and/or coordination of services, as needed, for all medical conditions; psychosocial needs; and activities of daily living. Coordination of care is an important factor to a patient's wellbeing. The provider does not have to actually provide the care for all of the above, but the provider must be coordinating that care. Elements of CCCC services and patients include patients requiring CCCC may be identified by algorithms that utilize reported conditions and services (e.g., predictive modeling risk score or repeat admissions or emergency department use); and clinical judgment.

CCCC patients typically have one or more chronic, continuous or episodic health conditions; commonly require the coordination of a number of specialties and services may have medical and psychiatric behavioral co-morbidities complicating their care; and may have social support weaknesses or difficulties accessing care. The CCCC services include codes used to describe the service and for billing including care plan oversight services; prolonged services without direct patient contact; anticoagulant management; medical team conferences; education and training; telephone services; end stage renal disease services; on-line medical evaluation; preparation of special reports; analysis of data; and medication therapy management.

The home care automated auditing system and subscription application can be used by the provider to document and record the communication (direct contact, telephone, electronic) with the patient, caregiver, physicians, other qualified health professional and clinical staff in the coordination of care by the multiple disciplines and community service agencies. The provider can access, record and retriever data from the Complex Chronic Care Coordination (CCCC) database in an office setting or in a home visit using the portable electronic apparatus including an electronic tablet, iPad, iPod, hands-free wearable health care devices or other digitally programmable device on which the home care automated auditing system subscription application has been downloaded for providing and overseeing the management and/or coordination of services including all medical conditions; psychosocial needs and activities of daily living. For example the provider can be visiting the patient to check on the activities of daily living and using the hands-free wearable glasses device record the patient going the paces of preparing a meal, taking a walk, or visiting a dialysis treatment center.

The portable electronic apparatus for example an electronic tablet can be used to tele-conference with the medical doctor and psychologist to discuss medications and patient behavioral and psychological response to a new prescription to determine its effectiveness. The tele-conference can be recorded and a transcription along with the video recording with audio stored in the database and used in reporting the patient condition under the management coordination billing.

The provider, having access to the patient's EMR, can easily include the patient conditions, personal information and medical history into the management coordination plan preparation, progress reporting and billing automatically filling in the data to the updated current forms from the database. CCCC billing can get complex to administer as the services codes including backup reports are reported once per calendar month; include all non-face-to-face CCCC services; include no or one face-to-face office or other outpatient, home or domiciliary visit; may only be reported by the single physician or qualified health care professional (QHP) who assumes the coordination role with a particular patient for the calendar month; and require determining the time spent.

The Complex Chronic Care Coordination (CCCC) database stored data including visits, conferences, patient observations, and checking on patient progress in wellbeing including the daily living activities can be retrieved and automatically fill the data into the backup reporting formats or forms. The CCCC database can also automatically track the time spent on the various management and coordination activities and used in the billing. The billing can automatically be prepared automatically and transmitted for payment using the Health Insurance Coverage Predetermination Database thereby saving billing time and delays in payments due to errors in the coding, allowable amounts, attachments of services reports and other details that are forgotten, or misapplied.

Patient Portal Application:

The home care automated auditing system includes a patient portal application. The patient portal application provides subscribing patients with for example full access to their EMR. The patient can for example look up health care bills, lab reports, doctor's reports on their condition and other information pertaining to their medical care. The patient can also using the patient portal application interact and communicate with their healthcare providers, such as physicians and hospitals. This feature allows the patient to for example make appointments, report on their current condition and let a doctor know the new medication does not agree with them and other interactive communications.

In one embodiment the patient will not have authorization to alter or change any medical reports include lab test, a doctors diagnosis and other sensitive data. The patient can still update their current personal information for example a change of address or phone number, their present condition and a new symptom. This feature of the patient portal application prevents unintentional changes and intentional changes and protects the integrity of the EMR. In one embodiment each health care providers is assigned an unlisted login account with which they can assign protection to data they enter into a patient's EMR to prevent alterations of a document or other form of data when they enter or download the data into the home care automated auditing system. Without the health care providers login information authorizing editing access to data protected by the unlisted login account designation the data cannot be edited or deleted. The embodiment of an additional security feature further enables compliance with HIPAA regulations of one embodiment.

In another embodiment the patient can through subscribing doctors, clinics, hospitals and other types of health care providers or organizations including health insurance carriers perform one universal login and through an internal cross referencing apparatus review their EMR in each of the subscribing health care location. This feature provides a broader access and prevents the patient from having to login to multiple health care locations individually. Multiple logins can be very inconvenient to older patience and may not give the patient an overall picture of their treatment, all lab reports showing different types of testing and other aspects of their care and cause confusion of one embodiment.

In another embodiment the patient can download portions of their EMR as they wish to show for example to a new doctor or results of outside lab tests that their existing doctors had not seen. The patient portal application provides the ability for the patient to print, fax, email, display, show a video or play an audio recording of the information they wish to share. Additionally the patient can use the patient portal application on a computer, laptop, a portable electronic apparatus including a smart phone, electronic tablet, iPad, iPod, hands-free wearable health care devices including wearable wrist device, wearable ring device, Google glasses, wearable glasses device, wearable wrist band device, wearable pendant device and other digitally programmable devices of one embodiment.

Medication Therapy Management:

The home care automated auditing system is an invaluable tool for health care providers to practice medication therapy management. The home care automated auditing system provides access to a complete patient EMR including all of the medication prescribed both past and present through a medication therapy management database and application. The medication therapy management database and application can include automated listings of all medication and their prescribed usage and dosage including allergies, conflicts between medication, symptoms and diagnostics associated with each medication and medical condition and other pertinent data determined from automated queries of the EMR data.

The medication listing can include patient reactions and progress to each medication. Reports and lab testing in the EMR can be cross-referenced to each medication. These reports and lab testing can include blood test showing progress and contradictory results for each medication. The reports and lab testing can include diagnostic data and indicators for example DNA test results and family history data to provide additional fine-tuning to a diagnosis to identify specific medication to treat the condition without conflicting with other medications.

The whole of the medication therapy management database and application provision of a complete medication record can prevent miss-prescribing and prescriptions that increase the effectiveness of medication therapy for patient treatment, controlling medication expense and pinpointed prescriptions for more accurately managing the entirety of a patient's medication. This feature creates a useful tool for providers for use in prescribing.

The medication therapy management database and application provides what may be the only method with which a provider can easily access a complete picture of a patient's medication that can involve multiple providers, clinics and hospitals without having to solely rely of what a patient can remember. Many times unintentionally incorrect prescription can be detrimental to a patient's treatment and unfortunately may lead to worsening the condition and even death. The ability to access and assess an accurate and complete prescription history provides the data and information to prevent incorrect prescribing saving both unnecessary expense and potential patient injury of one embodiment.

Preventative Health MACRA Compliance Digital System:

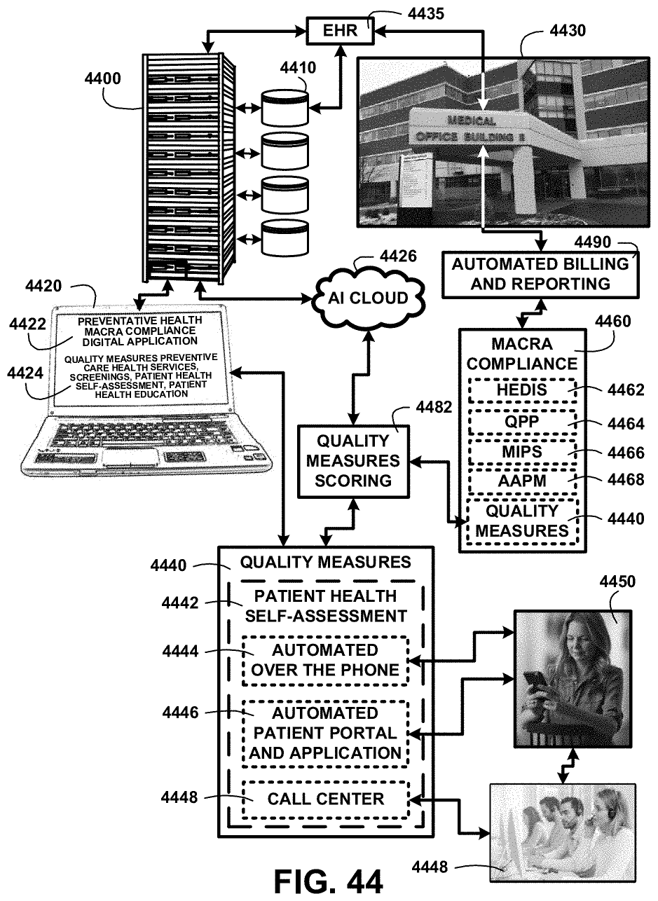

FIG. 44 shows for illustrative purposes only an example of an overview of a preventative health MACRA compliance digital system of one embodiment. FIG. 44 shows a preventative health MACRA compliance digital system including a network server 4400, plurality of databases 4410, AI cloud 4426, network server computer 4420 with a preventative health MACRA compliance digital application 4422. The preventative health MACRA compliance digital system is used for quality measures preventive care health services, screenings, patient health self-assessment, patient health education 4424.

Quality measures 4440 are used for compliance with the MACRA law compliance which focuses on preventative health care and establishes a scoring system to determine the efficacy of health care providers services to patients to prevent disease and health condition and sets up various payment systems and fee schedules to reward health care providers for preventing health issues rather than just treating health issues. Quality measures include patient health self-assessment 4442 measures that using the preventative health MACRA compliance digital system provides patients with automated over the phone 4444, automated patient portal and application 4446 and call center 4448 systems to remind, facilitate access and complete the self-assessments by the patients.

The patient completes the self-assessment in advance of a doctor office annual wellness visit. This shortens the patient 4450 visit to the medical office 4430 where vitals are taken including blood pressure and other data collection. The preventative health MACRA compliance digital system reduces doctor and medical staff time in meeting MACRA compliance 4460 and completing HEDIS 4462 quality measures. The preventative health MACRA compliance digital system automated systems further provides automated billing for the MACRA fee schedule billing in the three payment programs including QPP 4464, MIPS 4466 AND AAPM 4468. The preventative health MACRA compliance digital system assist both the patient and health care provider in performing the quality measures 4440, monitoring patient progress and analyzing quality measures scoring 4482 using the AI cloud 4426 and recording results in the patient EHR 4435, preparing compliance reports and assisting the doctor with evaluating the preventative measure results of one embodiment.

### Preventive Quality Measures:

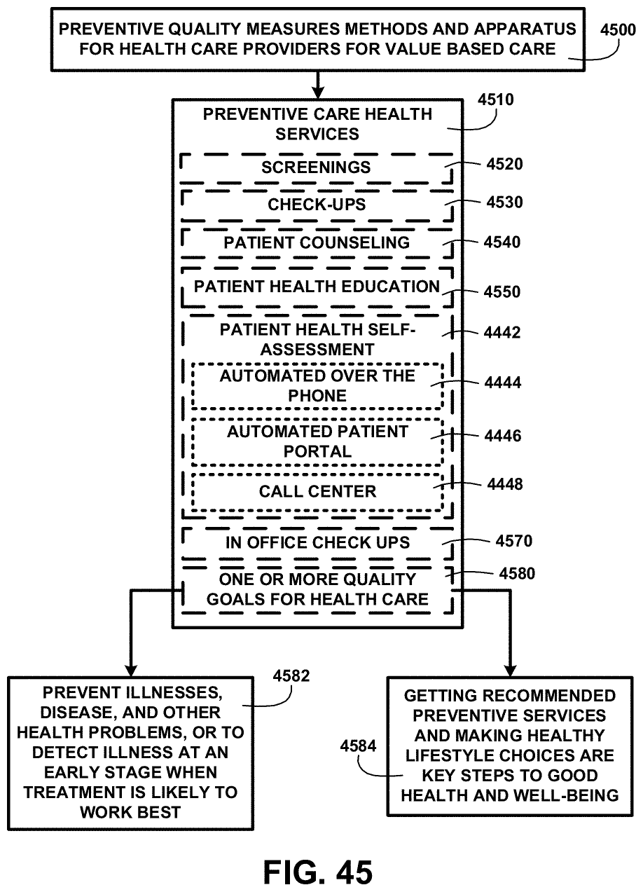

FIG. 45 shows for illustrative purposes only an example of preventive quality measures of one embodiment. FIG. 45 shows a system for preventive quality measures methods and apparatus for health care providers for value based care 4500. Quality measures include preventive care health services 4510 including screenings 4520, check-ups 4530, patient counseling 4540, patient health education 4550, patient health self-assessment 4442 which a patient may fill-out using an automated over the phone 4444, automated patient portal 4446, or call center 4448 system. The quality measures also include in office checkups 4570. One or more quality goals for health care 4580 are to prevent illnesses, disease, and other health problems, or to detect illness at an early stage when treatment is likely to work best 4582 of one embodiment.

### Call Center and Automated Over the Phone:

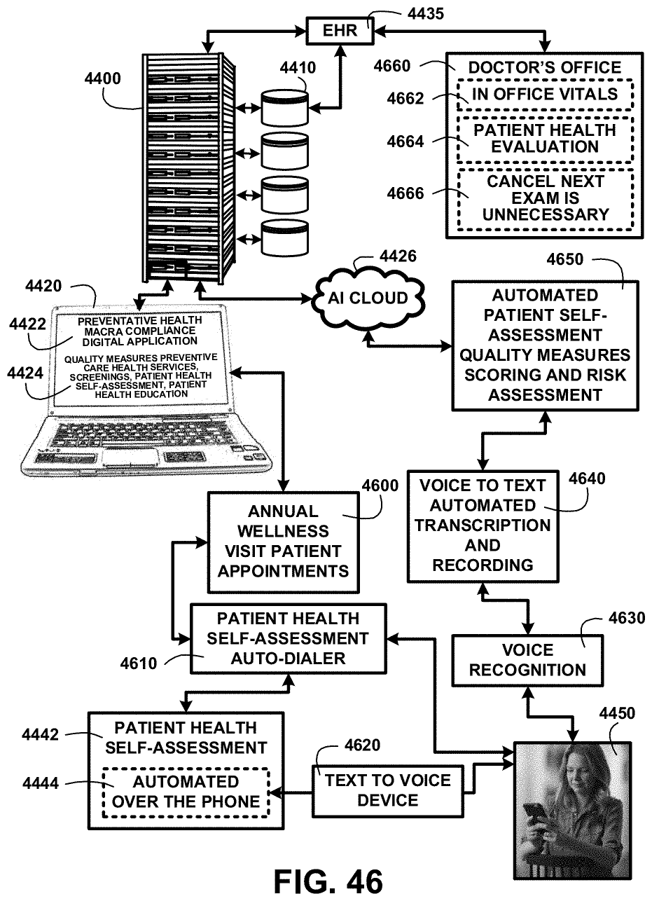

FIG. 46 shows for illustrative purposes only an example of a Call center and automated over the phone of one embodiment. FIG. 46 shows the network server 4400, plurality of databases 4410, AI cloud 4426 and network server computer 4420 with the preventative health MACRA compliance digital application 4422. The system is used for quality measures preventive care health services, screenings, patient health self-assessment, patient health education 4424. The system queries the annual wellness visit patient appointments 4600 and places a call in advance to the patient using a patient health self-assessment auto-dialer 4610. The patient may respond to the patient health self-assessment 4442 using the automated over the phone 4444 and may select the assessment be made in a verbal manner using a text to voice device 4620. When the patient 4450 speaks a response a voice recognition 4630 device uses voice to text automated transcription and recording 4640 to record the patient's responses. The responses are transmitted to the AI cloud 4426 for automated patient self-assessment quality measures scoring and risk assessment 4650. The AI cloud 4426 scoring and risk assessment are recorded in the patient EHR 4435 and also sent to the doctor's office 4660. At the AWV in office vitals 4662 are taken and recorded in the EHR 4435. The combined information is analyzed by the AI cloud 4426 for a patient health evaluation 4664 which is recorded and sent to the doctor's office. Based on the evaluations the doctor may find and cancel next exam is unnecessary 4666 of one embodiment.

Automated Patient Portal:

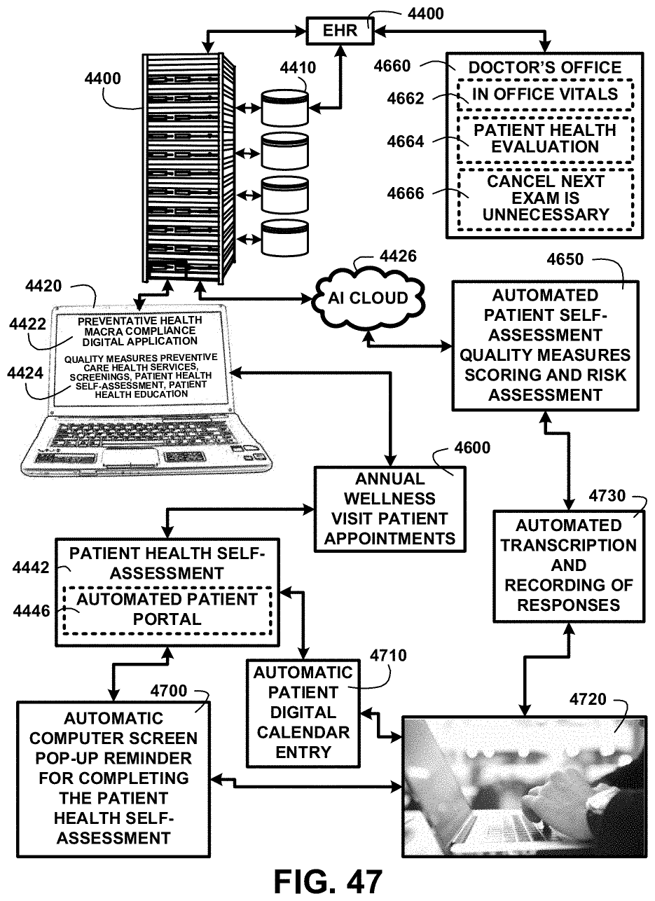

FIG. 47 shows for illustrative purposes only an example of automated patient portal of one embodiment. FIG. 47 shows a network server 4400 with a plurality of databases 4410, an artificial intelligence AI cloud 4426 and a network server computer 4420 with a preventative health MACRA compliance digital application 4422 installed. Quality measures preventive care health services, screenings, patient health self-assessment, patient health education 4424 are maintained in the plurality of databases 4410. The annual wellness visit patient appointments 4600 is tracked using the AI cloud 4426 and automatically sends in advance of the patient's appointment an automatic computer screen pop-up reminder for completing the patient health self-assessment 4700 through the automated patient portal 4446. Additionally in advance the system creates an automatic patient digital calendar entry 4710 for the appointment and prior patient reporting of the patient health self-assessment 4442 using the automated patient portal 4446.

A patient using a computer 4720 is shown filling out the patient health self-assessment 4442. When submitted the system creates an automated transcription and recording of responses 4730. The AI cloud 4426 performs and analysis for automated patient self-assessment quality measures scoring and risk assessment 4650. The network server 4400 records the AI cloud 4426 quality measures scoring and risk assessment in the patient EHR 4435 and sends a copy to the doctor's office 4660. Upon arrival to the AWV the medical staff takes in office vitals 4662 and enters those in the patient HER 4435. The AI cloud 4426 performs a patient health evaluation 4664 and sends the evaluation report to the doctor's office. Based on the evaluation the doctor may cancel next exam is unnecessary 4666 of one embodiment.

### Patient Personalized Prevention Plan Digital Application:

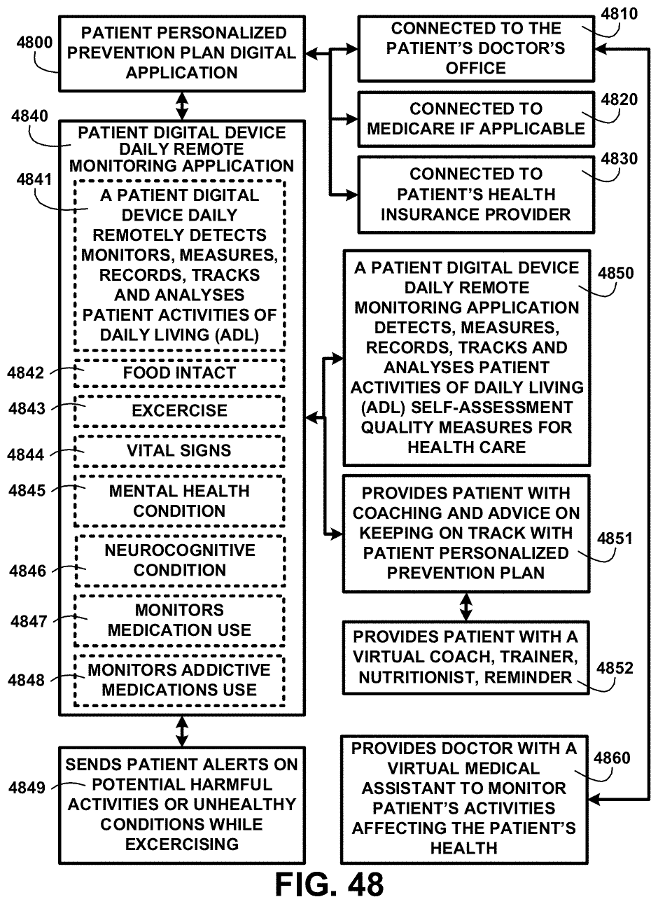

FIG. 48 shows for illustrative purposes only an example of patient personalized prevention plan digital application of one embodiment. FIG. 48 shows a patient personalized prevention plan digital application 4800. The patient personalized prevention plan digital application 4800 is a patient digital device daily remote monitoring application 4840. A patient digital device daily remotely detects monitors, measures, records, tracks and analyses patient activities of daily living (ADL) 4841 including food intact 4842, exercise 4843, vital signs 4844, mental health condition 4845, neurocognitive condition 4846, monitors medication use 4847, and monitors addictive medications use 4848.

Food intact 4842 includes a patient takes photo of food before eating. The artificial intelligence identifies foods and calculates calories, sodium and other elements. Exercise 4843 includes where the application counts steps and distance while walking, records time spent and calories utilized for the exercise. Vital signs 4844 are monitored using the application for periodically measures pulse, blood pressure, perspiration, breathing, balance and others.

Mental health condition 4845 where the application randomly asks the patient questions about how they are feeling, any anxiety, anger, fear, loneliness, and/or depression. Neurocognitive condition 4846 where the application has the patient perform cognitive functions to gauge their neurocognitive condition. Monitors medication use 4847 using the application the patient reports how often they are taking the medication. The application has the patient take a pic of the med container and analyses the contents for a number and volume of the medication to confirm the reported uses or any discrepancy.

Monitors addictive medications use 4848 using the application the patient reports how often they are taking the medication. The application has the patient take a pic of the med container and analyses the contents for a number and volume of the medication to confirm the reported uses or any discrepancy. The artificial intelligence identifies areas where a patient is lapsing off track and alerts where continued lapses may be detrimental to the patient's health and where changes in treatments, medications, and the personalized preventative plan needs adjustments.

The patient personalized prevention plan digital application 4800 sends patient alerts on potential harmful activities or unhealthy conditions while exercising 4849. The patient personalized prevention plan digital application 4800 is connected to the patient's doctor's office 4810, connected to Medicare if applicable 4820 and connected to patient's health insurance provider 4830. The patient personalized prevention plan digital application 4800 also sends alerts to the doctor's office, Medicare where applicable and the patient personalized prevention plan digital application 4800.

The patient personalized prevention plan digital application 4800 installed on a patient digital device daily remote monitoring application detects, measures, records, tracks and analyses patient activities of daily living (ADL) self-assessment quality measures for health care 4850. The patient personalized prevention plan digital application 4800 provides patient with coaching and advice on keeping on track with patient personalized prevention plan 4851, provides patient with a virtual coach, trainer, nutritionist, reminder 4852. The patient personalized prevention plan digital application 4800 provides doctor with a virtual medical assistant to monitor patient's activities affecting the patient's health 4860 of one embodiment.

Preventive Care for Adults Patient Health Education:

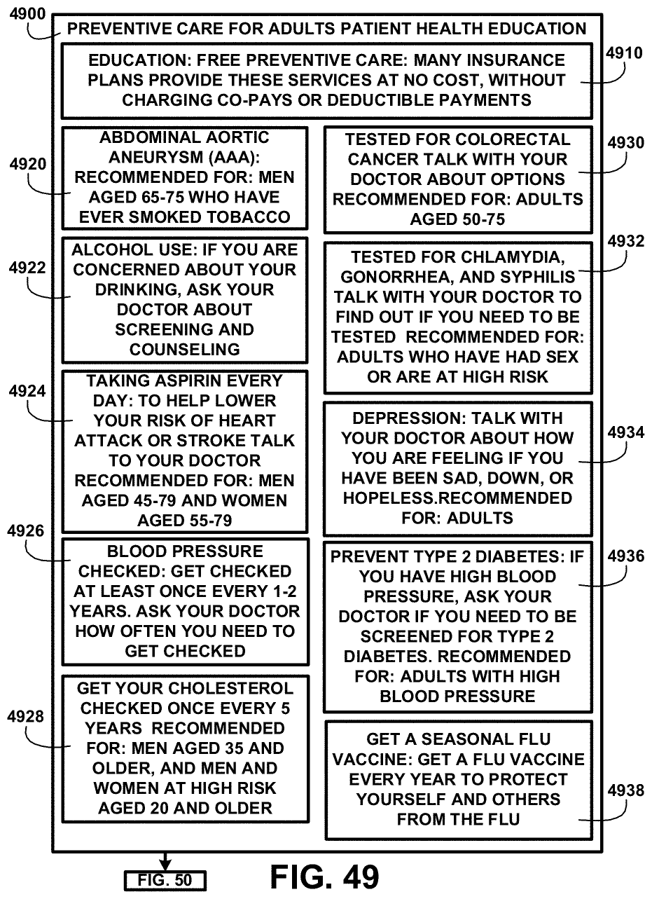

FIG. 49 shows for illustrative purposes only an example of preventive care for adults patient health education of one embodiment. FIG. 49 shows preventive care for adults patient health education 4900. Education: free preventive care: many insurance plans provide these services at no cost, without charging co-pays or deductible payments 4910. Abdominal aortic aneurysm (AAA): recommended for: men aged 65-75 who have ever smoked tobacco 4920. Alcohol use: if you are concerned about your drinking, ask your doctor about screening and counseling 4922.

Taking aspirin every day: to help lower your risk of heart attack or stroke talk to your doctor recommended for: men aged 45-79 and women aged 55-79 4924. Blood pressure checked: get checked at least once every 1-2 years. Ask your doctor how often you need to get checked 4926. Get your cholesterol checked once every 5 years recommended for: men aged 35 and older, and men and women at high risk aged 20 and older 4928. Tested for colorectal cancer talk with your doctor about options recommended for: adults aged 50-75 4930. Tested for chlamydia, gonorrhea, and syphilis talk with your doctor to find out if you need to be tested recommended for: adults who have had sex or are at high risk 4932.

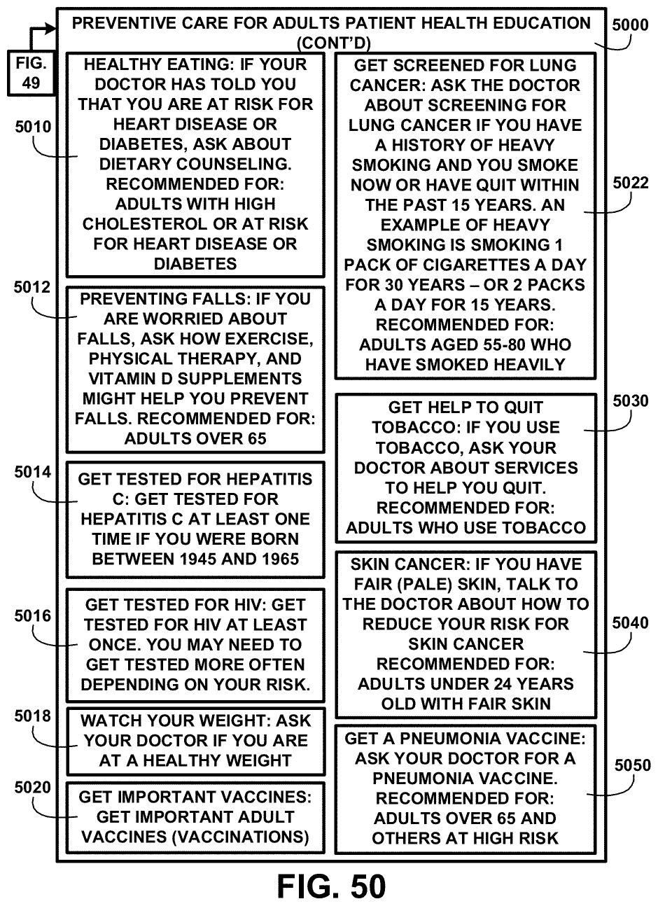

Depression: talk with your doctor about how you are feeling if you have been sad, down, or hopeless. Recommended for: adults 4934. Prevent type 2 diabetes: if you have high blood pressure, ask your doctor if you need to be screened for type 2 diabetes. Recommended for: adults with high blood pressure 4936. Get a seasonal flu vaccine: get a flu vaccine every year to protect yourself and others from the flu 4938 of one embodiment. Continued in FIG. 50.

### Preventive Care For Adults Patient Health Education (Cont'd):

FIG. 50 shows for illustrative purposes only an example of preventive care for adults patient health education (cont'd) of one embodiment. FIG. 50 shows preventive care for adults patient health education (cont'd) 5000 from FIG. 49. Healthy eating: if your doctor has told you that you are at risk for heart disease or diabetes, ask about dietary counseling. Recommended for: adults with high cholesterol or at risk for heart disease or diabetes 5010. Preventing falls: if you are worried about falls, ask how exercise, physical therapy, and vitamin d supplements might help you prevent falls. Recommended for: adults over 65 5012. Get tested for hepatitis C: get tested for hepatitis C at least one time if you were born between 1945 and 1965 5014.

Get tested for HIV: get tested for HIV at least once. You may need to get tested more often depending on your risk. 5016. Watch your weight: ask your doctor if you are at a healthy weight 5018. Get important vaccines: get important adult vaccines (vaccinations) 5020. Get screened for lung cancer: ask the doctor about screening for lung cancer if you have a history of heavy smoking and you smoke now or have quit within the past 15 years. An example of heavy smoking is smoking 1 pack of cigarettes a day for 30 years—or 2 packs a day for 15 years. Recommended for: adults aged 55-80 who have smoked heavily 5022. Get help to quit tobacco: if you use tobacco, ask your doctor about services to help you quit. Recommended for: adults who use tobacco 5030. Skin cancer: if you have fair (pale) skin, talk to the doctor about how to reduce your risk for skin cancer recommended for: adults under 24 years old with fair skin 5040. Get a pneumonia vaccine: ask your doctor for a pneumonia vaccine. Recommended for: adults over 65 and others at high risk 5050 of one embodiment.

### Preventive Care for Women Patient Health Education:

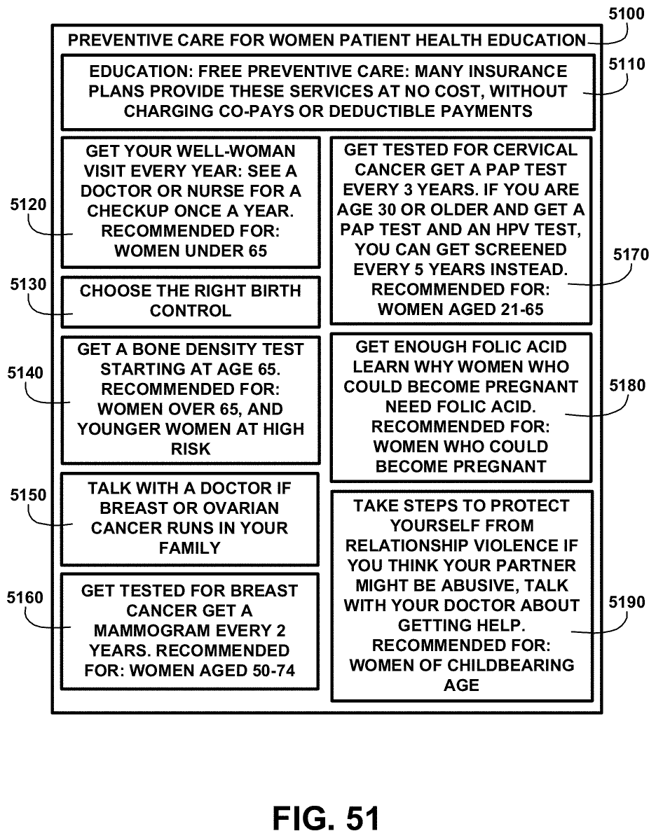

FIG. 51 shows for illustrative purposes only an example of preventive care for women patient health education of one embodiment. FIG. 51 shows preventive care for women patient health education 5100. Education: free preventive care: many insurance plans provide these services at no cost, without charging co-pays or deductible payments 5110. Get your well-woman visit every year: see a doctor or nurse for a checkup once a year. Recommended for: women under 65 5120. Choose the right birth control 5130. Get a bone density test, starting at age 65. Recommended for: women over 65, and younger women at high risk 5140. Talk with a doctor if breast or ovarian cancer runs in your family 5150. Get tested for breast cancer get a mammogram every 2 years. Recommended for: women aged 50-74 5160. Get tested for cervical cancer get a pap test every 3 years. If you are age 30 or older and get a pap test and an hpv test, you can get screened every 5 years instead. Recommended for: women aged 21-65 5170. Get enough folic acid learn why women who could become pregnant need folic acid. Recommended for: women who could become pregnant 5180. Take steps to protect yourself from relationship violence if you think your partner might be abusive, talk with your doctor about getting help. Recommended for: women of childbearing age 5190 of one embodiment.

### Preventive Care for Children Patient Health Education:

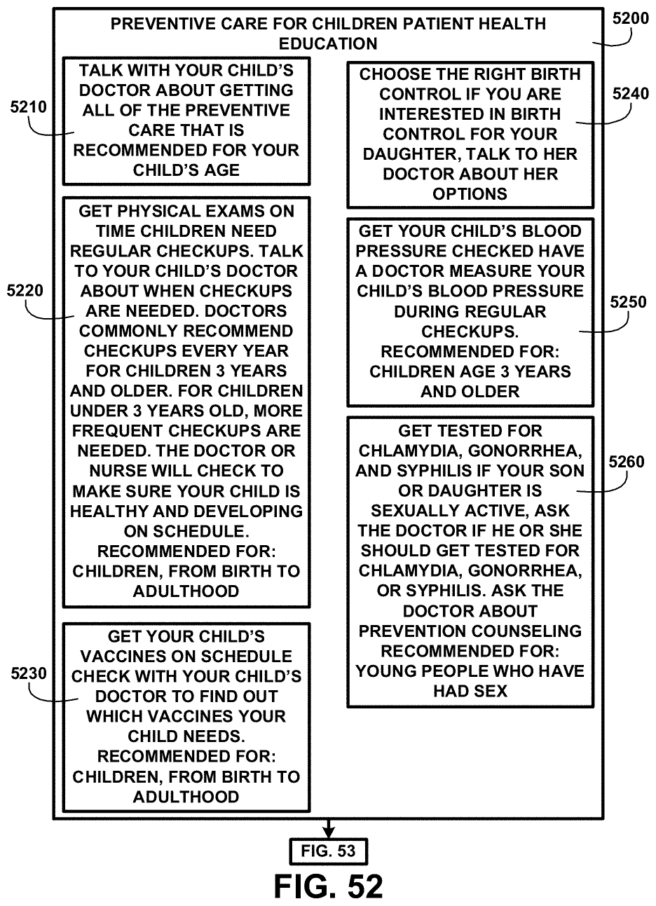

FIG. 52 shows for illustrative purposes only an example of preventive care for children patient health education of one embodiment. FIG. 52 shows preventive care for children patient health education 5200. The preventive care for children patient health education is important whether the child is the parent's first or fifteenth. Each child is different and can experience different health issues than their brothers and sisters. The ease with which the system makes this preventative education available to parents, grandparents and other relative close to the child makes this accessible and practical in our busy lives to avail ourselves of and detect and prevent a child from suffering complications from unattended conditions. In many cases early detection and treatment can relieve or cure the condition before a child additionally suffers mental and emotional problems because they are suffering alone.

Child preventative education begins with talk with your child's doctor about getting all of the preventive care that is recommended for your child's age 5210. Get physical exams on time children need regular checkups. Talk to your child's doctor about when checkups are needed. Doctors commonly recommend checkups every year for children 3 years and older. For children under 3 years old, more frequent checkups are needed. The doctor or nurse will check to make sure your child is healthy and developing on schedule. Recommended for: children, from birth to adulthood 5220. Get your child's vaccines on schedule check with your child's doctor to find out which vaccines your child needs. Recommended for: children, from birth to adulthood 5230.

Choose the right birth control if you are interested in birth control for your daughter, talk to her doctor about her options 5240. The system provides additional resources to assist parents in opening the discussions with their daughter on a subject that many parents find awkward and embarrassing. On all preventive care health education subject the system provides additional in depth materials to further explain a particular subject. In this subject of birth control information about teen pregnancies, abortion, and adoption as results of not exploring the preventative approach by using various birth control methods and medications. The more education on the subject the parent and child can review the better informed decisions they can make with their doctor's assistance.

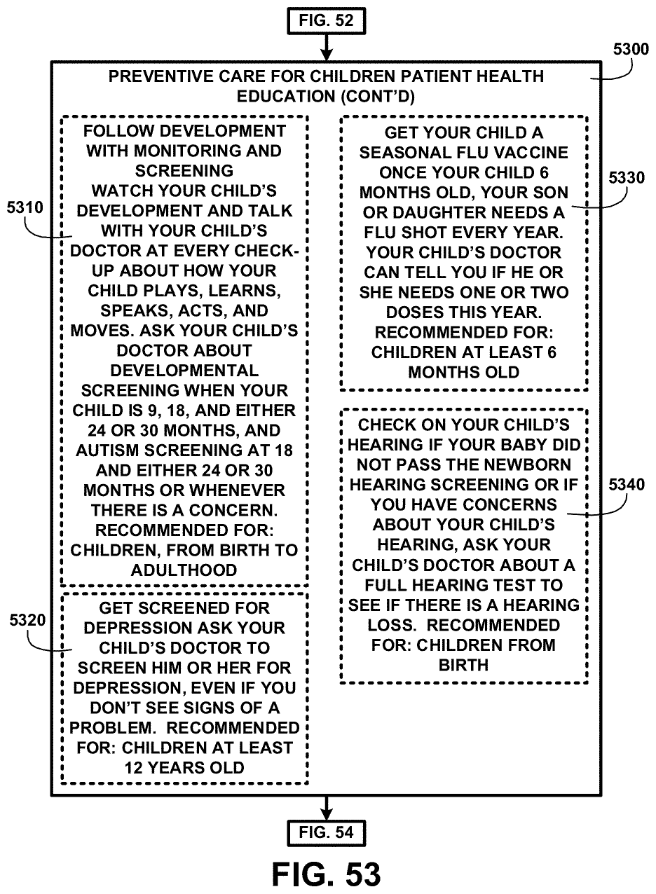

Get your child's blood pressure checked have a doctor measure your child's blood pressure during regular checkups. Recommended for: children age 3 years and older 5250. Get tested for chlamydia, gonorrhea, and syphilis if your son or daughter is sexually active, ask the doctor if he or she should get tested for chlamydia, gonorrhea, or syphilis. Ask the doctor about prevention counseling recommended for: young people who have had sex 5260 of one embodiment. The preventive care for children patient health education is continued in FIG. 53.

### Preventive Care for Children Patient Health Education (Cont'd):

FIG. 53 shows for illustrative purposes only an example of preventive care for children patient health education (cont'd) of one embodiment. FIG. 53 shows a continuation from FIG. 52 including preventive care for children patient health education (cont'd) 5300. Follow development with monitoring and screening watch your child's development and talk with your child's doctor at every check-up about how your child plays, learns, speaks, acts, and moves. Ask your child's doctor about developmental screening when your child is 9, 18, and either 24 or 30 months, and autism screening at 18 and either 24 or 30 months or whenever there is a concern. Recommended for: children, from birth to adulthood 5310. Get screened for depression ask your child's doctor to screen him or her for depression, even if you don't see signs of a problem. Recommended for: children at least 12 years old 5320. Get your child a seasonal flu vaccine once your child 6 months old, your son or daughter needs a flu shot every year. Your child's doctor can tell you if he or she needs one or two doses this year. Recommended for: children at least 6 months old 5330. Check on your child's hearing if your baby did not pass the newborn hearing screening or if you have concerns about your child's hearing, ask your child's doctor about a full hearing test to see if there is a hearing loss. Recommended for: children from birth 5340. A number of theses suggestion may not occur to a parent. We all think are children are perfect. A child with for example a hearing impairment does not know they are having difficult per se as they do not have reference point upon which to draw that conclusion. Creating benchmark testing will allow both the doctor and parents to better assess any current or future changes in a child's behavior that in this example would demonstrate that the child's hearing ability has changed and early detection can help the child in overcoming and dealing with the condition of one embodiment.

### Preventive Care for Children Patient Health Education (Cont'd):

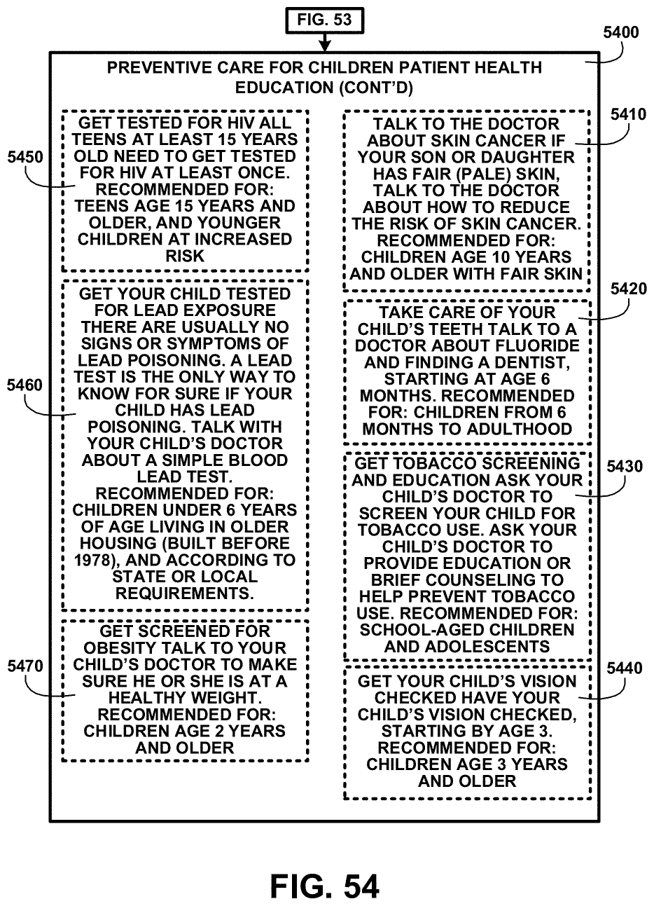

FIG. 54 shows for illustrative purposes only an example of preventive care for children patient health education (cont'd) of one embodiment. FIG. 54 shows a continuation from FIG. 53 preventive care for children patient health education (cont'd) 5400 including get tested for HIV all teens at least 15 years old need to get tested for HIV at least once. Recommended for: teens age 15 years and older, and younger children at increased risk 5410. Get your child tested for lead exposure there are usually no signs or symptoms of lead poisoning. A lead test is the only way to know for sure if your child has lead poisoning. Talk with your child's doctor about a simple blood lead test. Recommended for: children under 6 years of age living in older housing (built before 1978), and according to state or local requirements. 5420. Get screened for obesity talk to your child's doctor to make sure he or she is at a healthy weight. recommended for: children age 2 years and older 5430. Talk to the doctor about skin cancer if your son or daughter has fair (pale) skin, talk to the doctor about how to reduce the risk of skin cancer. Recommended for: children age 10 years and older with fair skin 5440. Take care of your child's teeth talk to a doctor about fluoride and finding a dentist, starting at age 6 months. Recommended for: children from 6 months to adulthood 5450. Get tobacco screening and education ask your child's doctor to screen your child for tobacco use. Ask your child's doctor to provide education or brief counseling to help prevent tobacco use. Recommended for: school-aged children and adolescents 5460 of one embodiment.

### Preventive Care for Pregnant Women Patient Health Education:

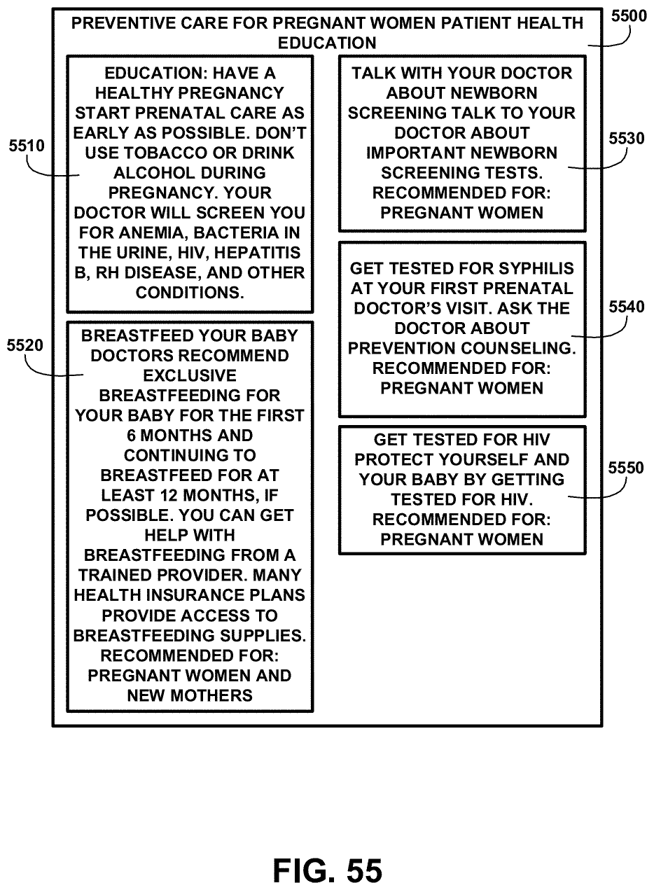

FIG. 55 shows for illustrative purposes only an example of preventive care for pregnant women patient health education of one embodiment. FIG. 55 shows preventive care for pregnant women patient health education 5500 including education: have a healthy pregnancy start prenatal care as early as possible. Don't use tobacco or drink alcohol during pregnancy. Your doctor will screen you for anemia, bacteria in the urine, HIV, hepatitis B, RH disease, and other conditions. 5510. Education for pregnant women is important where some women may not support of experienced mother's, and/or this is their first pregnancy. This system provides them with daily education to fit the daily nature of their pregnancy and where it would be difficult and expensive to contact their doctor's office daily with questions or to report what may be a normal pregnancy experience but for them it is a first-time experience. The examples of education available is to breastfeed your baby, doctors recommend exclusive breastfeeding for your baby for the first 6 months and continuing to breastfeed for at least 12 months, if possible. You can get help with breastfeeding from a trained provider. Many health insurance plans provide access to breastfeeding supplies. Recommended for: pregnant women and new mothers 5520. Talk with your doctor about newborn screening talk to your doctor about important newborn screening tests. Recommended for: pregnant women 5530. Get tested for syphilis at your first prenatal doctor's visit. Ask the doctor about prevention counseling. Recommended for: pregnant women 5540. Get tested for HIV protect yourself and your baby by getting tested for HIV. Recommended for: pregnant women 5550 of one embodiment.

### Patient Health Self-Assessments:

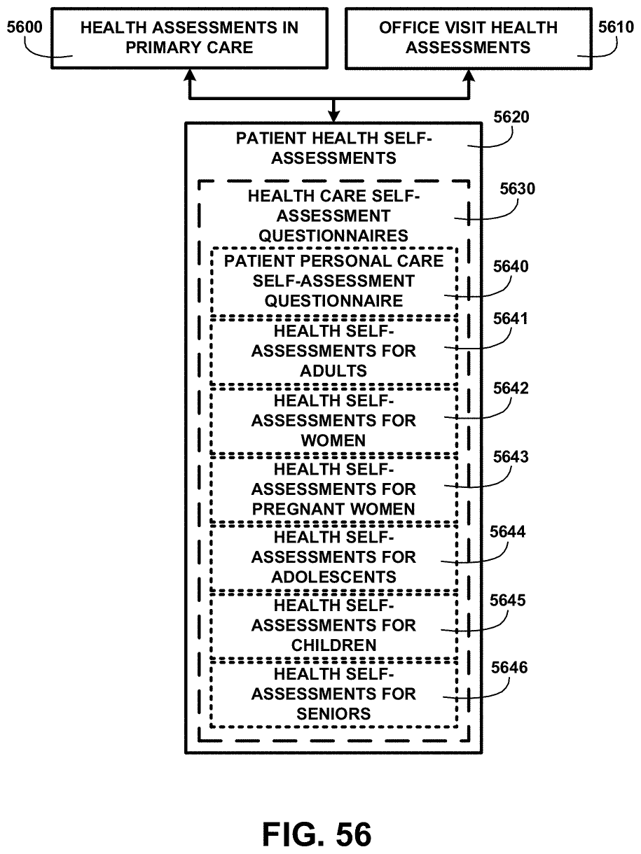

FIG. 56 shows for illustrative purposes only an example of patient health self-assessments of one embodiment. FIG. 56 shows a series of health assessments in primary care 5600, office visit health assessments 5610 and patient health self-assessments 5620. The patient self-assessment is a quality measure of the effectiveness of the preventative measures where the patient is communicating to their doctor on a continuing basis outside of office visits more frequently. Health care self-assessment questionnaires 5630 include questions and response to fit different genders, age groups and patient activities. Activities can be assessed using a patient personal care self-assessment questionnaire 5640 for assessing any difficulties a patient may be experiencing in perform daily living activities for example walking, preparing and eating meals. Other questionnaires include health self-assessments for adults 5641, health self-assessments for women 5642, health self-assessments for pregnant women 5643, health self-assessments for adolescents 5644, health self-assessments for children 5645, and health self-assessments for seniors 5646 of one embodiment.

### Activities of Daily Living (ADL) Self-Assessment:

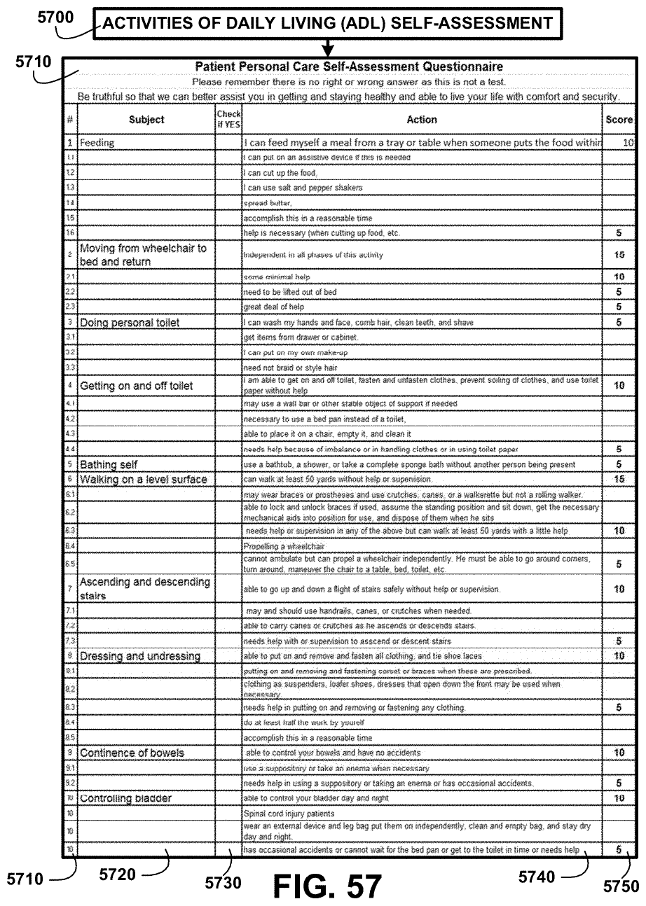

FIG. 57 shows for illustrative purposes only an example of activities of daily living (ADL) self-assessment of one embodiment. FIG. 57 shows an activities of daily living (ADL) self-assessment 5700 using a patient personal care self-assessment questionnaire 5710. The patient personal care self-assessment questionnaire 5710 records the behaviors and choices made by the patient during their daily actions. The artificial intelligence analyses these activities and may determine where the patient is making choices that will aggravate an existing condition. If that is determined the artificial intelligence will send an alert message to the patient informing the patient of why that choice is not helping management of their condition, may in fact create a new condition that is detrimental to their health and suggest alternative choices that will prevent those from occurring and still satisfy the patient's needs. This coaching and advice will assist the patient in preventing poor health choices and assist them in adjusting their life-style activities for healthier outcomes of one embodiment.

### Chronic Pain Assessment Questionnaire Part 1:

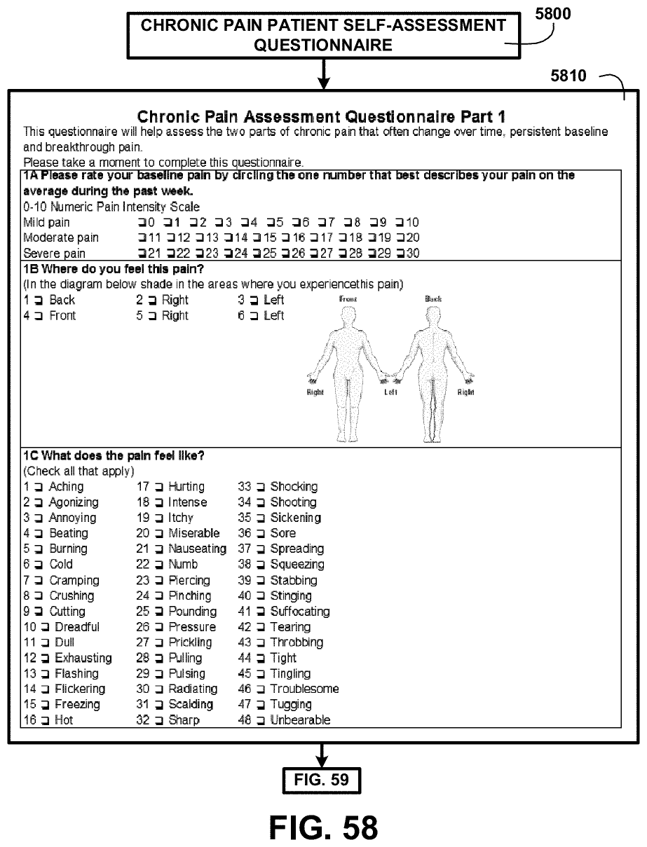

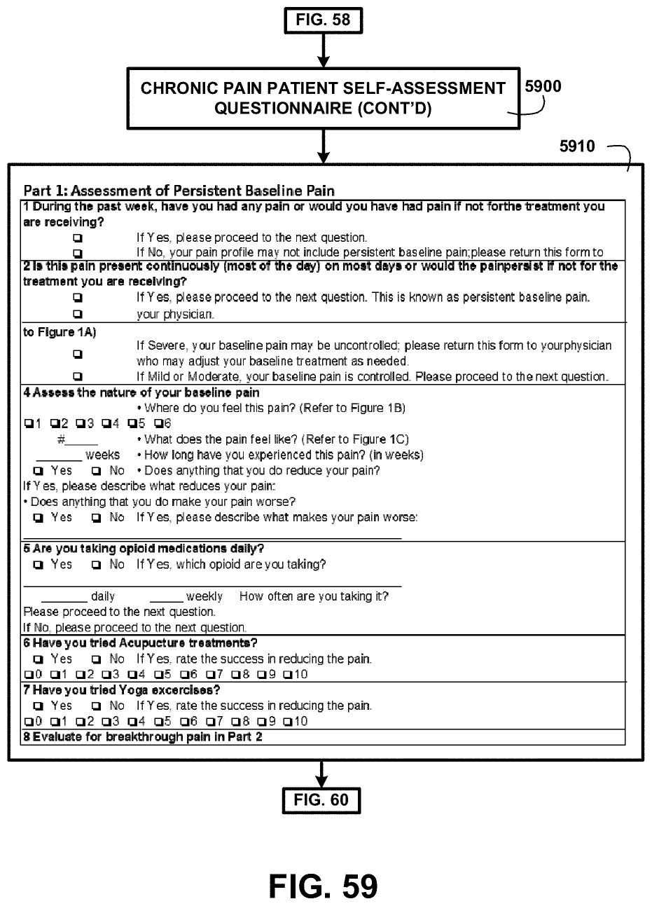

FIG. 58 shows for illustrative purposes only an example of chronic pain assessment questionnaire part 1 of one embodiment. FIG. 58 shows the first section of a chronic pain patient self-assessment questionnaire 5800. This chronic pain assessment questionnaire part 1 5810 allows the patient to provide information on the pain location and characteristics. The self-assessment form provides an easily filled out format to facilitate the patient's responses. The form is available electronically using a patient digital device including for example a smart phone, tablet or computer. The patient can push a tab on their digital device in response to an automatic reminder to access the self-assessment form and fill-in their responses, A patient may select a voice formatted operation where the questions and possible responses are spoken to the patient using the text to voice device 4620 of FIG. 46. The patient can speak their choice of response and the automated system will record the verbal response and additional convert it to a textual answer on the self-assessment form. The self-assessment form may also be presented to the patient in a language selected by the patient. The patient language selection is recorded in the patient's EHR. The text of the form is then translated into the selected language and the voice recognition 4630 of FIG. 46 device will translate the patient's response into English and fill-in the patient's response of one embodiment.

### Part 1: Assessment of Persistent Baseline Pain:

FIG. 59 shows for illustrative purposes only an example of part 1: assessment of persistent baseline pain of one embodiment. FIG. 59 shows chronic pain patient self-assessment questionnaire (cont'd) 5900 with part 1: assessment of persistent baseline pain 5910. These patient self-assessments will form a baseline of the conditions and activities that trigger the pain for the patient. The artificial intelligence analysis will weigh in the combination of patient ADLs and pain specific factors to develop alternative that for example may include medical device usage to relieve physical stresses of walking, reaching, bending over and other activities that initiate the pain. The analysis may also determine better pinpointing of the source of the pain between bone, muscles or soft tissues and alternative medications that can better delivery relief to those locations. Alternative well known treatments including for example acupuncture may be suggested where medications alone are not managing the patient's pain of one embodiment.

### Part 2: Assessment of Breakthrough Pain:

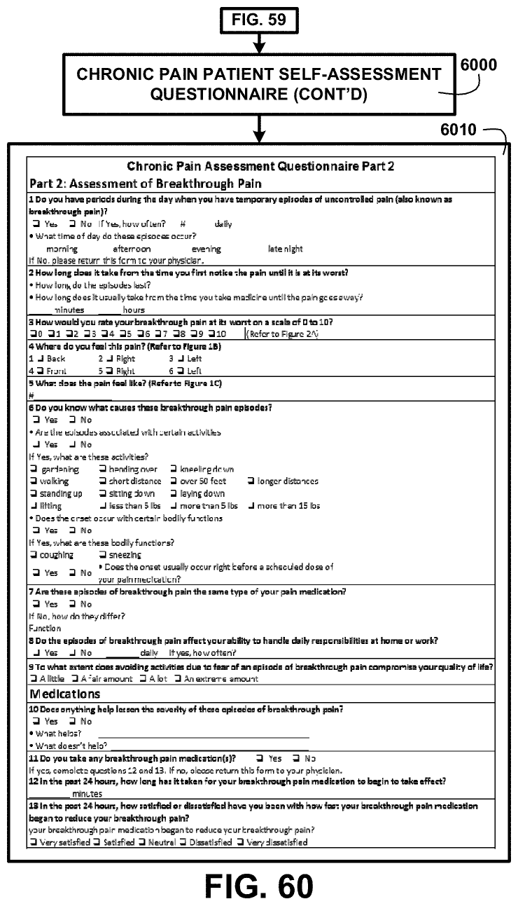

FIG. 60 shows for illustrative purposes only an example of part 2: assessment of breakthrough pain of one embodiment. FIG. 60 shows chronic pain patient self-assessment questionnaire (cont'd) 6000 with the chronic pain assessment questionnaire part 2 6010. The patient responses are analyzed to determine how well the patient is following the pain management plan provided by the doctor. The analysis also determines whether the patient may be becoming addicted to the medications for example an opioid. The analysis provides early detection of patient behaviors that can alert the doctor to potential addictive behaviors and alter medications to break the addictive chain before the patient develops a strong more difficult to treat addiction. The analysis also detects the mental and other factors that may be contributing to the patient turning to an abuse behavior to solve perceived problems other than just the pain. Counseling and alternative pain management may assist the patient to better deal with the pain and other issues that are prompting the abuse behaviors of one embodiment.

### Annual Wellness Visit:

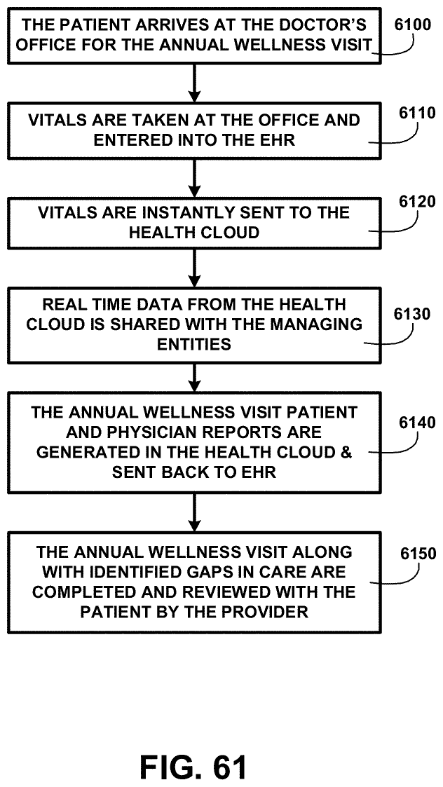

FIG. 61 shows a block diagram of an overview of annual wellness visit of one embodiment. FIG. 61 shows the patient arrives at the doctor's office for the annual wellness visit 6100. A portion of the quality measures assessments are performed including vitals are taken at the office and entered into the EHR 6110. Vitals are instantly sent to the health cloud 6120. Real time data from the health cloud is shared with the managing entities 6130 of the office and used in the artificial intelligence analyzing of the patients risk assessments. The annual wellness visit patient and physician reports are generated in the health cloud & sent back to EHR 6140. The annual wellness visit along with identified gaps in care are completed and reviewed with the patient by the provider 6150. These factors assist the provider and patient in developing and adjusting of the patient personalized prevention plan. The provider then confirms any adjustments in the patient personalized prevention plan. The data and reports generated by the artificial intelligence in HEDIS 4462 of FIG. 44 compliant reports of the quality measures and formatted into billing forms and the provider bills for the QPP 4464 of FIG. 44, MIPS 4466 of FIG. 44, and AAPM 4468 of FIG. 44 of one embodiment.

### Quality Measures Group 1:

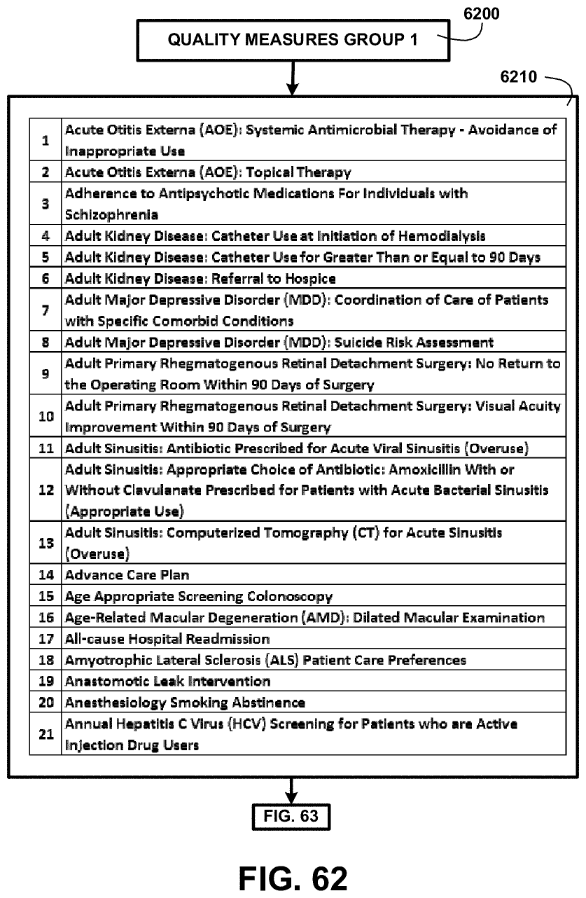

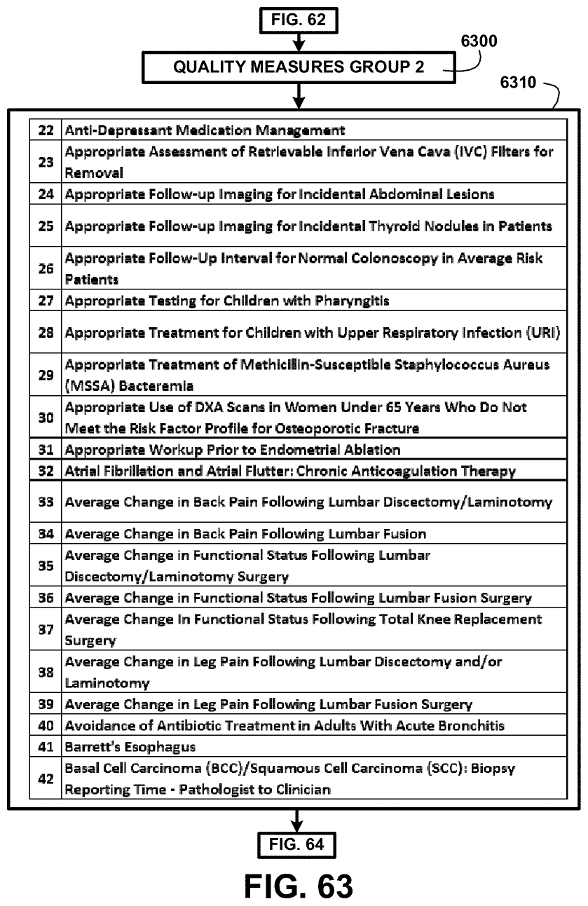

FIG. 62 shows a block diagram of an overview of quality measures group 1 of one embodiment. FIG. 62 shows quality measures group 1 6200 with a group 1 list 6210 including 1 Acute Otitis Externa (AOE): Systemic Antimicrobial Therapy—Avoidance of Inappropriate Use, 2 Acute Otitis Externa (AOE): Topical Therapy, 3 Adherence to Antipsychotic Medications For Individuals with Schizophrenia, 4 Adult Kidney Disease: Catheter Use at Initiation of Hemodialysis, 5 Adult Kidney Disease: Catheter Use for Greater Than or Equal to 90 Days, 6 Adult Kidney Disease: Referral to Hospice, 7 Adult Major Depressive Disorder (MDD): Coordination of Care of Patients with Specific Comorbid Conditions, 8 Adult Major Depressive Disorder (MDD): Suicide Risk Assessment, 9 Adult Primary Rhegmatogenous Retinal Detachment Surgery: No Return to the Operating Room Within 90 Days of Surgery, 10 Adult Primary Rhegmatogenous Retinal Detachment Surgery: Visual Acuity Improvement Within 90 Days of Surgery, 11 Adult Sinusitis: Antibiotic Prescribed for Acute Viral Sinusitis (Overuse), 12 Adult Sinusitis: Appropriate Choice of Antibiotic: Amoxicillin With or Without Clavulanate Prescribed for Patients with Acute Bacterial Sinusitis (Appropriate Use), 13 Adult Sinusitis: Computerized Tomography (CT) for Acute Sinusitis (Overuse), 14 Advance Care Plan, 15 Age Appropriate Screening Colonoscopy, 16 Age-Related Macular Degeneration (AMD): Dilated Macular Examination, 17 All-cause Hospital Readmission, 18 Amyotrophic Lateral Sclerosis (ALS) Patient Care Preferences, 19 Anastomotic Leak Intervention, 20 Anesthesiology Smoking Abstinence, and 21 Annual Hepatitis C Virus (HCV) Screening for Patients who are Active Injection Drug Users. The listing continues on FIG. 63.

### Quality Measures Group 2:

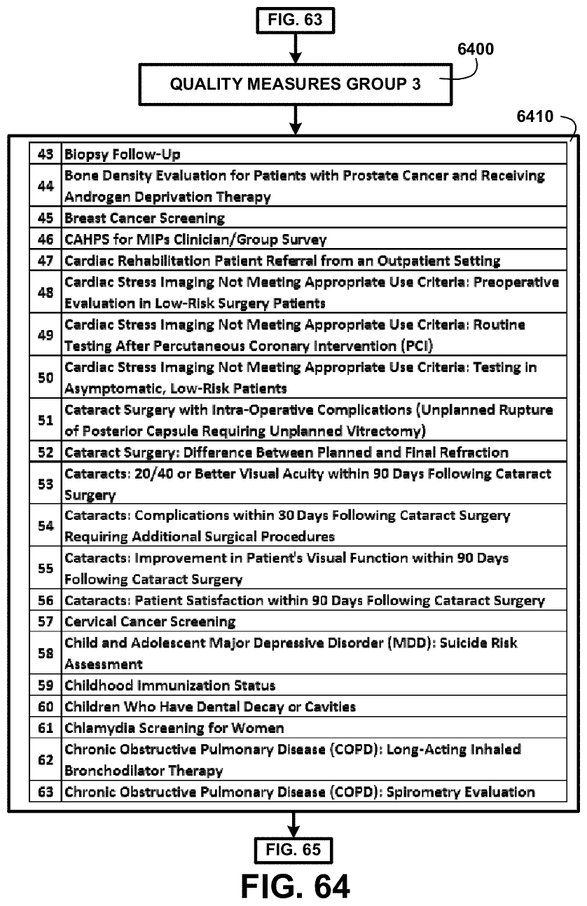

FIG. 63 shows a block diagram of an overview of quality measures group 2 of one embodiment. FIG. 63 shows continuing from FIG. 62 quality measures group 2 6300 including group 2 list 6310 with 22 Anti-Depressant Medication Management, 23 Appropriate Assessment of Retrievable Inferior Vena Cava (IVC) Filters for Removal, 24 Appropriate Follow-up Imaging for Incidental Abdominal Lesions, 25 Appropriate Follow-up Imaging for Incidental Thyroid Nodules in Patients, 26 Appropriate Follow-Up Interval for Normal Colonoscopy in Average Risk Patients, 27 Appropriate Testing for Children with Pharyngitis, 28 Appropriate Treatment for Children with Upper Respiratory Infection (URI), 29 Appropriate Treatment of Methicillin-Susceptible Staphylococcus Aureus (MSSA) Bacteremia, 30 Appropriate Use of DXA Scans in Women Under 65 Years Who Do Not Meet the Risk Factor Profile for Osteoporotic Fracture, 31 Appropriate Workup Prior to Endometrial Ablation, 32 Atrial Fibrillation and Atrial Flutter: Chronic Anticoagulation Therapy, 33 Average Change in Back Pain Following Lumbar Discectomy/Laminotomy, 34 Average Change in Back Pain Following Lumbar Fusion, 35 Average Change in Functional Status Following Lumbar Discectomy/Laminotomy Surgery, 36 Average Change in Functional Status Following Lumbar Fusion Surgery, 37 Average Change In Functional Status Following Total Knee Replacement Surgery, 38 Average Change in Leg Pain Following Lumbar Discectomy and/or Laminotomy, 39 Average Change in Leg Pain Following Lumbar Fusion Surgery, 40 Avoidance of Antibiotic Treatment in Adults With Acute Bronchitis, 41 Barrett's Esophagus, and 42 Basal Cell Carcinoma (BCC)/Squamous Cell Carcinoma (SCC): Biopsy Reporting Time—Pathologist to Clinician. The listing continues on FIG. 64.

### Quality Measures Group 3:

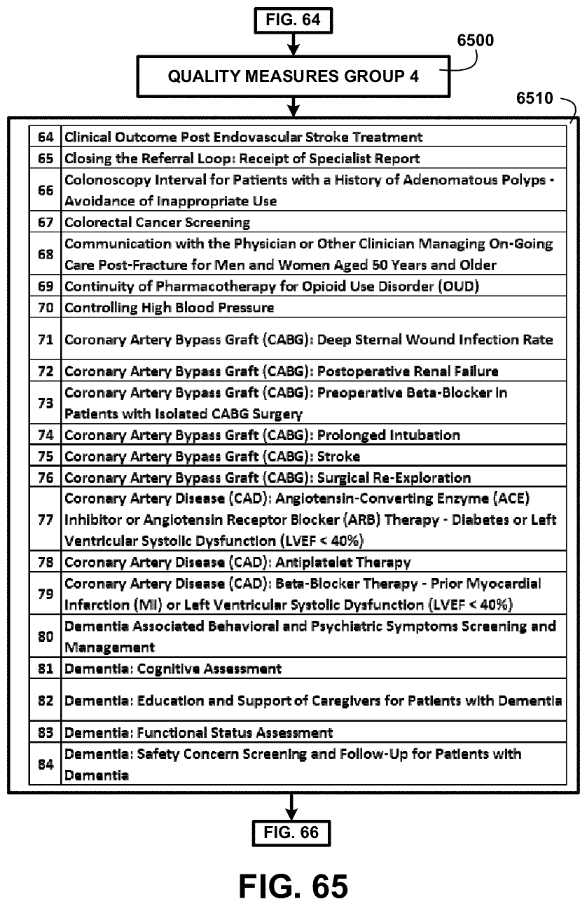

FIG. 64 shows a block diagram of an overview of quality measures group 3 of one embodiment. FIG. 64 shows continuing from FIG. 63 quality measures group 3 6400 including a group 3 list 6410 including 43 Biopsy Follow-Up, 44 Bone Density Evaluation for Patients with Prostate Cancer and Receiving Androgen Deprivation Therapy, 45 Breast Cancer Screening, 46 CAHPS for MIPs Clinician/Group Survey, 47 Cardiac Rehabilitation Patient Referral from an Outpatient Setting, 48 Cardiac Stress Imaging Not Meeting Appropriate Use Criteria: Preoperative Evaluation in Low-Risk Surgery Patients, 49 Cardiac Stress Imaging Not Meeting Appropriate Use Criteria: Routine Testing After Percutaneous Coronary Intervention (PCI), 50 Cardiac Stress Imaging Not Meeting Appropriate Use Criteria: Testing in Asymptomatic, Low-Risk Patients, 51 Cataract Surgery with Intra-Operative Complications (Unplanned Rupture of Posterior Capsule Requiring Unplanned Vitrectomy), 52 Cataract Surgery: Difference Between Planned and Final Refraction, 53 Cataracts: 20/40 or Better Visual Acuity within 90 Days Following Cataract Surgery, 54 Cataracts: Complications within 30 Days Following Cataract Surgery Requiring Additional Surgical Procedures, 55 Cataracts: Improvement in Patient's Visual Function within 90 Days Following Cataract Surgery, 56 Cataracts: Patient Satisfaction within 90 Days Following Cataract Surgery, 57 Cervical Cancer Screening, 58 Child and Adolescent Major Depressive Disorder (MDD): Suicide Risk Assessment, 59 Childhood Immunization Status, 60 Children Who Have Dental Decay or Cavities, 61 Chlamydia Screening for Women, 62 Chronic Obstructive Pulmonary Disease (COPD): Long-Acting Inhaled Bronchodilator Therapy, and 63 Chronic Obstructive Pulmonary Disease (COPD): Spirometry Evaluation. Listings continues on FIG. 65.

### Quality Measures Group 4:

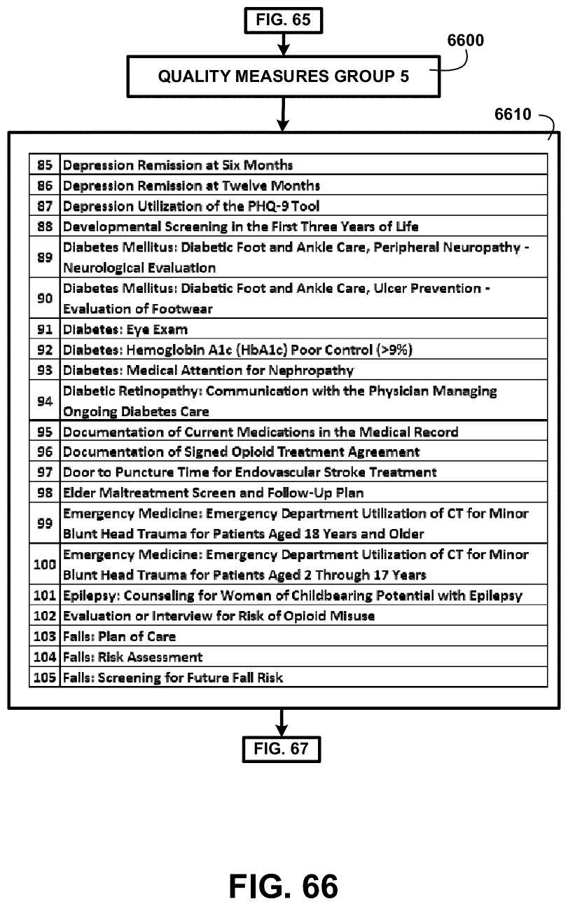

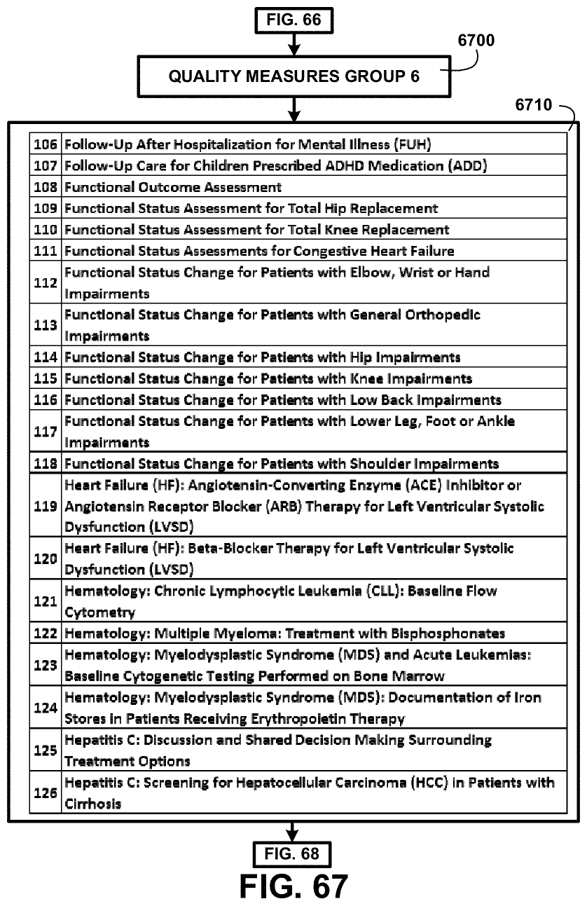

FIG. 65 shows for illustrative purposes only an example of quality measures group 4 of one embodiment. FIG. 65 shows continuing from FIG. 64 a quality measures group 4 6500 with a group 4 list 6510 including 64 Clinical Outcome Post Endovascular Stroke Treatment, 65 Closing the Referral Loop: Receipt of Specialist Report, 66 Colonoscopy Interval for Patients with a History of Adenomatous Polyps—Avoidance of Inappropriate Use, 67 Colorectal Cancer Screening, 68 Communication with the Physician or Other Clinician Managing On-Going Care Post-Fracture for Men and Women Aged 50 Years and Older, 69 Continuity of Pharmacotherapy for Opioid Use Disorder (OUD), 70 Controlling High Blood Pressure, 71 Coronary Artery Bypass Graft (CABG): Deep Sternal Wound Infection Rate, 72 Coronary Artery Bypass Graft (CABG): Postoperative Renal Failure, 73 Coronary Artery Bypass Graft (CABG): Preoperative Beta-Blocker in Patients with Isolated CABG Surgery, 74 Coronary Artery Bypass Graft (CABG): Prolonged Intubation, 75 Coronary Artery Bypass Graft (CABG): Stroke, 76 Coronary Artery Bypass Graft (CABG): Surgical Re-Exploration, 77 Coronary Artery Disease (CAD): Angiotensin-Converting Enzyme (ACE) Inhibitor or Angiotensin Receptor Blocker (ARB) Therapy—Diabetes or Left Ventricular Systolic Dysfunction (LVEF<40%), 78 Coronary Artery Disease (CAD): Antiplatelet Therapy, 79 Coronary Artery Disease (CAD): Beta-Blocker Therapy—Prior Myocardial Infarction (MI) or Left Ventricular Systolic Dysfunction (LVEF<40%), 80 Dementia Associated Behavioral and Psychiatric Symptoms Screening and Management, 81 Dementia: Cognitive Assessment, 82 Dementia: Education and Support of Caregivers for Patients with Dementia, 83 Dementia: Functional Status Assessment, and 84 Dementia: Safety Concern Screening and Follow-Up for Patients with Dementia. Listing of quality measures continues on FIG. 66.

### Quality Measures Group 5:

FIG. 66 shows for illustrative purposes only an example of quality measures group 5 of one embodiment. FIG. 66 shows a continuation from FIG. 65 of quality measures group 5 6600 with a group 5 list 6610 including 85 Depression Remission at Six Months, 86 Depression Remission at Twelve Months, 87 Depression Utilization of the PHQ-9 Tool, 88 Developmental Screening in the First Three Years of Life, 89 Diabetes Mellitus: Diabetic Foot and Ankle Care, Peripheral Neuropathy—Neurological Evaluation, 90 Diabetes Mellitus: Diabetic Foot and Ankle Care, Ulcer Prevention—Evaluation of Footwear, 91 Diabetes: Eye Exam, 92 Diabetes: Hemoglobin A1c (HbA1c) Poor Control (>9%), 93 Diabetes: Medical Attention for Nephropathy, 94 Diabetic Retinopathy: Communication with the Physician Managing Ongoing Diabetes Care, 95 Documentation of Current Medications in the Medical Record, 96 Documentation of Signed Opioid Treatment Agreement, 97 Door to Puncture Time for Endovascular Stroke Treatment, 98 Elder Maltreatment Screen and Follow-Up Plan, 99 Emergency Medicine: Emergency Department Utilization of CT for Minor Blunt Head Trauma for Patients Aged 18 Years and Older, 100 Emergency Medicine: Emergency Department Utilization of CT for Minor Blunt Head Trauma for Patients Aged 2 Through 17 Years, 101 Epilepsy: Counseling for Women of Childbearing Potential with Epilepsy, 102 Evaluation or Interview for Risk of Opioid Misuse, 103 Falls: Plan of Care, 104 Falls: Risk Assessment, and 105 Falls: Screening for Future Fall Risk. The listing continues on FIG. 67.

### Quality Measures Group 6:

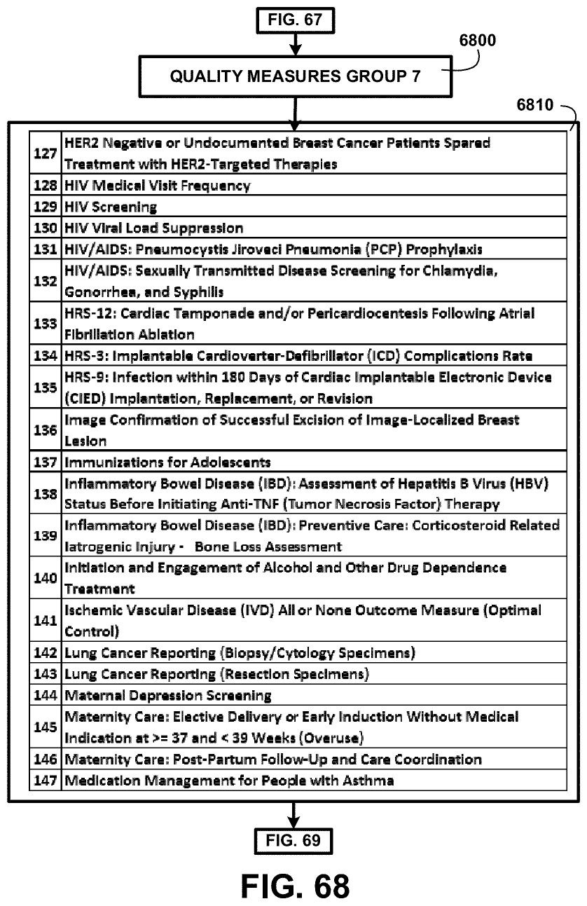

FIG. 67 shows for illustrative purposes only an example of quality measures group 6 of one embodiment. FIG. 67 shows a continuation from FIG. 66 showing quality measures group 6 6700 including a group 6 list 6710 including 106 Follow-Up After Hospitalization for Mental Illness (FUH), 107 Follow-Up Care for Children Prescribed ADHD Medication (ADD), 108 Functional Outcome Assessment, 109 Functional Status Assessment for Total Hip Replacement, 110 Functional Status Assessment for Total Knee Replacement, 111 Functional Status Assessments for Congestive Heart Failure, 112 Functional Status Change for Patients with Elbow, Wrist or Hand Impairments, 113 Functional Status Change for Patients with General Orthopedic Impairments, 114 Functional Status Change for Patients with Hip Impairments, 115 Functional Status Change for Patients with Knee Impairments, 116 Functional Status Change for Patients with Low Back Impairments, 117 Functional Status Change for Patients with Lower Leg, Foot or Ankle Impairments, 118 Functional Status Change for Patients with Shoulder Impairments, 119 Heart Failure (HF): Angiotensin-Converting Enzyme (ACE) Inhibitor or Angiotensin Receptor Blocker (ARB) Therapy for Left Ventricular Systolic Dysfunction (LVSD), 120 Heart Failure (HF): Beta-Blocker Therapy for Left Ventricular Systolic Dysfunction (LVSD), 121 Hematology: Chronic Lymphocytic Leukemia (CLL): Baseline Flow Cytometry, 122 Hematology: Multiple Myeloma: Treatment with Bisphosphonates, 123 Hematology: Myelodysplastic Syndrome (MDS) and Acute Leukemias: Baseline Cytogenetic Testing Performed on Bone Marrow, 124 Hematology: Myelodysplastic Syndrome (MDS): Documentation of Iron Stores in Patients Receiving Erythropoietin Therapy, 125 Hepatitis C: Discussion and Shared Decision Making Surrounding Treatment Options, and 126 Hepatitis C: Screening for Hepatocellular Carcinoma (HCC) in Patients with Cirrhosis. The listing of quality measures continues on FIG. 68.

### Quality Measures Group 7:

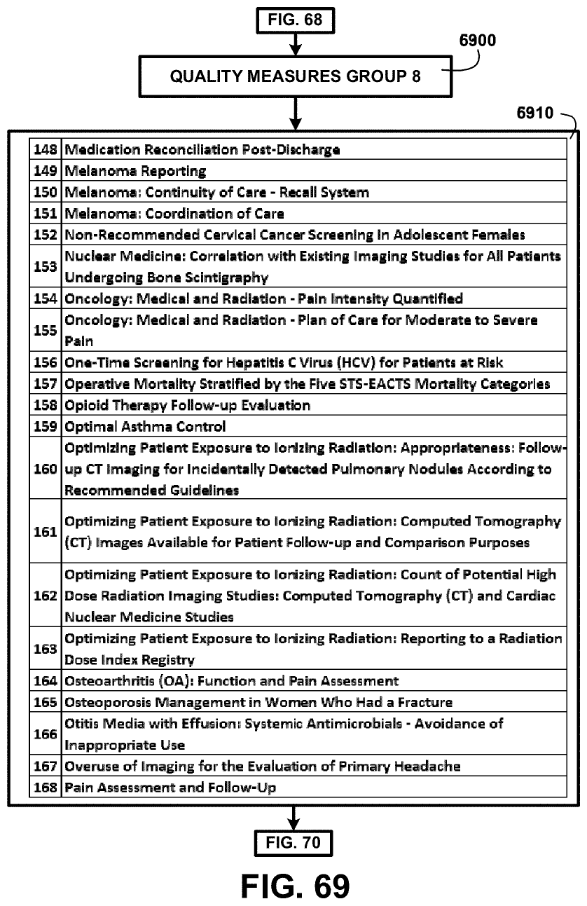

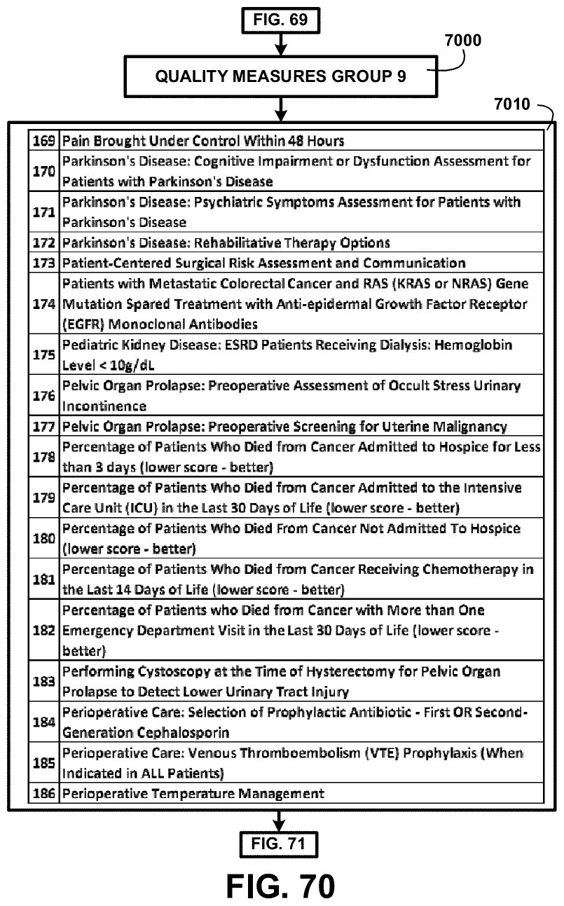

FIG. 68 shows for illustrative purposes only an example of quality measures group 7 of one embodiment. FIG. 68 shows a continuation from FIG. 67 including quality measures group 7 6800 with a group 7 list 6810 including 127 HER2 Negative or Undocumented Breast Cancer Patients Spared Treatment with HER2-Targeted Therapies, 128 HIV Medical Visit Frequency, 129 HIV Screening, 130 HIV Viral Load Suppression, 131 HIV/AIDS: Pneumocystis Jiroveci Pneumonia (PCP) Prophylaxis, 132 HIV/AIDS: Sexually Transmitted Disease Screening for Chlamydia, Gonorrhea, and Syphilis, 133 HRS-12: Cardiac Tamponade and/or Pericardiocentesis Following Atrial Fibrillation Ablation, 134 HRS-3: Implantable Cardioverter-Defibrillator (ICD) Complications Rate, 135 HRS-9: Infection within 180 Days of Cardiac Implantable Electronic Device (CIED) Implantation, Replacement, or Revision, 136 Image Confirmation of Successful Excision of Image-Localized Breast Lesion, 137 Immunizations for Adolescents, 138 Inflammatory Bowel Disease (IBD): Assessment of Hepatitis B Virus (HBV) Status Before Initiating Anti-TNF (Tumor Necrosis Factor) Therapy, 139 Inflammatory Bowel Disease (IBD): Preventive Care: Corticosteroid Related Iatrogenic Injury—Bone Loss Assessment, 140 Initiation and Engagement of Alcohol and Other Drug Dependence Treatment, 141 Ischemic Vascular Disease (IVD) All or None Outcome Measure (Optimal Control), 142 Lung Cancer Reporting (Biopsy/Cytology Specimens), 143 Lung Cancer Reporting (Resection Specimens), 144 Maternal Depression Screening, 145 Maternity Care: Elective Delivery or Early Induction Without Medical Indication at >=37 and <39 Weeks (Overuse), 146 Maternity Care: Post-Partum Follow-Up and Care Coordination, and 147 Medication Management for People with Asthma. The listing continue on FIG. 69.

### Quality Measures Group 8:

FIG. 69 shows for illustrative purposes only an example of quality measures group 8 of one embodiment. FIG. 69 shows a continuation from FIG. 68 including quality measures group 8 6900 with a group 8 list 6910 including 148 Medication Reconciliation Post-Discharge, 149 Melanoma Reporting, 150 Melanoma: Continuity of Care—Recall System, 151 Melanoma: Coordination of Care, 152 Non-Recommended Cervical Cancer Screening in Adolescent Females, 153 Nuclear Medicine: Correlation with Existing Imaging Studies for All Patients Undergoing Bone Scintigraphy, 154 Oncology: Medical and Radiation—Pain Intensity Quantified, 155 Oncology: Medical and Radiation—Plan of Care for Moderate to Severe Pain, 156 One-Time Screening for Hepatitis C Virus (HCV) for Patients at Risk, 157 Operative Mortality Stratified by the Five STS-EACTS Mortality Categories, 158 Opioid Therapy Follow-up Evaluation, 159 Optimal Asthma Control, 160 Optimizing Patient Exposure to Ionizing Radiation: Appropriateness: Follow-up CT Imaging for Incidentally Detected Pulmonary Nodules According to Recommended Guidelines, 161 Optimizing Patient Exposure to Ionizing Radiation: Computed Tomography (CT) Images Available for Patient Follow-up and Comparison Purposes, 162 Optimizing Patient Exposure to Ionizing Radiation: Count of Potential High Dose Radiation Imaging Studies: Computed Tomography (CT) and Cardiac Nuclear Medicine Studies, 163 Optimizing Patient Exposure to Ionizing Radiation: Reporting to a Radiation Dose Index Registry, 164 Osteoarthritis (OA): Function and Pain Assessment, 165 Osteoporosis Management in Women Who Had a Fracture, 166 Otitis Media with Effusion: Systemic Antimicrobials—Avoidance of Inappropriate Use, 167 Overuse of Imaging for the Evaluation of Primary Headache, and 168 Pain Assessment and Follow-Up. The next group is shown in FIG. 70.

### Quality Measures Group 9:

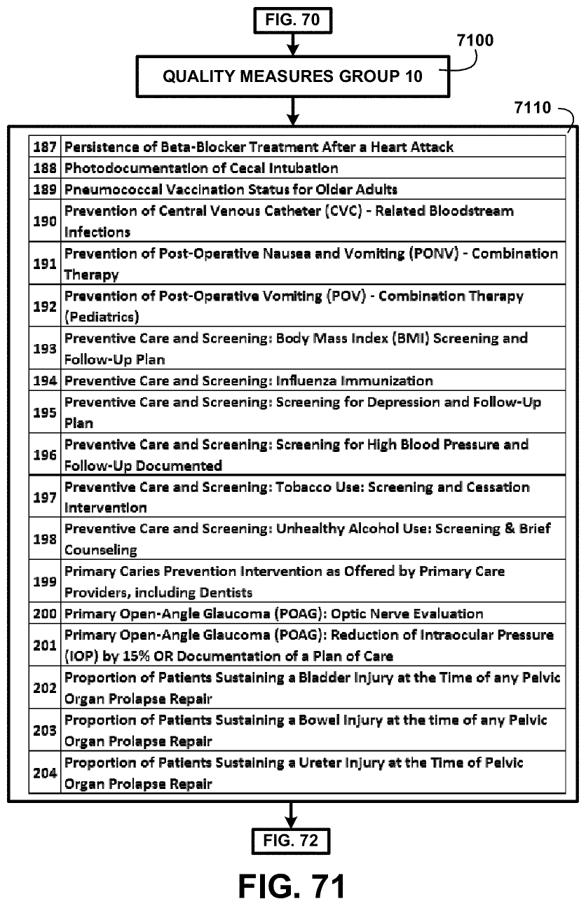

FIG. 70 shows for illustrative purposes only an example of quality measures group 9 of one embodiment. FIG. 70 shows continuing from FIG. 69 a quality measures group 9 7000 including group 9 list 7010 including 169 Pain Brought Under Control Within 48 Hours, 170 Parkinson's Disease: Cognitive Impairment or Dysfunction Assessment for Patients with Parkinson's Disease, 171 Parkinson's Disease: Psychiatric Symptoms Assessment for Patients with Parkinson's Disease, 172 Parkinson's Disease: Rehabilitative Therapy Options, 173 Patient-Centered Surgical Risk Assessment and Communication, 174 Patients with Metastatic Colorectal Cancer and RAS (KRAS or NRAS) Gene Mutation Spared Treatment with Anti-epidermal Growth Factor Receptor (EGFR) Monoclonal Antibodies, 175 Pediatric Kidney Disease: ESRD Patients Receiving Dialysis: Hemoglobin Level<10 g/dL, 176 Pelvic Organ Prolapse: Preoperative Assessment of Occult Stress Urinary Incontinence, 177 Pelvic Organ Prolapse: Preoperative Screening for Uterine Malignancy, 178 Percentage of Patients Who Died from Cancer Admitted to Hospice for Less than 3 days (lower score—better), 179 Percentage of Patients Who Died from Cancer Admitted to the Intensive Care Unit (ICU) in the Last 30 Days of Life (lower score—better), 180 Percentage of Patients Who Died From Cancer Not Admitted To Hospice (lower score—better), 181 Percentage of Patients Who Died from Cancer Receiving Chemotherapy in the Last 14 Days of Life (lower score—better), 182 Percentage of Patients who Died from Cancer with More than One Emergency Department Visit in the Last 30 Days of Life (lower score—better), 183 Performing Cystoscopy at the Time of Hysterectomy for Pelvic Organ Prolapse to Detect Lower Urinary Tract Injury, 184 Perioperative Care: Selection of Prophylactic Antibiotic—First OR Second-Generation Cephalosporin, 185 Perioperative Care: Venous Thromboembolism (VTE) Prophylaxis (When Indicated in ALL Patients), and 186 Perioperative Temperature Management. The list continues on FIG. 71.

### Quality Measures Group 10:

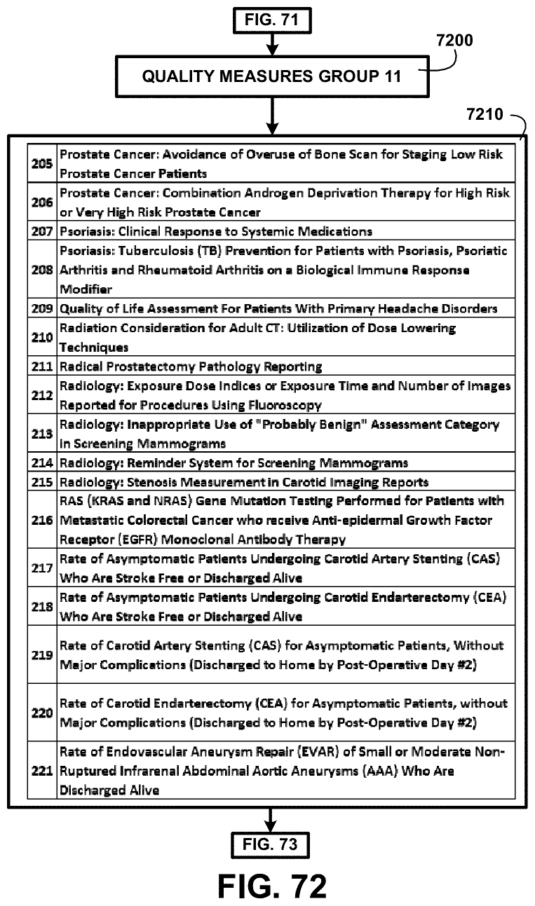

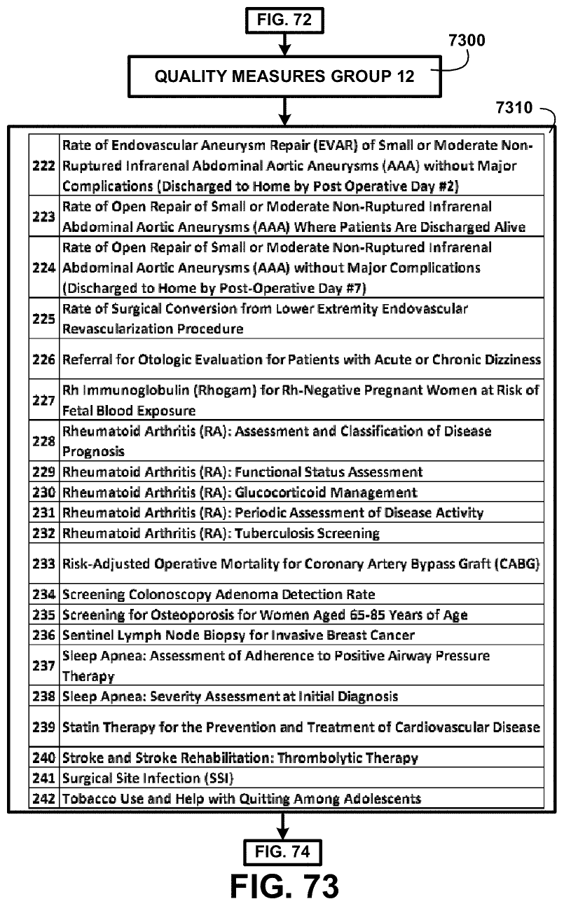

FIG. 71 shows for illustrative purposes only an example of quality measures group 10 of one embodiment. FIG. 71 shows a continuation from FIG. 70 showing quality measures group 10 7100 and a group 10 list 7110 including 187 Persistence of Beta-Blocker Treatment After a Heart Attack, 188 Photodocumentation of Cecal Intubation, 189 Pneumococcal Vaccination Status for Older Adults, 190 Prevention of Central Venous Catheter (CVC)-Related Bloodstream Infections, 191 Prevention of Post-Operative Nausea and Vomiting (PONV)—Combination Therapy, 192 Prevention of Post-Operative Vomiting (POV)-Combination Therapy (Pediatrics), 193 Preventive Care and Screening: Body Mass Index (BMI) Screening and Follow-Up Plan, 194 Preventive Care and Screening: Influenza Immunization, 195 Preventive Care and Screening: Screening for Depression and Follow-Up Plan, 196 Preventive Care and Screening: Screening for High Blood Pressure and Follow-Up Documented, 197 Preventive Care and Screening: Tobacco Use: Screening and Cessation Intervention, 198 Preventive Care and Screening: Unhealthy Alcohol Use: Screening & Brief Counseling, 199 Primary Caries Prevention Intervention as Offered by Primary Care Providers, including Dentists, 200 Primary Open-Angle Glaucoma (POAG): Optic Nerve Evaluation, 201 Primary Open-Angle Glaucoma (POAG): Reduction of Intraocular Pressure (IOP) by 15% OR Documentation of a Plan of Care, 202 Proportion of Patients Sustaining a Bladder Injury at the Time of any Pelvic Organ Prolapse Repair, 203 Proportion of Patients Sustaining a Bowel Injury at the time of any Pelvic Organ Prolapse Repair, and 204 Proportion of Patients Sustaining a Ureter Injury at the Time of Pelvic Organ Prolapse Repair. The list continues in FIG. 72.

### Quality Measures Group 11:

FIG. 72 shows for illustrative purposes only an example of quality measures group 11 of one embodiment. FIG. 72 shows a continuation from FIG. 71 showing quality measures group 11 7200. A group 11 list 7210 including 205 Prostate Cancer: Avoidance of Overuse of Bone Scan for Staging Low Risk Prostate Cancer Patients, 206 Prostate Cancer: Combination Androgen Deprivation Therapy for High Risk or Very High Risk Prostate Cancer, 207 Psoriasis: Clinical Response to Systemic Medications, 208 Psoriasis: Tuberculosis (TB) Prevention for Patients with Psoriasis, Psoriatic Arthritis and Rheumatoid Arthritis on a Biological Immune Response Modifier, 209 Quality of Life Assessment For Patients With Primary Headache Disorders, 210 Radiation Consideration for Adult CT: Utilization of Dose Lowering Techniques, 211 Radical Prostatectomy Pathology Reporting, 212 Radiology: Exposure Dose Indices or Exposure Time and Number of Images Reported for Procedures Using Fluoroscopy, 213 Radiology: Inappropriate Use of “Probably Benign” Assessment Category in Screening Mammograms, 214 Radiology: Reminder System for Screening Mammograms, 215 Radiology: Stenosis Measurement in Carotid Imaging Reports, 216 RAS (KRAS and NRAS) Gene Mutation Testing Performed for Patients with Metastatic Colorectal Cancer who receive Anti-epidermal Growth Factor Receptor (EGFR) Monoclonal Antibody Therapy, 217 Rate of Asymptomatic Patients Undergoing Carotid Artery Stenting (CAS) Who Are Stroke Free or Discharged Alive, 218 Rate of Asymptomatic Patients Undergoing Carotid Endarterectomy (CEA) Who Are Stroke Free or Discharged Alive, 219 Rate of Carotid Artery Stenting (CAS) for Asymptomatic Patients, Without Major Complications (Discharged to Home by Post-Operative Day #2), 220 Rate of Carotid Endarterectomy (CEA) for Asymptomatic Patients, without Major Complications (Discharged to Home by Post-Operative Day #2), and 221 Rate of Endovascular Aneurysm Repair (EVAR) of Small or Moderate Non-Ruptured Infrarenal Abdominal Aortic Aneurysms (AAA) Who Are Discharged Alive. Other quality measures continue in FIG. 73.

### Quality Measures Group 12:

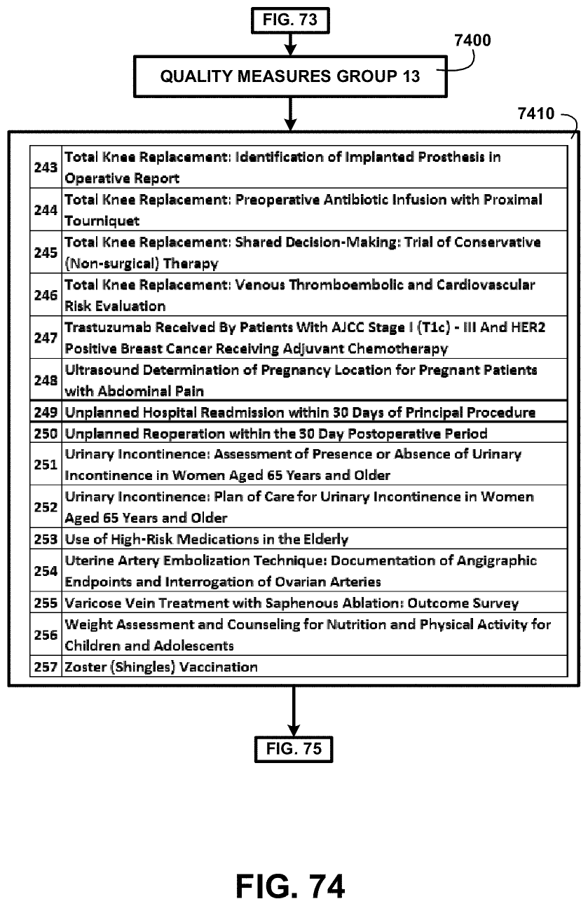

FIG. 73 shows for illustrative purposes only an example of quality measures group 12 of one embodiment. FIG. 73 shows continuing from FIG. 72 a quality measures group 12 7300 including a group 12 list 7310 including 222 Rate of Endovascular Aneurysm Repair (EVAR) of Small or Moderate Non-Ruptured Infrarenal Abdominal Aortic Aneurysms (AAA) without Major Complications (Discharged to Home by Post Operative Day #2), 223 Rate of Open Repair of Small or Moderate Non-Ruptured Infrarenal Abdominal Aortic Aneurysms (AAA) Where Patients Are Discharged Alive, 224 Rate of Open Repair of Small or Moderate Non-Ruptured Infrarenal Abdominal Aortic Aneurysms (AAA) without Major Complications (Discharged to Home by Post-Operative Day #7), 225 Rate of Surgical Conversion from Lower Extremity Endovascular Revascularization Procedure, 226 Referral for Otologic Evaluation for Patients with Acute or Chronic Dizziness, 227 Rh Immunoglobulin (Rhogam) for Rh-Negative Pregnant Women at Risk of Fetal Blood Exposure, 228 Rheumatoid Arthritis (RA): Assessment and Classification of Disease Prognosis, 229 Rheumatoid Arthritis (RA): Functional Status Assessment, 230 Rheumatoid Arthritis (RA): Glucocorticoid Management, 231 Rheumatoid Arthritis (RA): Periodic Assessment of Disease Activity, 232 Rheumatoid Arthritis (RA): Tuberculosis Screening, 233 Risk-Adjusted Operative Mortality for Coronary Artery Bypass Graft (CABG), 234 Screening Colonoscopy Adenoma Detection Rate, 235 Screening for Osteoporosis for Women Aged 65-85 Years of Age, 236 Sentinel Lymph Node Biopsy for Invasive Breast Cancer, 237 Sleep Apnea: Assessment of Adherence to Positive Airway Pressure Therapy, 238 Sleep Apnea: Severity Assessment at Initial Diagnosis, 239 Statin Therapy for the Prevention and Treatment of Cardiovascular Disease, 240 Stroke and Stroke Rehabilitation: Thrombolytic Therapy, 241 Surgical Site Infection (SSI), and 242 Tobacco Use and Help with Quitting Among Adolescents. The quality measures continue in FIG. 74.

### Quality Measures Group 13:

FIG. 74 shows for illustrative purposes only an example of quality measures group 13 of one embodiment. FIG. 74 shows a continuation from FIG. 73 with quality measures group 13 7400. FIG. 74 shows a group 13 list 7410 of quality measures for preventative health including 243 Total Knee Replacement: Identification of Implanted Prosthesis in Operative Report, 244 Total Knee Replacement: Preoperative Antibiotic Infusion with Proximal Tourniquet, 245 Total Knee Replacement: Shared Decision-Making: Trial of Conservative (Non-surgical) Therapy, 246 Total Knee Replacement: Venous Thromboembolic and Cardiovascular Risk Evaluation, 247 Trastuzumab Received By Patients With AJCC Stage I (T1c)-III And HER2 Positive Breast Cancer Receiving Adjuvant Chemotherapy, 248 Ultrasound Determination of Pregnancy Location for Pregnant Patients with Abdominal Pain, 249 Unplanned Hospital Readmission within 30 Days of Principal Procedure, 250 Unplanned Reoperation within the 30 Day Postoperative Period, 251 Urinary Incontinence: Assessment of Presence or Absence of Urinary Incontinence in Women Aged 65 Years and Older, 252 Urinary Incontinence: Plan of Care for Urinary Incontinence in Women Aged 65 Years and Older, 253 Use of High-Risk Medications in the Elderly, 254 Uterine Artery Embolization Technique: Documentation of Angigraphic Endpoints and Interrogation of Ovarian Arteries, 255 Varicose Vein Treatment with Saphenous Ablation: Outcome Survey, 256 Weight Assessment and Counseling for Nutrition and Physical Activity for Children and Adolescents, and 257 Zoster (Shingles) Vaccination of one embodiment.

### Benefits to Health Care Providers:

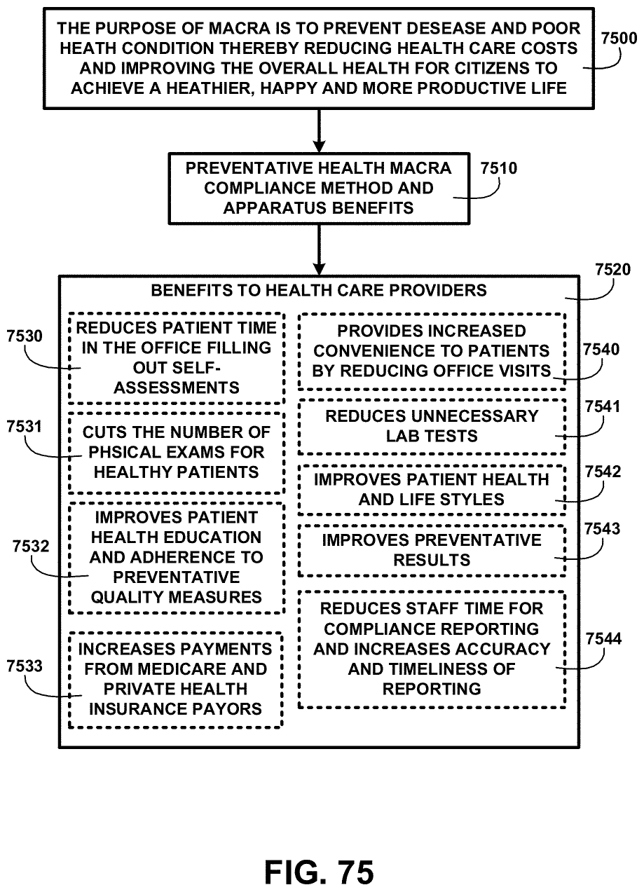

FIG. 75 shows for illustrative purposes only an example of benefits to health care providers of one embodiment. FIG. 75 shows the purpose of MACRA is to prevent disease and poor health condition thereby reducing health care costs and improving the overall health for citizens to achieve a heathier, happy and more productive life 7500. The integrated mobile device management system preventative health MACRA compliance method and apparatus benefits 7510 include benefits to health care providers 7520. The preventative health MACRA compliance method and apparatus reduces patient time in the office filling out self-assessments 7530, cuts the number of physical exams for healthy patients 7531, reduces unnecessary lab tests 7541, reduces staff time for compliance reporting and increases accuracy and timeliness of reporting 7544 and improves preventative results 7543.

Health care providers reduce cost due to the automated and convenient systems. The preventative health MACRA compliance method and apparatus improves patient health education and adherence to preventative quality measures 7532. The system provides increased convenience to patients by reducing office visits 7540 and providing the patient with easy access for Annual Wellness Visit self-assessment reporting. The preventative health MACRA compliance method and apparatus helps the treating doctor with remote monitoring of patient personalized prevention plan for tracking and analyzing patient progress. It also provides the patient with instant coaching and advice on keeping on track with their patient personalized prevention plan.

Doctors are able to spend less personal and staff time and still increase preventative care to their patients. MACRA provides the billing vehicles of the Quality Payment Program (QPP), Merit-based Incentive Payment System (MIPS) and Advanced Alternative Payment Model (AAPM) that increases payments from Medicare and private health insurance payors 7533. The preventative health MACRA compliance method and apparatus improves patient health education and adherence to preventative quality measures 7532 including the Healthcare Effectiveness Data and Information Set (HEDIS) set of performance measures. The integrated mobile device management system preventative health MACRA compliance method and apparatus benefits 7510 augments the health care providers preventative quality measures and reduces staff and office time to implement and maintain the preventative programs. This in turn provides greater emphasis for the patient to become more active in the preventative programs and quality measures that improves patient health and life styles 7542 of one embodiment.

The foregoing has described the principles, embodiments and modes of operation of the present invention. However, the invention should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

What is claimed is:

1. An integrated mobile device management method, comprising:
using an integrated mobile device management service provider digital programmable server and at least one database server for coordinating processing with device issuer locked devices with functionalities specifically targeted users to limit users access to specific functions;
coordinating locked devices functionalities for recording and analyzing user information, user device usage and sorting user profiles into layered categories;
analyzing data and controlling at least one specific function processes using at least one customized processor with an embedded algorithm within the integrated mobile device management service provider digital programmable server;
downloading locked device functions to at least one locked electronic device for device issuer distribution to targeted users;
using the integrated mobile device management service provider digital programmable server for operating an advertiser ad placement auction website;
selecting targeted advertisements based on user device usage analysis results; and
displaying targeted advertisements on at least one locked electronic devices.

2. The method of claim 1, wherein using the integrated mobile device management service provider digital programmable server includes using at least one programmable computer wherein the at least one programmable computer is configured to include digital processors, digital memory devices, digital communications devices, at least one WIFI device, GPS location devices, biometric scanners and analyzers, lie detection devices and functionality algorithm embedded processors.

3. The method of claim 1, wherein coordinating processing with device issuer locked devices includes coordinating processes including locked device functionalities downloaded into device issuer locked devices.

4. The method of claim 1, wherein distributing at least one locked electronic device to targeted users includes targeted users of layered user categories and layered sub-categories.

5. The method of claim 1, wherein selecting targeted advertisements based on user device usage analysis results includes user selected items of interest, user information, user profiles layered categories, user geographic location, and user on-line purchases.

6. The method of claim 1, wherein displaying targeted advertisements on at least one locked electronic devices includes using an integrated mobile device management service provider using device issuer authorized Internet service providers and search engines.

7. The method of claim 1, wherein using the integrated mobile device management service provider digital programmable server for operating an advertiser ad placement auction website includes displaying user layered categories and layered sub-categories usage percentages analysis results for selection of items of interest, device issuer authorized on-line purchases by geographic region, wherein advertisers bid on ad placement agreements.

8. The method of claim 1, wherein coordinating locked devices functionalities includes coordinating functions between the integrated mobile device management service provider digital programmable server and device issuer locked devices including limiting user access to specific functions, coordinating locked devices functionalities for recording and analyzing user information, user device usage and sorting user profiles into layered categories, analyzing data and controlling at least one specific function, wherein a specific function is configured to include a tracking targeted users physical locations, tracking social media websites usage, a doctor office appointment functionality, a background selfie images and audio lie detection function, an infectious disease screening function, an electronic mental health assessment function, hands-free locked device functionalities, a biometric devices user identification function, a designated locked schedule and locations function, a home care automated auditing system functionality, a universal automated patient intake system function and a multiple device issuer locked devices connectivity function.

9. The method of claim 1, wherein processing with device issuer locked devices includes processing with device issuer locked devices configured to include locked electronic devices including a computer, laptop, a portable electronic apparatus including a smart phone, electronic tablet, iPad, iPod, hands-free wearable health care devices including wearable wrist device, wearable ring device, Google glasses, wearable glasses device, wearable wrist band device, wearable pendant device and other digitally programmable devices.

10. The method of claim 1, wherein using an integrated mobile device management service provider digital programmable server for coordinating processing with device issuer locked devices with functionalities includes processing data captured, recorded and analyzed using device issuer locked devices features including at least one display screen, one or more led light, a thermographic camera, an ultraviolet/infrared camera, a video/still camera, at least one glasses lens and fiber optic cable, a retractable fiber optic light and flexible camera, a microphone, at least one speaker, infrared sensors, a heart beat detection sensor, micro-processors, touch screen operations areas, a temperature sensor, a GPS location device, a digital memory device, and at least one customized processor with an embedded algorithm.

11. An apparatus, comprising:
an integrated mobile device management service provider digital programmable server configured to include at least one database server, at least one programmable computer, at least one digital processor, at least one digital memory device, at least one digital communications device including at least one WIFI device, at least one GPS location device, at least one biometric scanner and biometric analyzer, at least one lie detection device, at least one customized processor with an embedded algorithm;
at least one device issuer locking functionality configured to include limiting user access to specific functions, coordinating locked devices functionalities for recording and analyzing user information, user device usage and sorting user profiles into layered categories, analyzing data and controlling at least one specific function, wherein a specific function is configured to include a tracking targeted users physical locations, tracking social media websites usage, a doctor office appointment functionality, a background selfie images and audio lie detection function, an infectious disease screening function, an electronic mental health assessment function, hands-free locked device functionalities, a biometric devices user identification function, a designated locked schedule and locations function, a home care automated auditing system functionality, a universal automated patient intake system function and a multiple device issuer locked devices connectivity function;
at least one integrated mobile device management service provider digital programmable server linked device issuer locked device configured to include locked electronic devices including a computer, laptop, a portable electronic apparatus including a smart phone, electronic tablet, iPad, iPod, hands-free wearable health care devices including wearable wrist device, wearable ring device, Google glasses, wearable glasses device, wearable wrist band device, wearable pendant device and other digitally programmable devices.

12. The apparatus of claim 11, wherein the integrated mobile device management service provider digital programmable server can be configured to develop ad placement locations for display on a locked device.

13. The apparatus of claim 11, wherein the integrated mobile device management service provider digital programmable server can be configured to develop locked device functionalities specifically targeted to device issuer users.

14. The apparatus of claim 11, wherein the integrated mobile device management service provider digital programmable server can be configured to provide home care visit DNA testing auditing using a locked device configured to include a home care visiting portable electronic apparatus.

15. The apparatus of claim 11, wherein the integrated mobile device management service provider digital programmable server can be configured to automatically update information in a database server for variety of databases.

16. An apparatus, comprising:
a preventative health MACRA compliance digital system for providing quality measures preventive care health services, screenings, patient health self-assessment, patient health education using a network server, plurality of databases, an artificial intelligence cloud and a network server computer;
a patient personalized prevention plan digital application configured for connection to an automated over the phone patient interface, and automated patient portal patient interface and a telephone call center, a patient's doctor's office, Medicare, and to the patient's health insurance provider;
an artificial intelligence cloud configured for analyzing quality measure data, preventative health results scoring and evaluations;
wherein the patient personalized prevention plan digital application is configured for remote monitoring of patient preventative quality measures progress; and
wherein the preventative health MACRA compliance digital system is configured for analyzing quality measures scoring, recording results in a patient HER, preparing compliance reports and assisting the doctor with evaluating the preventative measure results, preparing billing forms QPP, MIPS, and AAPM payments;

17. The apparatus of claim 16, wherein network server computer with the patient personalized prevention plan digital application is configured for sending alerts to a patient and a patient's doctor.

18. The apparatus of claim 16, wherein the patient personalized prevention plan digital application is configured for providing a patient with coaching and advice on keeping on track with the patient personalized prevention plan quality measures.

19. The apparatus of claim 16, wherein the preventative health MACRA compliance digital system is used for assisting medical health care providers with quality measures to prevent illnesses, disease, and other health problems, or to detect illness at an early stage when treatment is likely to work best.

20. The apparatus of claim 16, wherein the patient personalized prevention plan digital application is configured to provide monitoring patient food intact, exercise, vital signs, mental health condition, neurocognitive condition, monitors medication use, and monitors addictive medications use.
